SEQUENCE LISTING (94 sequences)

SEQ ID NO: 1 - protein, 203 aa, Artificial Sequence
  Synthetic polypeptide
ESGGRLVTPGGSLTLTCTVSGFSLSRYNMGWVRQAPGKGLEWIGWIPFRGSLKYATWATGRFTISRTSTTVDLRMTGLTAADTATYFCVRSSDGFDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 2 - protein, 207 aa, Artificial Sequence
  Synthetic polypeptide
ESGRGLVQPGGSLTLTCTASGFSIDSYGFSWVRQAPGKGLEHIGYLTAGGRAFYASWAKSRSTITRNTNENTVTLKMTSLTAADTATYFCAKLGSGNPVAIWGPGTLVTVSSGQPKAPSVFPLAPCCGDTPSSTVTLGCLVKGYLPEPVTVTWNSGTLTNGVRTFPAVRESSGLYSLNSVGKVTSSSQPVTCNVAHPATNTKVDKTV

SEQ ID NO: 3 - protein, 210 aa, Artificial Sequence
  Synthetic polypeptide
SVKESEGGLIKPGGILTLTCTASGFSLSSYGFSWVRQAPGKGLEHIGYLHANGRAYYATWAKSRSTITRNTNLNTVTLQLTSLTAADTATYFCAKIGSVSDVAIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 4 - protein, 210 aa, Artificial Sequence
  Synthetic polypeptide
SVKESEGGLFKPTDTLTLTCTVSGFSLTTYGFSWVRQAPGKGLEWIGYVRSDGRIYYTSWAKSRSTLTRNTNLNTVTLIMTSLTVADTATYFCAKIGSGTGVAIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 5 - protein, 216 aa, Artificial Sequence
  Synthetic polypeptide
AQFVMTQTPASVEAVVGGTVTIKCQASRDTGDGLIWYQQKPGQPPKRLIYKASTVASGVPSRFKGRGSGTDFTLTISDLECADAATYYCHSNFYNRWTYGNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 6 - protein, 217 aa, Artificial Sequence
  Synthetic polypeptide
DPVMTPTPSFTSAAVGGTVTINCQSSQSVWRNKNLAWYQQKPGQPPKRLIYAIATLDSGVPSRFSGSGSGTQFTLTISDVQCDDAATYYCVGHYGSENDAYYAFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 7 - protein, 223 aa, Artificial Sequence
  Synthetic polypeptide
DPVMTQTPSSTSAAVGGTVTINCQSSESIYNNKNLAWYQQKPGQSPRRLIYSISTLASGVSSRFKGSGSGTQFTLTISDVQCDDAATYYCVGYYYSGGYYYSGSAAYYAFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 8 - protein, 216 aa, Artificial Sequence
  Synthetic polypeptide
DGVMTPTPASASAGVGGTVTINCQSSQSIYKKYIAWYQQKPGQPPKRLIYSTSTLASGVSSRFKGSGSGTQFTLTISDVQCDDVATYYCVGYYIITNDAYYSFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 9 - protein, 10 aa, Artificial Sequence
  Synthetic peptide
CXXXXXXXXX

SEQ ID NO: 10 - protein, 21 aa, Artificial Sequence
  Synthetic peptide
DAVTYTEHAKRKTVTAMDVVY

SEQ ID NO: 11 - protein, 21 aa, Artificial Sequence
  Synthetic peptide
KYTKYSKHDMNKVLDLELKGD

SEQ ID NO: 12 - protein, 21 aa, Artificial Sequence
  Synthetic peptide
MKGVGTRHKALIRIMVSRSEI

SEQ ID NO: 13 - protein, 21 aa, Artificial Sequence
  Synthetic peptide
LKKALTGHLEEVVLALLKTPA

SEQ ID NO: 14 - protein, 19 aa, Artificial Sequence
  Synthetic peptide
GKGGAKRHRKVLRDNIQGI

SEQ ID NO: 15 - protein, 19 aa, Artificial Sequence
  Synthetic peptide
VITHGKGHYRIVKYSSVAN

SEQ ID NO: 16 - protein, 19 aa, Artificial Sequence
  Synthetic peptide
FRQVRLKHRKLREQVNSMV

SEQ ID NO: 17 - protein, 20 aa, Artificial Sequence
  Synthetic peptide
KRVLIAAHGNSLRGIVKHLE

SEQ ID NO: 18 - protein, 20 aa, Artificial Sequence
  Synthetic peptide
YKLVLIRHGESAWNLENRFS

SEQ ID NO: 19 - protein, 21 aa, Artificial Sequence
  Synthetic peptide
RCLFVCRHGERMDVVFGKYWL

SEQ ID NO: 20 - protein, 19 aa, Artificial Sequence
  Synthetic peptide
KSVLVVRHGERVDQIFGKA

SEQ ID NO: 21 - protein, 20 aa, Artificial Sequence
  Synthetic peptide
SSEVQFGHAGACANQASETA

SEQ ID NO: 22 - protein, 20 aa, Artificial Sequence
  Synthetic peptide
PPGRRMGHAGAIIAGGKGGA

SEQ ID NO: 23 - protein, 14 aa, Artificial Sequence
  Synthetic peptide
MKGVRMGHSGAIVE

SEQ ID NO: 24 - protein, 17 aa, Artificial Sequence
  Synthetic peptide
WRGQRDNAGAATEEFIK

SEQ ID NO: 25 - protein, 20 aa, Artificial Sequence
  Synthetic peptide
QELMTYSHDNIICGITSVSF

SEQ ID NO: 26 - protein, 20 aa, Artificial Sequence
  Synthetic peptide
GKCPLNPHSHLGTYGVFTNA

SEQ ID NO: 27 - protein, 20 aa, Artificial Sequence
  Synthetic peptide
CPLNPHSHLGTYGVFTNAAF

SEQ ID NO: 28 - protein, 20 aa, Artificial Sequence
  Synthetic peptide
NAIFRAAHSIKGGAGTFGFS

SEQ ID NO: 29 - protein, 20 aa, Artificial Sequence
  Synthetic peptide
HDIFRAAHTLKGMSATMGYT

SEQ ID NO: 30 - protein, 20 aa, Artificial Sequence
  Synthetic peptide
QVGRNIIHGSDSVKSAEKEI

SEQ ID NO: 31 - protein, 20 aa, Artificial Sequence
  Synthetic peptide
QVGRNIIHGSDSVESAEKEI

SEQ ID NO: 32 - protein, 20 aa, Artificial Sequence
  Synthetic peptide
VFIANISHELRTPLNGILGM

SEQ ID NO: 33 - protein, 20 aa, Artificial Sequence
  Synthetic peptide
TELDNLGHFLKGSSAALGLQ

SEQ ID NO: 34 - protein, 16 aa, Artificial Sequence
  Synthetic peptide
ANSRSSAHMSSNAIQR

SEQ ID NO: 35 - protein, 15 aa, Artificial Sequence
  Synthetic peptide
QVGRNIIHGSDSVES

SEQ ID NO: 36 - protein, 15 aa, Artificial Sequence
  Synthetic peptide
QVGRNIIHGSDSVKS

SEQ ID NO: 37 - protein, 15 aa, Artificial Sequence
  Synthetic peptide
HISRNVIHASDSVEG

SEQ ID NO: 38 - protein, 15 aa, Artificial Sequence
  Synthetic peptide
DDLRNALHGSNDFAA

SEQ ID NO: 39 - protein, 15 aa, Artificial Sequence
  Synthetic peptide
TDTRNTTHGSDSVVS

SEQ ID NO: 40 - protein, 15 aa, Artificial Sequence
  Synthetic peptide
DGIRNAAHGPDSFAS

SEQ ID NO: 41 - protein, 15 aa, Artificial Sequence
  Synthetic peptide
GKGGAKRHRKVLRDN

SEQ ID NO: 42 - protein, 15 aa, Artificial Sequence
  Synthetic peptide
FRQVRLKHRKLREQV

SEQ ID NO: 43 - protein, 15 aa, Artificial Sequence
  Synthetic peptide
LRQNTLGHLNLGLNL

SEQ ID NO: 44 - protein, 15 aa, Artificial Sequence
  Synthetic peptide
QELMTYSHDNIICGI

SEQ ID NO: 45 - protein, 15 aa, Artificial Sequence
  Synthetic peptide
YKLVLIRHGESAWNL

SEQ ID NO: 46 - protein, 15 aa, Artificial Sequence
  Synthetic peptide
SSEVQFGHAGACANQ

SEQ ID NO: 47 - protein, 15 aa, Artificial Sequence
  Synthetic peptide
PPGRRMGHAGAIIAG

SEQ ID NO: 48 - protein, 15 aa, Artificial Sequence
  Synthetic peptide
GKCPLNPHSHLGTYG

SEQ ID NO: 49 - protein, 15 aa, Artificial Sequence
  Synthetic peptide
GNNWAKGHYTEGAEL

SEQ ID NO: 50 - protein, 15 aa, Artificial Sequence
  Synthetic peptide
EETERSLHDAIMIVR

SEQ ID NO: 51 - protein, 15 aa, Artificial Sequence
  Synthetic peptide
KTALCFRHLMKQLLN

SEQ ID NO: 52 - protein, 15 aa, Artificial Sequence
  Synthetic peptide
GEMMDLQHGSLFLRT

SEQ ID NO: 53 - protein, 15 aa, Artificial Sequence
  Synthetic peptide
LGKLMELHGEGSSSG

SEQ ID NO: 54 - protein, 15 aa, Artificial Sequence
  Synthetic peptide
TMEKAGAHLQGGAKR

SEQ ID NO: 55 - protein, 15 aa, Artificial Sequence
  Synthetic peptide
CGERLYDLNMPAYVK

SEQ ID NO: 56 - protein, 15 aa, Artificial Sequence
  Synthetic peptide
CGERLYDLNMPAYVK

SEQ ID NO: 57 - protein, 10 aa, Artificial Sequence
  Synthetic peptide
CLKPLKTYVD

SEQ ID NO: 58 - protein, 10 aa, Artificial Sequence
  Synthetic peptide
CLKPLKTYVD

SEQ ID NO: 59 - protein, 21 aa, Artificial Sequence
  Synthetic peptide
AVSVSETDDYAEIIDEEDTYT

SEQ ID NO: 60 - DNA, 30 bp, Artificial Sequence
  Synthetic polynucleotide
gatcggatccatggccaactgtgagcgtac

SEQ ID NO: 61 - DNA, 31 bp, Artificial Sequence
  Synthetic polynucleotide
gatcgaattctcattcatagatccagttctc

SEQ ID NO: 62 - DNA, 30 bp, Artificial Sequence
  Synthetic polynucleotide
gatcggatccatggccaacctggagcgcac

SEQ ID NO: 63 - DNA, 31 bp, Artificial Sequence
  Synthetic polynucleotide
gatcgaattcttattcatagacccagtcatg

SEQ ID NO: 64 - DNA, 31 bp, Artificial Sequence
  Synthetic polynucleotide
gatcggatccatggccgcctacaaactggtg

SEQ ID NO: 65 - DNA, 31 bp, Artificial Sequence
  Synthetic polynucleotide
gatcgaattctcacttcttggccttgccctg

SEQ ID NO: 66 - DNA, 18 bp, Artificial Sequence
  Synthetic polynucleotide
gtgatgacccagactcca

SEQ ID NO: 67 - DNA, 18 bp, Artificial Sequence
  Synthetic polynucleotide
gtgctgacccagactcca

SEQ ID NO: 68 - DNA, 18 bp, Artificial Sequence
  Synthetic polynucleotide
gatatgacccagactcca

SEQ ID NO: 69 - DNA, 18 bp, Artificial Sequence
  Synthetic polynucleotide
gatctgacccagactcca

SEQ ID NO: 70 - DNA, 21 bp, Artificial Sequence
  Synthetic polynucleotide
tttgatttccacattggtgcc

SEQ ID NO: 71 - DNA, 21 bp, Artificial Sequence
  Synthetic polynucleotide
taggatctccagctcggtccc

SEQ ID NO: 72 - DNA, 21 bp, Artificial Sequence
  Synthetic polynucleotide
tttgaccaccacctcggtccc

SEQ ID NO: 73 - DNA, 21 bp, Artificial Sequence
  Synthetic polynucleotide
tttgacgaccacctcggtccc

SEQ ID NO: 74 - DNA, 20 bp, Artificial Sequence
  Synthetic polynucleotide
gtgctgactcagtcgccctc

SEQ ID NO: 75 - DNA, 24 bp, Artificial Sequence
  Synthetic polynucleotide
gcctgtgacggtcagctgggtccc

SEQ ID NO: 76 - DNA, 20 bp, Artificial Sequence
  Synthetic polynucleotide
agtcggtggaggagtccagg

SEQ ID NO: 77 - DNA, 20 bp, Artificial Sequence
  Synthetic polynucleotide
agtcggtggaggagtccggg

SEQ ID NO: 78 - DNA, 20 bp, Artificial Sequence
  Synthetic polynucleotide
agtcggtgaaggagtccgag

SEQ ID NO: 79 - DNA, 20 bp, Artificial Sequence
  Synthetic polynucleotide
agtcgctggaggagtccggg

SEQ ID NO: 80 - DNA, 20 bp, Artificial Sequence
  Synthetic polynucleotide
agtcgttggaggagtccggg

SEQ ID NO: 81 - DNA, 22 bp, Artificial Sequence
  Synthetic polynucleotide
agcagcagctgatggagtccgg

SEQ ID NO: 82 - DNA, 22 bp, Artificial Sequence
  Synthetic polynucleotide
aggagcagctgatggagtccgg

SEQ ID NO: 83 - DNA, 22 bp, Artificial Sequence
  Synthetic polynucleotide
agcagcagctggtggagtccgg

SEQ ID NO: 84 - DNA, 22 bp, Artificial Sequence
  Synthetic polynucleotide
aggagcagctggtggagtccgg

SEQ ID NO: 85 - DNA, 21 bp, Artificial Sequence
  Synthetic polynucleotide
agagacggtgaccagggtgcc

SEQ ID NO: 86 - DNA, 21 bp, Artificial Sequence
  Synthetic polynucleotide
ggagacggtgaccagggtgcc

SEQ ID NO: 87 - DNA, 21 bp, Artificial Sequence
  Synthetic polynucleotide
agagatggtgaccagggtgcc

SEQ ID NO: 88 - DNA, 21 bp, Artificial Sequence
  Synthetic polynucleotide
ggagatggtgaccagggtgcc

SEQ ID NO: 89 - protein, 9 aa, Artificial Sequence
  Synthetic peptide
AGAGXAGAG

SEQ ID NO: 90 - protein, 9 aa, Artificial Sequence
  Synthetic peptide
VLIRXGESA

SEQ ID NO: 91 - protein, 9 aa, Artificial Sequence
  Synthetic peptide
RNIIXGSDS

SEQ ID NO: 92 - protein, 9 aa, Artificial Sequence
  Synthetic peptide
GAKRXRKVL

SEQ ID NO: 93 - protein, 9 aa, Artificial Sequence
  Synthetic peptide
VRLKXRKLR

SEQ ID NO: 94 - protein, 9 aa, Artificial Sequence
  Synthetic peptide
MTYSXDNII

(19) United States
(12) Patent Application Publication
(10) Pub. No.: US 2016/0033516 A1
(43) Pub. Date: Feb. 4, 2016

(54) USE OF POLYCLONAL AND MONOCLONAL ANTIBODIES SPECIFIC FOR 3-PHOSPHOHISTIDINE

(71) Applicant: SALK INSTITUTE FOR BIOLOGICAL STUDIES, La Jolla, CA (US)

(72) Inventors: Tony Hunter, Del Mar, CA (US); Stephen Rush Fuhs, San Diego, CA (US); Jill Meisenhelder, Vista, CA (US)

(73) Assignee: SALK INSTITUTE FOR BIOLOGICAL STUDIES, La Jolla, CA (US)

(21) Appl. No.: 14/789,811

(22) Filed: Jul. 1, 2015

Related U.S. Application Data
(60) Provisional application No. 62/031,796, filed on Jul. 31, 2014.

(51) Int. Cl. G01N 33/574; G01N 33/68; G01N 33/573

(52) U.S. Cl. CPC G01N 33/57496; G01N 33/573; G01N 33/6854; G01N 2800/52

(57) ABSTRACT

Isolated monoclonal antibodies and antigen binding fragments are disclosed herein that specifically bind polypeptides comprising a histidine phosphorylated at N3 (3-pHis). Nucleic acids encoding these antibodies, vectors including these nucleic acids, and host cells transformed with these vectors and nucleic acids are also disclosed. Methods are also disclosed for using these antibodies, such as for detection of polypeptides comprising a histidine phosphorylated at N3 (3-pHis), detection of a tumor, monitoring the effectiveness of therapeutic agent, and identifying antibiotics. In some embodiments, the methods can be used to investigate signal transduction pathways.

### CROSS REFERENCE TO RELATED APPLICATIONS

This claims the benefit of U.S. Application No. 62/031,796, filed Jul. 31, 2014, which is incorporated by reference herein.

### ACKNOWLEDGMENT OF GOVERNMENT SUPPORT

This invention was made with government support under grant no. 5 RO 1 CA082683-15 awarded by the National Institutes of Health and grant no. 5 T32 CA009370-31 from the National Institutes of Health. The government has certain rights in the invention.

### FIELD

This relates to the field of antibodies, specifically to the use of antibodies that specifically bind a polypeptide that includes a histidine phosphorylated at N3 (3-pHis), such as for the identification of antibiotics and detecting the presence of a tumor in a subject.

### BACKGROUND

The majority of intracellular proteins are phosphorylated at any given time, and, while nine of the 20 amino acids can be phosphorylated, the current focus has been on serine (Ser), threonine (Thr), and tyrosine (Tyr) phosphorylation despite pHis having been first identified over 50 years ago (Boyer, J. Biol. Chem., 3306 (1962)). These OH-containing amino acids form acid-stable, phosphoester (P—O) bonds upon phosphorylation (Attwood, et al., Amino acids 32, 145 (January, 2007)). Histidine (His) forms a heat and acid-labile phosphoramidate (P—N) bond when phosphorylated. Phosphospecific antibodies have enabled the routine study of phosphoester protein phosphorylation, and the use of MS-proteomics has identified over 200,000 non-redundant sites of phosphorylation (Hornbeck et al., Nucl. acids res 40, D261 (January, 2012)). The lack of specific antibodies to study pHis and the relative instability of the P—N bond under typical conditions used for proteomics have made it impossible to determine the prevalence of pHis, although it has been estimated that up to 6% of phosphorylation in eukaryotes occurs on His (Matthews, Pharmac. Ther. 67, 232 (1995)). Thus, it is possible that phosphohistidine (pHis) could be more abundant than phosphotyrosine (pTyr), which, despite its importance, comprises ˜1% of all known phosphorylation sites (Hunter and Sefton, Proc. Natl. Acad. Sci. USA 77, 1311 (Mar. 1, 1980, 1980); Olsen et al., Cell 127, 635 (Nov. 3, 2006)). Since current biochemical and proteomic technologies have been optimized for preservation, enrichment and detection of the phosphoester amino acids (pSer, pThr and pTyr), pHis has remained invisible.

pHis is unique among phosphoamino acids in that two distinct, biologically relevant isomers occur. The imidazole side chain of His contains two nitrogen atoms (N1 and N3) that can both be phosphorylated to generate two biochemically distinct isomers; 1-phosphohistidine (1-pHis) or 3-phosphohistidine (3-pHis) (FIG. 1A) which are also referred to as tele-phosphohistidine (r-pHis) and pros-phosphohistidine (π-pHis) respectively (Attwood et al., Amino acids 32, 145 (January, 2007); McAllister et al., Biochemical Society transactions 41, 1072 (August, 2013)). NME1 and the closely related NME2 catalyze transfer of phosphate from ATP onto NDPs through a 1-pHis enzyme intermediate. The 3-pHis isomer has been shown to be more thermodynamically stable (Attwood et al., Amino acids 32, 145 (January, 2007)) than 1-pHis and may be more prevalent. 3-pHis is used by bacterial histidine kinases that autophosphorylate to initiate phosphotransfer cascades and it also plays an important role as an enzymatic intermediate for phospholipase D as well as several key metabolic enzymes including; phosphoglycerate mutase (PGAM), succinyl-CoA synthetase (SCS), ATP-citrate lyase (ACLY) (see, for example, Bond et al., J. Biol. Chem. 276, 3247 (2001)).

There is a need for the development of specific, monoclonal antibodies (mAbs) for detection of pHis that can be used to detect and functionally evaluate novel sites of protein phosphorylation. These antibodies can be used, for example, to investigate signal transduction pathways.

### SUMMARY

Uses of monoclonal antibodies, as well as antigen binding fragments thereof, are disclosed herein that specifically bind polypeptides including a histidine phosphorylated at N3 (3-pHis). In some embodiments, the antibody includes a heavy chain variable region and a light chain variable region, wherein the heavy chain variable region comprises a H-CDR1, a H-CDR2, and a H-CDR3, wherein the antibody or antigen binding fragment includes one of: a) the H-CDR1, the H-CDR2, and the H-CDR3 of the heavy chain variable region of the amino acid sequence set forth as SEQ ID NO: 1; b) the H-CDR1, the H-CDR2, and the H-CDR3 of the heavy chain variable region of the amino acid sequence set forth as SEQ ID NO: 2; c) the H-CDR1, the H-CDR2, and the H-CDR3 of the heavy chain variable region of the amino acid sequence set forth as SEQ ID NO: 3; or d) the H-CDR1, the H-CDR2, and the H-CDR3 of the heavy chain variable region of the amino acid sequence set forth as SEQ ID NO: 4, wherein the monoclonal antibody specifically binds a polypeptide comprising a histidine phosphorylated at N3 (3-pHis). In additional embodiments, the light chain variable region of the monoclonal antibody or antigen binding fragment includes a L-CDR1, a L-CDR2, and a L-CDR3, wherein the antibody or antigen binding fragment includes one of: a) the L-CDR1, the L-CDR2, and the L-CDR3 of the light chain variable region of the amino acid sequence set forth as SEQ ID NO: 5; b) the L-CDR1, the L-CDR2, and the L-CDR3 of the light chain variable region of the amino acid sequence set forth as SEQ ID NO: 6; c) the L-CDR1, the L-CDR2, and the L-CDR3 of the light chain variable region of the amino acid sequence set forth as SEQ ID NO: 7; or d) the L-CDR1, the L-CDR2, and the L-CDR3 of the light chain variable region of the amino acid sequence set forth as SEQ ID NO: 8.

In some embodiments, the antibodies include a heavy chain variable region and a light chain variable region, wherein the heavy chain variable region comprises a H-CDR1, a H-CDR2, and a H-CDR3, wherein: a) the H-CDR1, the H-CDR2, and the H-CDR3 comprise amino acids 21-28, 45-52, and 88-97 of SEQ ID NO: 1, respectively; b) the H-CDR1, the H-CDR2, and the H-CDR3 comprise amino acids 21-28, 46-52, and 91-101 of SEQ ID NO: 2, respectively; c) the H-CDR1, the H-CDR2, and the H-CDR3 comprise amino acids 24-31, 49-55, 94-104 of SEQ ID NO: 3, respectfully; or d) the H-CDR1, the H-CDR2, and the H-CDR3 comprise amino acids 24-31, 49-55, 94-104 of SEQ ID NO: 4, respectively. In additional embodiments, the light chain variable region comprises a L-CDR1, a L-CDR2, and a L-CDR3, wherein: a) the L-CDR1, the L-CDR2, and the L-CDR3 comprise amino acids 28-22, 51-53, and 90-102 of SEQ ID NO: 5, respectively; b) the L-CDR1, the L-CDR2, and the L-CDR3 comprise amino acids 27-34, 52-54, 91-103 of SEQ ID NO: 6, respectively; c) the L-CDR1, the L-CDR2, and the L-CDR3 comprise amino acids 27-34, 52-54, and 91-109 of SEQ ID NO: 7, respectively; or d) the L-CDR1, the L-CDR2, and the L-CDR3 comprise amino acids 27-33, 51-53 and 90-102 of SEQ ID NO: 8, respectively.

In further embodiments, methods are disclosed for using the antibodies, such as for detection of a polypeptide including a histidine phosphorylated at N3 (3-pHis). In some embodiments methods are disclosed for detecting the presence of a tumor and/or determining if a subject with a tumor will respond to a biologic or chemotherapeutic agent. In other embodiments, methods are disclosed for identifying an antibiotic.

The foregoing and other features of the disclosure will become more apparent from the following detailed description, which proceeds with reference to the accompanying figures.

### BRIEF DESCRIPTION OF THE DRAWINGS

FIGS. 1A-1D. Non-hydrolyzable phosphohistidine analogues and their incorporation into peptide libraries. (FIG. 1A) Structure of histidine and the two pHis isomers; 1-phosphohistidine (1-pHis) and 3-phosphohistidine (3-pHis). (FIG. 1B) Structures of the three synthetic peptide libraries used in this study in which either His or a stable pHis mimetic (1-pTza or 3-pTza) is flanked by randomized, neutral amino acids (alanine [A] and glycine [G)]). Each library is composed of 2⁸=256 unique peptides, is acylated at the N-terminus, amidated at the C-terminus, and contains L-cysteine (Cys) for chemical ligation to KLH (Ac-Cys.G/A.G/A.G/A.G/A.X.G/A.G/A.G/A.G/A-CONH2 (SEQ ID NO: 9)). (FIG. 1C) MS analysis was performed on all three of the peptide libraries. The results from analysis of the 3-pTza library is shown. (FIG. 1D) The peptide libraries were conjugated to the carrier protein keyhole limpet hemocyanin (KLH). Three rabbits were immunized with the 3-pTza library (7302, 7303 and 7304) and three rabbits were immunized with the 1-pTza library (7305, 7306 and 7307).

FIGS. 2A-2G. Dot blot screening of 3-pHis antisera and development of PGAM in vitro phosphorylation assays. (FIG. 2A) Dot blot screening of 3-pHis Antisera. (FIG. 2B) Crystal structure of PGAM co-crystallized with its phosphate donor 2,3-diphosphoglycerate (2,3-DPG). (FIG. 2C) GST-PGAM fusion protein was auto-phosphorylated in vitro by addition of increasing concentrations of 2,3-DPG. Reactions were stopped by addition of 5×pH 8.8 sample buffer and treated with or without heating to 95° C. for 10 min. Samples were analyzed immediately by a modified SDS-PAGE method in which gels were run and transferred at 4° C. and a pH 8.8 stacking gel was used. Immunoblotting with anti-sera from the three immunized rabbits (7302, 7303 and 7304) revealed that phospho-PGAM could only be detected by antisera from rabbit 7303. PGAM phosphorylation was abolished by heating the samples prior to SDS-PAGE. Mutation of the catalytic His residue (H11) also abolished phosphorylation of PGAM. (FIG. 2D) Purification of recombinant PGAM from E. coli. Crude E. coli lysates from cells pre-IPTG induction (lane 1) or 3 hr post-IPTG at 30° C. (lane 2) were run along-side purified PGAM (lane 3) after cleavage of the GST tag (FIG. 2E). Purified PGAM was auto-phosphorylated in vitro by incubation with 2,3-DPG for 10 min. at 30° C. Reactions were stopped by addition of 5×pH 8.8 sample buffer and treated with or without heating to 95° C. for 10 min. Samples were immediately analyzed by SDS-PAGE and immunoblotted with antisera from 7303 as in FIG. 2C. (FIG. 2F) 3-pHis isoform specificity. Recombinant NME1 and PGAM were auto-phosphorylated in vitro by incubation with ATP or 2,3-DPG respectively and blotted with 3-pHis antisera (7303). (FIG. 2G) Phospho-PGAM spot blots. In vitro phosphorylation of PGAM was performed as in FIG. 3E except reactions were stopped with addition of 2% SDS rather than sample buffer. Reactions were treated with or without heating to 95° C. for 10 min, diluted 1:5 and spotted directly on nitrocellulose instead of SDS-PAGE as a more rapid and convenient method that was developed to screen potential hybridoma clones for ability to produce anti-3-pHis antibodies. A representative immunoblot with anti-3-pHis (mAb MC39-4) is shown.

FIGS. 3A-3E. Affinity purification of anti-1-pHis and anti-3-pHis antibodies (FIG. 3A) Structures of the PEG-linker pTza peptide libraries used for affinity purification are shown covalently linked to agarose beads (Sulfolink coupling resin, Pierce) via a thioether bond with an N-terminal Cys residue. The agarose-linked pTza libraries were used in affinity columns to purify pHis antibodies from rabbit serum. (FIG. 3B) Fractions from the PEG-1-pTza affinity column including; input (IN), flowthrough (FT), washes (W1, W2, W3 and W4), 10 ul column material (col) and the elution fractions (E1 to E12) were analyzed by SDS-PAGE and Coomassie staining. (FIG. 3C) Western blotting of in vitro phosphorylated NME1 (5 or 200 ng) was performed with PEG-1-pTza column elution fractions E3 to E11 at a 1:200 dilution. (FIG. 3D) Fractions from the PEG-3-pTza affinity column were analyzed as described in B. (FIG. 3E) Western blotting of in vitro phosphorylated PGAM (5 or 200 ng) was performed with PEG-3-pTza column elution fractions E3 to E11 at a 1:200 dilution. PGAM is the only band visible.

FIGS. 4A-4F. Characterization of anti-1-pHis and anti-3-pHis antibodies using peptide dot blot arrays. Synthetic peptide dot blot arrays consisting of the His, 1-pTza or 3-pTza libraries (FIG. 1B), a pTyr (NCK) peptide and peptides with either His, 1-pTza or 3-pTza incorporated into nonapeptides of defined sequences (based on the pHis protein substrates; ACLY, NME1/2, histone H4, KCa3.1 and GNB1) were spotted on nitrocellulose and probed with; (FIG. 4A) affinity-purified polyclonal 3-pHis (7303-E6 (elution fraction #6)) or 1-pHis (7305-E6 (elution fraction #6)) antibodies or (FIG. 4B) anti-1-pHis mAbs (7305-SC1-1, SC50-3 and SC50-11). Peptide layouts, sequences and their sources are shown. (FIG. 4C) 3-pTza peptide dot blot characterization of anti-3-pHis mAbs. Peptide layouts, sequences and their sources are shown. A partially-deprotected, mono-ethyl ester version of the ACLY-based pTza peptide (AGAG-mono-Et-3-pTza-AGAG) was included. (FIGS. 4D-4F) Synthetic pTyr peptide dot blots. Peptides based on Nck, Eck and Fak were synthesized with or without a pTyr residue (there is no unphosphorylated Fak peptide) and spotted on nitrocellulose membranes in order to screen for pTyr crossreactivity of anti-pHis antibodies. Membranes were probed with; (FIG. 4D) anti-pTyr mAb 4G10, (FIG. 4E) anti-3-pHis mAbs (7303-MC39, 7304-MC44 and 7304-MC56) or (FIG. 4F) Anti-1-pHis mAbs (7305-SC1-1 and 7305-SC50-3). Anti-pHis mAbs were used at a concentration of 0.5 ug/ml. It was concluded that 3-pHis antibodies do not crossreact with pTyr.

FIGS. 5A-5C. Mammalian and cancer cell lysate blotting and pTyr cross-reactivity. (FIG. 5A) Src-transformed and non-transformed fibroblast cell lines (Psrc11 and pancreatic stellate cells (PaSCs) respectively) were analyzed by Western blotting. Cells were pre-treated with 1 mM ortho-vanadate for 30 min prior to lysis. The major 1-pHis (NME1/2) and 3-pHis (SCS and ACLY) bands detected are indicated. There is no detectable cross-reactivity of the pHis antibodies with pTyr. (FIG. 5B) A representative Western blot of pancreatic cancer cell line lysates with an anti-1-pHis mAb. (FIG. 5C) provides Western blot of the FLAG-NME1 stable 293 cells with an anti-3-pHis.

FIGS. 6A-6G. 3-pHis hybridoma subclone screening and antibody characterization. (FIG. 6A) High throughput, slot blot screening of 3-pHis hybridoma cell supernatants using lysates from E. coli transformed with GST-PGAM. The four best multiclonal (MC) anti-3-pHis hybridoma cell lines were identified (MC39, MC44, MC56 and MC60). Subcloning of these cell lines was performed to obtain anti-3pHis monoclonal cell lines. This resulted in 12 different, 3-pHis-positive subclones (SC) for MC39 (SC39-1 to -12) and MC44 (SC44-1 to -12), 3 different subclones for MC56 (SC56-2, -10 and -12) and 9 different subclones for MC60. Hybridoma cell supernatants from these subclones were normalized to 0.5 ug/mL IgG and incubated with PVDF membranes (using a BioRad slot blotting apparatus) transferred from preparative gels that were loaded with crude E. coli lysates. (FIG. 6B) Representative immunoblots blots from E. coli lysates, identical to those used in FIG. 6A, were treated with and without heating to 95° C. for 10 min. All of the detected bands were heat-sensitive, indicating the mAbs are 3-pHis specific and that there are many 3-pHis containing proteins present in the E. coli lysates. (FIGS. 6C-6E) Mammalian cell lysates were probed with an anti-3-pHis multiclonal mAb (MC44) that specifically recognized SCS in E. coli lysates but not PGAM. This mAb appears to have a sequence bias that is similar to the A/G peptide libraries used as immunogens. SCS is highly conserved from bacteria to humans. This mAb also detects the 3-pHis residue in ACLY (H760) and a number of other proteins in mammalian cell lysates. The amino acid sequences surrounding the pHis residues in ACLY, SCS and PGAM are shown for comparison. (FIG. 6F) A stable 293 cell line expressing FLAG-NME1 was blotted with the anti-3-pHis mAb SC39-5 and an anti-PGAM antibodies. SC39-5 detected a strong, heat sensitive band that corresponds to 3-pHis phospho-PGAM. NME1 was not detected, indicating the 3-pHis mAbs are isomer specific (i.e., do not cross-react with 1-pHis) (FIG. 6G). The same lysates from FIG. 6F were blotted with the anti-3-pHis mAb SC44-11 and a number of heat-sensitive bands are detected, including the known 3-pHis proteins SCS and ACLY.

FIGS. 7A-7B. Mass spectra and phosphorylation site assignment of in vitro phosphorylated NME1 and PGAM. (FIG. 7A) 5 ug of purified NME1 was incubated at RT with 1 mM ATP. (FIG. 7B) 5 ug of purified PGAM was incubated at 30° C. for 10 min with 1 mM 2,3-DPG.

FIGS. 8A-8B. Primary murine macrophages were isolated from bone marrow and fixed with paraformaldehyde. Macrophages were co-stained with the anti-3-pHis mAb SC39-4 (FIG. 8A) or anti-3-pHis mAb SC44-8 (FIG. 8B) and anti-ATP synthase antibodies (a mitochondrial enzyme) to check for co-localization with mitochondria.

FIGS. 9A-9D. Immunoaffinity purification using immobilized anti-1-pHis mAb SC1-1. A stably transfected HEK 293 cell line, FLAG-NME1, was used to prepare whole cell lysates for immunoaffinity purification of histidine phosphorylated proteins. (FIG. 9A). Immunoblotting of 1-pHis mAb column fractions was performed using 1-pHis mAb SC1-1 and anti-NME1/2 antibodies. Identical 1-pHis mAb column fractions were immunoblotted with; (FIG. 9B) 3-pHis mAbs SC39-4 and SC44-8 (FIG. 9C) anti-tubulin and anti-Rab5 antibodies, and (FIG. 9D) anti-3-pHis mAb SC56-2. The IN and FT fractions were treated with and without heating to 95° C. for 15 min. (Abbreviations: IN, input; FT, flow through; W1-W4, washes, E1-E6, elutions).

FIGS. 10A-10B. Immunoaffinity purification using immobilized anti-3-pHis mAb SC39-6. A stably transfected HEK 293 cell line, FLAG-NME1, was used to prepare whole cell lysates for immunoaffinity purification of histidine phosphorylated proteins. (FIG. 10A). Immunoblotting of 3-pHis mAb column fractions was performed using 3-pHis mAb SC39-6 and anti-PGAM antibodies. Identical 3-pHis mAb column fractions were immunoblotted with; (FIG. 10B) 3-pHis mAb SC44-8 and ACLY. The IN and FT fractions were treated with and without heating to 95° C. for 15 min. (Abbreviations: IN, input; FT, flow through; W1-W4, washes, E1-E6, elutions).

FIGS. 11A-11K. 1-pHis mAbs Negatively Stain Macrophage Phagosomes and 3-pHis mAbs Stain Centrosomes and Spindle Poles in HeLa Cells. (FIG. 11A) HeLa cells were fixed with PFA and stained with 1-pHis mAb SC1-1. White arrows indicate acidic compartments. (FIG. 11B) Macrophages were fed Dextran-AF488 and labeled with LysoTracker® for 60 min prior to fixation with PFA and staining with 1-pHis mAb SC1-1 was detected by Cy5 conjugated secondary antibodies. Bar, 10 μm. (FIG. 11C) Macrophages were incubated with Dextran-AF488 for 60 min and staining with mAb SC1-1 was detected by Cy5-conjugated secondary antibodies. (FIG. 11D) Macrophages were labeled with LysoTracker® for 60 min prior to fixation and mAb SC1-1 staining was detected by AF-488 conjugated secondary antibodies. (FIG. 11E) Co-staining of macrophages with mAb SC1-1 and Phalloidin-TRITC. (FIGS. 11F-11K) HeLa cells were fixed with; PFA (FIGS. 11F-11G), or pre-permeabilized with 0.5% Triton X-100 and fixed with PFA (FIGS. 11I and 11K) or methanol (FIGS. 11H and 11J) and stained with 3-pHis mAb SC39-4 alone (FIGS. 11F-11G) or co-stained with Aurora A (FIG. 11H), γ-tubulin (FIG. 11I) or α-tubulin (FIGS. 11J-11K) antibodies. (FIG. 11F) Metaphase cells are shown in an expanded view in the right panel. (FIG. 11G) From left to right, interphase, an early prophase and anaphase cells. (FIGS. 11H-11K) Cells in metaphase, prometaphase and telophase are shown. White arrows indicate centrosomes and spindle poles and grey arrows indicate midbodies in telophase cells. Nuclei were visualized with DAPI. Size Bar, 20 μm. See also FIG. 12.

FIGS. 12A-12S. pHis mAb Immunofluorescence Staining of Macrophages and HeLa Cells with Negative Controls. 3-pHis mAbs Stain Cytoplasmic and Nuclear Structures Distinct from 1-pHis mAbs, Related to FIG. 11. Macrophages were fixed with 4% PFA and co-stained with 3-pHis mAb SC39-4 and antibodies to the organelle markers LC3 (autophagosomes [FIG. 12A]) and Rab5 (early endosomes [FIG. 12B]). (FIGS. 12C-12E) U2OS cells were fixed with PFA co-stained with 3-pHis mAb SC39-4 and antibodies to γ-tubulin and α-tubulin. White arrows indicate centrosomes and spindle poles and grey arrows indicate midbodies in telophase cells. (FIGS. 12F-12J) 1-pHis mAb SC1-1 negative controls. Macrophages were stained with mAb SC1-1 that was pre-incubated with or without the immunizing peptide libraries for 30 min at RT with gentle agitation; (FIG. 12F) no peptide, (FIG. 12G) His peptide library, (FIG. 12H) 1-pTza peptide library or (FIG. 12I) 3-pTza peptide library. (J) Slides with PFA fixed macrophages were treated with or without boiling in citrate buffer for 10 min prior to staining with mAb SC1-1. (FIGS. 12K-12O) 3-pHis mAb SC39-4 negative controls. Macrophages were stained with mAb SC39-4 that was pre-incubated with or without the immunizing peptide libraries; (FIG. 12K) no peptide, (FIG. 12L) His peptide library, (FIG. 12M) 1-pTza peptide library or (FIG. 12N) 3-pTza peptide library. (FIG. 12O) Slides with PFA fixed macrophages were treated with or without boiling in citrate buffer for 10 min prior to staining with 3-pHis mAb SC39-4. (FIGS. 12P-12S) pTza blocking peptide negative controls in HeLa cells. HeLa cells were fixed with PFA and stained with mAb SC1-1 (FIGS. 12P-12Q) or mAb SC39-4 (FIGS. 12R-12S) that was pre-incubated with or without the 1-pTza or 3-pTza peptide libraries respectively for 30 min at RT with gentle agitation. Nuclei were visualized with DAPI. Size bars (FIGS. 12A-12O) 10 μm, (FIGS. 12P-12S) 20 μm.

### SEQUENCES

The nucleic and amino acid sequences listed are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [7158-93022-02_Sequence_Listing, Jul. 1, 2015, 39.1 KB], which is incorporated by reference herein.

The amino acid sequences for antibodies are provided below. In the following Fab fragment sequences (V_{H}-C_{H}1 or Vₖ-Cₖ1), the heavy and light chain variable domains (V_{H} and Vₖ) are underlined with the CDRs highlighted in bold. The heavy and light chain constant domains (C_{H}1 and Cₖ1) are in plain capital letters. Exemplary locations of the CDRs (as identified by IMGT) are listed below each sequence. The program available at www.IMGT.org was used to align the sequences and ID the CDRs.

SEQ ID NO: 1 is the amino acid sequence of the
heavy chain of mAb SC39-4.
ESGGRLVTPGGSLTLTCTVSGFSLSRYNMGWVRQAPGKGLEWIGW
IPFRGSLKYATWATGRFTISRTSTTVDLRMTGLTAADTATYFCVR
SSDGFDLWGPGTLVTVSSGQPKAPSVFPLAPCCGDTPSSTVTLGC
LVKGYLPEPVTVTWNSGTLTNGVRTFPSVRQSSGLYSLSSVVSVT
SSSQPVTCNVAHPATNTKVDKTV
CDR 1: 21-28; CDR2 45-52, CDR3 88-97; VH 1-108
SEQ ID NO: 2 is the amino acid sequence of the
heavy chain of mAb SC44-8.
ESGRGLVQPGGSLTLTCTASGFSIDSYGFSWVRQAPGKGLEHIGY
LTAGGRAFYASWAKSRSTITRNTNENTVTLKMTSLTAADTATYFC
AKLGSGNPVAIWGPGTLVTVSSGQPKAPSVFPLAPCCGDTPSSTV
TLGCLVKGYLPEPVTVTWNSGTLTNGVRTFPAVRESSGLYSLNSV
GKVTSSSQPVTCNVAHPATNTKVDKTV
CDR1 21-28; CDR2 46-52; CDR3 91-101; VH 1-112
SEQ ID NO: 3 is the amino acid sequence of the
heavy chain of mAb SC56-2.
SVKESEGGLIKPGGILTLTCTASGFSLSSYGFSWVRQAPGKGLEH
IGYLHANGRAYYATWAKSRSTITRNTNLNTVTLQLTSLTAADTAT
YFCAKIGSVSDVAIWGPGTLVTVSSGQPKAPSVFPLAPCCGDTPS
STVTLGCLVKGYLPEPVTVTWNSGTLTNGVRTFPSVRQSSGLYSL
SSVVSVTSSSQPVTCNVAHPATNTKVDKTV
CDR1 24-31; CDR2 49-55; CDR3 94-104, VH 1-115
SEQ ID NO: 4 is the amino acid sequence of the
heavy chain of mAb SC60-2
SVKESEGGLFKPTDTLTLTCTVSGFSLTTYGFSWVRQAPGKGLEW
IGYVRSDGRIYYTSWAKSRSTLTRNTNLNTVTLIMTSLTVADTAT
YFCAKIGSGTGVAIWGPGTLVTVSSGQPKAPSVFPLAPCCGDTPS
STVTLGCLVKGYLPEPVTVTWNSGTLTNGVRTFPSVRQSSGLYSL
SSVVSVTSSSQPVTCNVAHPATNTKVDKTV
CDR1 24-31; CDR2 49-55; CDR3 94-104; VH 1-115
SEQ ID NO: 5 is the amino acid sequence of the
light chain of mAb SC39-4.
AQFVMTQTPASVEAVVGGTVTIKCQASRDTGDGLIWYQQKPGQPP
KRLIYKASTVASGVPSRFKGRGSGTDFTLTISDLECADAATYYCH
SNFYNRWTYGNAFGGGTEVVVKGDPVAPTVLIFPPAADQVATGTV
TIVCVANKYFPDVTVTWEVDGTTQTTGIENSKTPQNSADCTYNLS
STLTLTSTQYNSHKEYTCKVTQGTTSVVQSFNRGDC
CDR1 28-33; CDR2 51-53; CDR3 90-102, VH 1-113
SEQ ID NO: 6 is the amino acid sequence of the
light chain of mAb SC44-8.
DPVMTPTPSFTSAAVGGTVTINCQSSQSVWRNKNLAWYQQKPGQP
PKRLIYAIATLDSGVPSRFSGSGSGTQFTLTISDVQCDDAATYYC
VGHYGSENDAYYAFGGGTEVVVKGDPVAPTVLIFPPSADLVATGT
VTIVCVANKYFPDVTVTWEVDGTTQTTGIENSKTPQNSADCTYNL
SSTLTLTSTQYNSHKEYTCKVTQGTTSVVQSFNRGDC
CDR1 27-34; CDR2 52-54; CDR3 91-103; VH 1-114
SEQ ID NO: 7 is the amino acid sequence of the
light chain of mAb SC56-2.
DPVMTQTPSSTSAAVGGTVTINCQSSESIYNNKNLAWYQQKPGQS
PRRLIYSISTLASGVSSRFKGSGSGTQFTLTISDVQCDDAATYYC
VGYYYSGGYYYSGSAAYYAFGGGTEVVVKGDPVAPTVLIFPPSAD
LVATGTVTIVCVANKYFPDVTVTWEVDGTTQTTGIENSKTPQNSA
DCTYNLSSTLTLTSTQYNSHKEYTCKVTQGTTSVVQSFNRGDC
CDR1 27-34: CDR2 52-54; CDR3 91-109: VH 1-120
SEQ ID NO: 8 is the amino acid sequence of the
light chain of mAb SC60-2.
DGVMTPTPASASAGVGGTVTINCQSSQSIYKKYIAWYQQKPGQPP
KRLIYSTSTLASGVSSRFKGSGSGTQFTLTISDVQCDDVATYYCV
GYYIITNDAYYSFGGGTEVVVKGDPVAPTVLIFPPSADLVATGTV
TIVCVANKYFPDVTVTWEVDGTTQTTGIENSKTPQNSADCTYNLS
STLTLTSTQYNSHKEYTCKVTQGTTSVVQSFNRGDC
CDR1 27-33; CDR2 51-53; CDR3 90-102; VH 1-113

SEQ ID NO: 9 is the amino acid sequence of a synthetic polypeptides.

SEQ ID NOs: 10-54 are the amino acid sequences of pHis substrates.

SEQ ID NOs: 55-59 are the amino acid sequences of synthetic polypeptides.

SEQ ID NOs: 60-88 are the nucleic acid sequences of primers.

SEQ ID NOs: 89-94 are the amino acid sequences of synthetic polypeptides.

### DETAILED DESCRIPTION

Stable pHis mimetics were incorporated into degenerate peptide libraries to immunize rabbits and develop the anti-3-pHis mAbs that constitute defined reagents with infinite supply. Several novel screening assays were developed to characterize these mAbs and it was demonstrated that they lack pTyr cross-reactivity and appear to detect pHis in a sequence-independent manner. Multiple rabbit hybridoma cell lines have been established for each pHis isomer and sequencing of the IgG heavy and light chain variable regions (V_{H} and V_{L}) revealed the distinct complementarity determining regions (CDRs). Antibodies were isolated that specifically bind polypeptides including a histidine phosphorylated at N3 (3-pHis). Nucleic acids encoding these antibodies, vectors including these nucleic acids, host cells transformed with these vectors are disclosed herein.

These antibodies are of use in detecting phosphorylation of polypeptides. In specific, non-limiting examples, the antibodies can be used to detect polypeptides phosphorylated in a signal transduction pathway.

### TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Unless otherwise explained, all technical and scientific terms used herein have the same meaning as commonly understood by one of ordinary skill in the art to which this disclosure belongs.

Administration: The introduction of a composition into a subject by a chosen route. Administration can be local or systemic. For example, if the chosen route is intravenous, the composition is administered by introducing the composition into a vein of the subject.

Agent: Any substance or any combination of substances that is useful for achieving an end or result; for example, a substance or combination of substances useful for inhibiting phosphorylation or for identifying the role of phosphorylation in a biological process. Agents include, and are not limited to, proteins, nucleic acid molecules, compounds, small molecules, organic compounds, inorganic compounds, or other molecules of interest. In some embodiments, the agent is a polypeptide agent (such as an antibody), or a pharmaceutical compound. The skilled artisan will understand that particular agents may be useful to achieve more than one result.

Amino acid substitution: The replacement of one amino acid in peptide with a different amino acid.

Amplification: A technique that increases the number of copies of a nucleic acid molecule (such as an RNA or DNA). An example of amplification is the polymerase chain reaction, in which a biological sample is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification can be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in PCT Publication No. WO 90/01069; ligase chain reaction amplification, as disclosed in European Patent Publication EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Animal: Living multi-cellular vertebrate organisms, a category that includes, for example, mammals and birds. The term mammal includes both human and non-human mammals. Similarly, the term “subject” includes both human and veterinary subjects.

Antibody: A polypeptide substantially encoded by an immunoglobulin gene or immunoglobulin genes, or antigen binding fragments thereof, which specifically binds and recognizes an analyte (antigen) such as one or more phosphorylated polypeptides, such as one or more polypeptides that includes a phosphorylated histidine, for example a histidine phosphorylated at N3. Immunoglobulin genes include the kappa, lambda, alpha, gamma, delta, epsilon and mu constant region genes, as well as the myriad immunoglobulin variable region genes.

Antibodies exist, for example, as intact immunoglobulins and as a number of well characterized fragments produced by digestion with various peptidases. Fabs, Fvs, scFvs that specifically bind to a phosphorylated polypeptide, such as a polypeptide that includes a phosphorylated histidine, for example a histidine phosphorylated at N3, are specific binding agents. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies and heteroconjugate antibodies such as bispecific antibodies. See also, Pierce Catalog and Handbook, 1994-1995 (Pierce Chemical Co., Rockford, Ill.); Kuby, Immunology, 3^{rd} Ed., W.H. Freeman & Co., New York, 1997.

Antibody fragments include, but are not limited to, the following: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule.

Antigen binding fragments of an antibody can be produced by the modification of whole antibodies or those synthesized de novo using recombinant DNA methodologies. In some examples, the term antibody includes the amino acid sequences of one or more of the CDRs from the antibody grafted onto a scaffold.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. The disclosed antibodies can be class switched.

Each heavy and light chain contains a constant region and a variable region, (the regions are also known as “domains”). In several embodiments, the heavy and the light chain variable domains combine to specifically bind the antigen. In additional embodiments, only the heavy chain variable domain is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). Light and heavy chain variable domains contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for antigen binding. The extent of the framework region and CDRs have been defined (see, Kabat et al., Sequences of Proteins of Immunological Interest, U.S. Department of Health and Human Services, 1991, which is hereby incorporated by reference in its entirety). The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus “CDR-H1”, as used herein, comprises residues 26 to 35, as described by a combination of the Kabat numbering system and Chothia's topological loop definition. The CDRs of the light chain variable domain are located at residues 24-34 (CDR-L1), residues 50-56 (CDR-L2) and residues 89-97 (CDR-L3) according to the Kabat numbering system. Lefranc, et al. (“IMGT unique numbering for immunoglobulin and T cell receptor variable domains and Ig superfamily V-like domains,” Dev. Comp. Immunol., 27:55-77, 2003) discloses the “IMGT” numbering scheme for CDRs. The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs of each chain are typically referred to as CDR1, CDR2, and CDR3 (from the N-terminus to C-terminus), and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Light chain CDRs are sometimes referred to as CDR L1, CDR L2, and CDR L3. Heavy chain CDRs are sometimes referred to as CDR H1, CDR H2, and CDR H3.

References to “V_{H}” or “VH” refer to the variable region of an immunoglobulin heavy chain, including that of an antibody fragment, such as Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected, or by a single cloned immunoglobulin. Monoclonal antibodies are produced by methods known to those of skill in the art, for instance by making hybrid antibody-forming cells from a fusion of myeloma cells with immune spleen cells. These fused cells and their progeny are termed “hybridomas.” In some embodiments, monoclonal antibodies can be humanized monoclonal antibodies. In some embodiments, monoclonal antibodies can be chimeric antibodies. In some examples monoclonal antibodies are isolated from a subject. The amino acid sequences of such isolated monoclonal antibodies can be determined.

A “humanized” antibody is an antibody including a human framework region and one or more CDRs from a non-human (such as a chimpanzee, mouse, rat, or synthetic) immunoglobulin. The non-human antibody providing the CDRs is termed a “donor,” and the human antibody providing the framework is termed an “acceptor.” In one embodiment, all the CDRs are from the donor antibody in a humanized antibody. Constant regions need not be present, but if they are, they must be substantially identical to human antibody constant regions, such as at least about 85-90%, such as about 95% or more identical. Hence, all parts of a humanized antibody, except possibly the CDRs, are substantially identical to corresponding parts of natural human antibody sequences. A “humanized antibody” can include a humanized light chain and a humanized heavy chain. A humanized antibody binds to the same antigen as the donor antibody that provides the CDRs. The acceptor framework of a humanized antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (for example, see U.S. Pat. No. 5,585,089).

A “chimeric” antibody is an antibody which includes sequences from two different antibodies, which typically are of different species. In some examples, a chimeric antibody includes one or more CDRs and/or framework regions from one chimpanzee antibody and CDRs and/or framework regions from another chimpanzee antibody. In some embodiments, a chimeric antibody comprises heavy and light chain variable regions derived from a first species and heavy and light chain constant regions derived from a second species. In some embodiments, the variable and constant regions of the light chain are derived from a first species while the variable region of the heavy chain is derived from the first species and the constant region of the heavy chain is derived from a second species. In some embodiments, the first species is non-human and includes, but is not limited to, a rabbit. In additional embodiments, the second species includes, but is not limited to, humans, non-human primate, mouse or rat.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. An epitope can be phosphorylated. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, antigens include peptides derived from a pathogen of interest or a cell type of interest, such as a tumor cell. Exemplary pathogens include bacteria, fungi, viruses and parasites. In some embodiments, an antigen is a phosphorylated polypeptide.

A “target epitope” is a specific epitope on an antigen that specifically binds an antibody of interest, such as a monoclonal antibody. In some examples, a target epitope includes the amino acid residues that contact the antibody of interest, such that the target epitope can be selected by the amino acid residues determined to be in contact with the antibody. In some embodiments, the target epitope includes a phosphorylated histidine.

Apoptotic cells: Non-dividing, non-viable cells that can be distinguished from necrotic cells (other dead cells). Apoptosis is a result of programmed cell death. According to characteristic morphological and biochemical features, apoptosis is characterized by shrinkage of the cell, dramatic reorganization of the cell nucleus, cell membrane and cell metabolism, active membrane blebbing, and ultimate fragmentation of the cell into membrane-enclosed vesicles (apoptotic bodies). The nuclear events of apoptosis begin with collapse of the chromatin against the nuclear periphery and into one or a few large clumps within the nucleus. Nuclear features include chromatin aggregation followed by DNA fragmentation (a specific marker of apoptotic process) after activation of endonucleases resulting in multiples subunits of DNA of an approximately 180 base pairs. The cellular events include cytoplasmic condensation and partition of the cytoplasm and nucleus into membrane bound-vesicles which contain ribosomes, intact mitochondria and nuclear material which are surrounded by an intact cellular membrane (a specific marker of apoptotic process when compared with necrosis, the other non physiological cell death process).

Binding affinity: Affinity of an antibody or antigen binding fragment thereof for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by a competition radioimmunoassay. In several examples, a high binding affinity is at least about 1×10⁻⁸ M. In other embodiments, a high binding affinity is at least about 1.5×10⁻⁸, at least about 2.0×10⁻⁸, at least about 2.5×10⁻⁸, at least about 3.0×10⁻⁸, at least about 3.5×10⁻⁸, at least about 4.0×10⁻⁸, at least about 4.5×10⁻⁸, or at least about 5.0×10⁻⁸ M.

Cancer: A malignant neoplasm that has undergone characteristic anaplasia with loss of differentiation, increase rate of growth, invasion of surrounding tissue, and is capable of metastasis. For example, thyroid cancer is a malignant neoplasm that arises in or from thyroid tissue, and breast cancer is a malignant neoplasm that arises in or from breast tissue (such as a ductal carcinoma). Residual cancer is cancer that remains in a subject after any form of treatment given to the subject to reduce or eradicate thyroid cancer. Metastatic cancer is a cancer at one or more sites in the body other than the site of origin of the original (primary) cancer from which the metastatic cancer is derived. Cancer includes, but is not limited to, solid tumors and hematologic malignancies.

Chemotherapy; chemotherapeutic agents: As used herein, any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating neoplasms such as solid tumors. In one embodiment, a chemotherapeutic agent is radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L., Berkery R. (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Chemotherapeutic agents include those known by those skilled in the art, including but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cis-platinum and the taxanes, such as taxol. Rapamycin has also been used as a chemotherapeutic.

Chemotherapy includes treatment with biological molecules such as cytokines, for example, an interleukin (IL), such as IL-2, or another factor, such as a tumor necrosis factor (TNF). Chemotherapy can also include treatment with nucleic acids, such as immunostimulatory nucleic acids, see for example, PCT Publication No. WO 2011/109422.

Clonal variant: Any sequence, which differs by one or more nucleotides or amino acids, in presence of V region with identical mutations compared to the germline, identical VDJ or VJ gene usage, and identical D and J length. The “germline” sequence is intended to be the sequence coding for the antibody/immunoglobulin (or of any fragment thereof) deprived of mutations, for example somatic mutations. The percentage of homology represents an indication of the mutational events which any type of heavy chain portion undergoes after contact with an antigen.

Cognate Response Regulator: A component of a two-component signal transduction system. The response regulator usually has a two-domain structure, with a conserved N-terminal regulatory domain, also called a receiver domain, and a variable C-terminal effector domain. The regulatory domain contains a conserved aspartate residue that receives the phosphoryl group from a phosphorylated histidine kinase. The phosphorylation of the regulatory domain brings about the conformational change, which leads to the activation of the effector domain that usually functions as the DNA-binding domain. Depending on the presence or absence of an environmental signal, the activity of a histidine kinase is regulated, and this in turn controls the cellular abundance of the phosphorylated response regulator that forms the phosphorelay couple with the histidine kinase.

Computer readable media: Any medium or media, which can be read and accessed directly by a computer, so that the media is suitable for use in a computer system. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

Conjugate: A complex of two molecules linked together, for example, linked together by a covalent bond. In one embodiment, an antibody is linked to an effector molecule; for example, an antibody that specifically binds to a phosphorylated polypeptide, such as a polypeptide that includes a phosphorylated histidine, for example a histidine phosphorylated at N3, covalently linked to an effector molecule or to a label. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.” In one embodiment, an antibody linked to an effector molecule or label is further joined to a lipid or other molecule to a protein or peptide to increase its half-life in the body.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a sample obtained from a healthy patient. In other embodiments, the control is a tissue sample obtained from a patient diagnosed with a disease of interest, such as cancer, that serves as a positive control. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. Suitable statistical analyses are well known in the art, and include, but are not limited to, Student's T test and ANOVA assays. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 100%, at least about 150%, at least about 200%, at least about 250%, at least about 300%, at least about 350%, at least about 400%, at least about 500%, or greater than 500%.

Cytokine: Proteins made by cells that affect the behavior of other cells, such as lymphocytes. In one embodiment, a cytokine is a chemokine, a molecule that affects cellular trafficking. Specific non-limiting examples of cytokines are IL-2, IFNγ, IL-6, and IL-10.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Non-limiting examples of detectable markers include fluorophores, fluorescent proteins, chemiluminescent agents, enzymatic linkages, radioactive isotopes and heavy metals or compounds (for example super paramagnetic iron oxide nanocrystals for detection by MRI). In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionuclides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance. Methods for using detectable markers and guidance in the choice of detectable markers appropriate for various purposes are discussed for example in Sambrook et al. (Molecular Cloning: A Laboratory Manual, Cold Spring Harbor, N. Y., 1989) and Ausubel et al. (In Current Protocols in Molecular Biology, John Wiley & Sons, New York, 1998).

Detecting, Determining or Measuring: To identify the existence, presence, or fact of something. General methods of detecting are known to the skilled artisan (see, for example, U.S. Pat. No. 7,635,476) and may be supplemented with the protocols and reagents disclosed herein. For example, included herein are methods of detecting a cell that expresses a phosphorylated polypeptide, such as a polypeptide that includes a phosphorylated histidine, for example a histidine phosphorylated at N3. These terms refer to measuring a quantity or quantitating a target molecule in the sample, either absolutely or relatively. Generally, detecting, measuring or determining a biological molecule requires performing an assay, such as mass spectrometry, and not simple observation.

Diagnostic: Identifying the presence or nature of a pathologic condition. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (for example severity) of a pathologic condition. In some examples prognostic is the probability that a subject will respond favorably to a treatment agent.

Effector molecule: The portion of a chimeric molecule that is intended to have a desired effect on a cell to which the chimeric molecule is targeted. Effector molecule is also known as an effector moiety, therapeutic agent, or diagnostic agent, or similar terms.

Framework Region: Amino acid sequences interposed between CDRs. The term includes variable light and variable heavy framework regions. The framework regions serve to hold the CDRs in an appropriate orientation for antigen binding.

Fc polypeptide: The polypeptide comprising the constant region of an antibody excluding the first constant region immunoglobulin domain. Fc region generally refers to the last two constant region immunoglobulin domains of IgA, IgD, and IgG, and the last three constant region immunoglobulin domains of IgE and IgM. An Fc region may also include part or all of the flexible hinge N-terminal to these domains. For IgA and IgM, an Fc region may or may not comprise the tailpiece, and may or may not be bound by the J chain. For IgG, the Fc region comprises immunoglobulin domains Cgamma2 and Cgamma3 (Cγ2 and Cγ3) and the lower part of the hinge between Cgamma1 (Cγ1) and Cγ2. Although the boundaries of the Fc region may vary, the human IgG heavy chain Fc region is usually defined to comprise residues C226 or P230 to its carboxyl-terminus, wherein the numbering is according to the EU index as in Kabat. For IgA, the Fc region comprises immunoglobulin domains Calpha2 and Calpha3 (Cα2 and Cα3) and the lower part of the hinge between Calpha1 (Cα1) and Cα2. Encompassed within the definition of the Fc region are functionally equivalent analogs and variants of the Fc region. A functionally equivalent analog of the Fc region may be a variant Fc region, comprising one or more amino acid modifications relative to the wild-type or naturally existing Fc region. Variant Fc regions will possess at least 50% homology with a naturally existing Fc region, such as about 80%, and about 90%, or at least about 95% homology. Functionally equivalent analogs of the Fc region may comprise one or more amino acid residues added to or deleted from the N- or C-termini of the protein, such as no more than 30 or no more than 10 additions and/or deletions. Functionally equivalent analogs of the Fc region include Fc regions operably linked to a fusion partner. Functionally equivalent analogs of the Fc region must comprise the majority of all of the Ig domains that compose Fc region as defined above; for example IgG and IgA Fc regions as defined herein must comprise the majority of the sequence encoding CH₂ and the majority of the sequence encoding CH₃. Thus, the CH₂ domain on its own, or the CH₃ domain on its own, are not considered Fc region. The Fc region may refer to this region in isolation, or this region in the context of an Fc fusion polypeptide.

Fluorophore: A chemical compound, which when excited by exposure to a particular stimulus, such as a defined wavelength of light, emits light (fluoresces), for example at a different wavelength (such as a longer wavelength of light).

Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules (such as aequorin) can eliminate the need for an external source of electromagnetic radiation, such as a laser.

Examples of particular fluorophores that can be used in the methods and for attachment to antibodies are provided in U.S. Pat. No. 5,866,366 to Nazarenko et al., such as 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives; LightCycler Red 640; Cy5.5; and Cy56-carboxyfluorescein; 5-carboxyfluorescein (5-FAM); boron dipyrromethene difluoride (BODIPY); N,N,N′,N′-tetramethyl-6-carboxyrhodamine (TAMRA); acridine, stilbene, -6-carboxy-fluorescein (HEX), TET (Tetramethyl fluorescein), 6-carboxy-X-rhodamine (ROX), Texas Red, 2′,7′-dimethoxy-4′,5′-dichloro-6-carboxyfluorescein (JOE), Cy3, Cy5, VIC® (Applied Biosystems), LC Red 640, LC Red 705, Yakima yellow amongst others.

Other suitable fluorophores include those known to those skilled in the art, for example those available from Molecular Probes (Eugene, Oreg.). In particular examples, a fluorophore is used as a donor fluorophore or as an acceptor fluorophore.

Gram-positive bacteria: Bacteria that stain dark blue or violet during Gram staining, and have a thick peptidoglycan layer. Exemplary Gram-positive bacteria that can be used in the disclosed methods include:

Actinobacteria
Actinomyces
Actinomyces israelii
Bacillales
Bacillus
Clostridium
Clostridium acetobutylicum
Clostridium aerotolerans
Clostridium argentinense
Clostridium baratii
Clostridium beijerinckii
Clostridium bifermentans
Clostridium botulinum
Clostridium butyricum
Clostridium cadaveris
Clostridium cellulolyticum
Clostridium chauvoei
Clostridium clostridioforme
Clostridium colicanis
Clostridium difficile
Clostridium estertheticum
Clostridium fallax
Clostridium formicaceticum
Clostridium histolyticum
Clostridium innocuum
Clostridium kluyveri
Clostridium ljungdahlii
Clostridium novyi
Clostridium paraputrificum
Clostridium perfringens
Clostridium phytofermentans
Clostridium piliforme
Clostridium ragsdalei
Clostridium ramosum
Clostridium septicum
Clostridium sordellii
Clostridium sporogenes
Clostridium sticklandii
Clostridium tertium
Clostridium tetani
Clostridium thermosaccharolyticum
Clostridium tyrobutyricum
Corynebacterium
Corynebacterium bovis
Corynebacterium diphtheriae
Corynebacterium granulosum
Corynebacterium jeikeium
Corynebacterium minutissimum
Corynebacterium renale
Enterococcus
Lactobacillales
Listeria
Nocardia
Nocardia asteroides
Nocardia brasiliensis
Propionibacterium acnes
Rhodococcus equi
Sarcina
Solobacterium moorei
Staphylococcus
Staphylococcus aureus
Staphylococcus capitis
Staphylococcus caprae
Staphylococcus epidermidis
Staphylococcus haemolyticus
Staphylococcus hominis
Staphylococcus lugdunensis
Staphylococcus muscae
Staphylococcus nepalensis
Staphylococcus pettenkoferi
Staphylococcus saprophyticus
Staphylococcus succinus
Staphylococcus warneri
Staphylococcus xylosus
Strangles
Streptococcus
Streptococcus agalactiae
Streptococcus anginosus
Streptococcus bovis
Streptococcus canis
Streptococcus iniae
Streptococcus lactarius
Streptococcus mitis
Streptococcus mutans
Streptococcus oralis
Streptococcus parasanguinis
Streptococcus peroris
Streptococcus pneumoniae
Streptococcus pyogenes
Streptococcus ratti
Streptococcus salivarius
Streptococcus sanguinis
Streptococcus sobrinus
Streptococcus suis
Streptococcus salivarius thermophilus
Streptococcus uberis
Streptococcus vestibularis
Streptococcus viridans

Gram-negative bacteria: Bacteria that loose or do not retain dark blue or violet stain during Gram staining, but instead are colored by a counterstain, such as safranin, and appear pink or ed. Gram-negative bacteria have a thin peptidoglycan layer. Exemplary Gram-negative bacteria that can be used in the disclosed methods include:

| | |
|---|---|
| Acetic acid bacteria | Fusobacterium necrophorum |
| Acinetobacter baumannii | Fusobacterium nucleatum |
| Agrobacterium tumefaciens | Fusobacterium polymorphum |
| Anaerobiospirillum | Haemophilus haemolyticus |
| Bacteroides | Haemophilus influenzae |
| Bacteroides fragilis | Helicobacter |
| Bdellovibrio | Helicobacter pylori |
| Brachyspira | Klebsiella pneumoniae |
| Cardiobacterium hominis | Legionella |
| Coxiella burnetii | Legionella pneumophila |
| Cyanobacteria | Leptotrichia buccalis |
| Cytophaga | Megamonas |
| Dialister | Megasphaera |
| Enterobacter | Moraxella |
| Enterobacter cloacae | Moraxella bovis |
| Enterobacteriaceae | Moraxella catarrhalis |
| Escherichia | Moraxella osloensis |
| Escherichia coli | Morganella morganii |
| Pseudomonas genome database | Negativicutes |
| Rickettsia rickettsii | Neisseria gonorrhoeae |
| Salmonella | Neisseria meningitidis |
| Salmonella enterica | Neisseria sicca |
| Salmonella enterica enterica | Pectinatus |
| Selenomonadales | Propionispora |
| Serratia marcescens | Proteobacteria |
| Shigella | Proteus mirabilis |
| Spirochaeta | Proteus penneri |
| Spirochaetaceae | Pseudomonas |
| Sporomusa | Pseudomonas aeruginosa |
| Stenotrophomonas | |
| Streptococcus gordonii | |
| Vampirococcus | |
| Verminephrobacter | |
| Vibrio cholerae | |
| Wolbachia | |
| Zymophilus | |

Histidine kinase: An enzyme that phosphorylates histidine, using the reaction:

ATP+polypeptide L-histidine⇄ADP+polypeptide N-phospho-L-histidine.

In this reaction, the two substrates are ATP and a polypeptide including a histidine, and the two products are ADP and a polypeptide including N-phospho-L-histidine, with phosphate linked either to the N1 or the N3 position. Histidine kinases are present, for example, in bacterial cells and mammalian cells.

A large family of histidine kinases and downstream signaling proteins, known as two-component regulatory systems, are widely employed by bacteria to link extracellular signals with transcription and chemotaxis. A bacterial histidine kinase is composed of several domains starting with a short N-terminal cytoplasmic portion connected to an extracellular sensing domain via a transmembrane α helix. A second transmembrane α helix connects the extracellular domain to the C-terminal cytoplasmic catalytic domain. Histidine kinases are known to serve roles in many different signal transduction pathways. The cytoplasmic domain tends to have high sequence homology and contains several well-known motifs, including the H, N, G1, F, and G2 boxes, which the extracellular sensing domain is not well conserved. Several crystal structures of a histidine kinase are available, see PDB Accession Nos. 1P0Z, 2CMN, 2GJ3, 2HJE, 2J48, 2O9B, 2O9C, 2R78, and 2R8R.

Similar phosphotransfer cascades function in plants to regulate processes such as ripening and circadian rhythms (Matthews, 1995). Although pHis signaling is commonly used in bacteria, the question remains whether or not pHis plays a role in eukaryotic signaling. There is growing evidence implicating putative mammalian histidine kinases NME1 and NME2 in cancer and tumor metastasis (Andolfo et al., 2011; Boissan et al., 2010; Thakur et al., 2011; Tso et al., 2013; Zhao et al., 2013). In fact, NME1 (AKA Nm23-H1 or nucleoside diphosphate kinase [NDPK]) was the first candidate metastasis suppressor gene identified (Steeg et al., 1988).

Host cells: Cells in which a vector can be propagated and its DNA expressed, for example a disclosed antibody can be expressed in a host cell. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgG: A polypeptide belonging to the class or isotype of antibodies that are substantially encoded by a recognized immunoglobulin gamma gene. In humans, this class comprises IgG₁, IgG₂, IgG₃, and IgG₄. In mice, this class comprises IgG₁, IgG₂a, IgG₂b, IgG₃.

Immune complex: The binding of antibody to a soluble antigen forms an immune complex. The formation of an immune complex can be detected through conventional methods known to the skilled artisan, for instance immunohistochemistry, immunoprecipitation, flow cytometry, immunofluorescence microscopy, ELISA, immunoblotting (for example, Western blot), magnetic resonance imaging, CT scans, X-ray and affinity chromatography. Immunological binding properties of selected antibodies may be quantified using methods well known in the art.

Immunoadhesin: A molecular fusion of a protein with the Fc region of an immunoglobulin, wherein the immunoglobulin retains specific properties, such as Fc receptor binding and increased half-life. An Fc fusion combines the Fc region of an immunoglobulin with a fusion partner, which in general can be any protein, polypeptide, peptide, or small molecule. In one example, an immunoadhesin includes the hinge, CH₂, and CH₃ domains of the immunoglobulin gamma 1 heavy chain constant region. In another example, the immunoadhesin includes the CH₂, and CH₃ domains of an IgG.

Immunoassay: A biochemical test that measures the presence or concentration of a substance in a sample, such as a biological sample, for example a serum sample obtained from a subject, using the reaction of an antibody to its cognate antigen, for example the specific binding of an antibody to a as a polypeptide that includes a phosphorylated histidine, for example a histidine phosphorylated at N3. The presence of antigen and/or the amount of antigen present can be measured. The phosphorylation state of the antigen can also be measured. In some examples, the amount of a polypeptide that includes a phosphorylated histidine, for example a histidine phosphorylated at N3 is measured.

Measuring the quantity of antigen (such as a phosphorylated polypeptide) can be achieved by a variety of methods. One of the most common is to label either the antigen or antibody with a detectable label. In some examples an antibody that specifically binds a polypeptide that includes a phosphorylated histidine, for example a histidine phosphorylated at N3 is labeled. Methods for labeling and guidance in the choice of labels appropriate for various purposes are discussed for example in Sambrook et al. (Molecular Cloning: A Laboratory Manual, Cold Spring Harbor, N. Y., 1989) Ausubel et al. (In Current Protocols in Molecular Biology, John Wiley & Sons, New York, 1998), and Harlow & Lane, (Antibodies, A Laboratory Manual, Cold Spring Harbor Publications, New York, 1988).

Immunologically reactive conditions: Includes reference to conditions which allow an antibody raised against a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See Harlow & Lane, supra, for a description of immunoassay formats and conditions. The immunologically reactive conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

Isolated: An “isolated” biological component (such as a cell, for example a B-cell, a nucleic acid, peptide, protein, heavy chain domain or antibody) has been substantially separated, produced apart from, or purified away from other biological components in the cell of the organism in which the component naturally occurs, such as, other chromosomal and extrachromosomal DNA and RNA, and proteins. Nucleic acids and polypeptides which have been “isolated” thus include nucleic acids and proteins purified by standard purification methods. The term also embraces nucleic acids and polypeptides prepared by recombinant expression in a host cell as well as chemically synthesized nucleic acids. In some examples an antibody, such as an antibody that specifically binds phosphorylated polypeptide, such as a polypeptide that includes a phosphorylated histidine, for example a histidine phosphorylated at N3 can be isolated.

K_{d}: The dissociation constant for a given interaction, such as a polypeptide ligand interaction or an antibody antigen interaction. For example, for the bimolecular interaction of an antibody (such as any of the antibodies disclosed herein) and an antigen (such as phosphorylated polypeptide) it is the concentration of the individual components of the bimolecular interaction divided by the concentration of the complex.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In some examples, a disclosed antibody is labeled.

Mass Spectrometry: A process used to separate and identify molecules based on their mass. Mass spectrometry ionizes chemical compounds to generate charged molecules or molecule fragments and measures their mass-to-charge ratios. In a typical MS procedure, as sample is ionized. The ions are separated according to their mass-to-charge ratio, and the ions are dynamically detected by some mechanism capable of detecting energetic charged particles. The signal is processed into the spectra of the masses of the particles of that sample. The elements or molecules are identified by correlating known masses by the identified masses. “Time-of-flight mass spectrometry” (TOFMS) is a method of mass spectrometry in which an ion's mass-to-charge ratio is determined via a time measurement. Ions are accelerated by an electric field of known strength. This acceleration results in an ion having the same kinetic energy as any other ion that has the same charge. The velocity of the ion depends on the mass-to-charge ratio. The time that it subsequently takes for the particle to reach a detector at a known distance is measured. This time will depend on the mass-to-charge ratio of the particle (heavier particles reach lower speeds). From this time and the known experimental parameters one can find the mass-to-charge ratio of the ion. “Liquid chromatography-mass spectrometry” or “LC-MS” is a chemistry technique that combines the physical separation capabilities of liquid chromatography (or HPLC) with the mass analysis capabilities of mass spectrometry. Liquid chromatography mass spectrometry (LC-MS) separates compounds chromatographically before they are introduced to the ion source and mass spectrometer. It differs from gas chromatography (GC-MS) in that the mobile phase is liquid, usually a mixture of water and organic solvents, instead of gas and the ions fragments. Most commonly, an electrospray ionization source is used in LC-MS.

Mean and Standard Deviation: The arithmetic mean is the “standard” average, often simply called the “mean”.

$$\overline{x} = \frac{1}{n} \cdot {\sum }_{i = 1}^{n} {x}_{i}$$

The mean is the arithmetic average of a set of values.

The standard deviation (represented by the symbol sigma, σ) shows how much variation or “dispersion” exists from the mean. The standard deviation of a random variable, statistical population, data set, or probability distribution is the square root of its variance. The standard deviation is commonly used to measure confidence in statistical conclusions. Generally, twice the standard deviation is about the radius of a 95 percent confidence interval. Effects that fall far outside the range of standard deviation are generally considered statistically significant. One of skill in the art can readily calculate the mean and the standard deviation from a population of values.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989). An oligonucleotide is a linear polynucleotide sequence of up to about 100 nucleotide bases in length.

ClustalW is a program that aligns three or more sequences in a computationally efficient manner. Aligning multiple sequences highlights areas of similarity which may be associated with specific features that have been more highly conserved than other regions. Thus, this program can classify sequences for phylogenetic analysis, which aims to model the substitutions that have occurred over evolution and derive the evolutionary relationships between sequences. The ClustalW multiple sequence alignment web form is available on the internet from EMBL-EBI (ebi.ac.uk/Tools/msa/clustalw2/), see also Larkin et al., Bioinformatics 2007 23(21): 2947-2948.

A polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the antibodies herein disclosed.

In general, the nature of the carrier will depend on the particular mode of administration being employed. For instance, parenteral formulations usually comprise injectable fluids that include pharmaceutically and physiologically acceptable fluids, which include, but are not limited to, water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition capable of inducing a desired therapeutic or prophylactic effect when properly administered to a subject or a cell. In some examples a pharmaceutical agent includes one or more of the disclosed antibodies.

Phosphorylation: The addition of a phosphate (PO₄³⁻) group to a polypeptide or other organic molecule. Phosphorylation of proteins plays a significant role in a number of biological processes. The reversible phosphorylation of proteins is an important regulatory mechanism that occurs in both prokaryotic and eukaryotic organisms. In vivo, serine phosphorylation is the most common type of phosphorylation, followed by threonine phosphorylation. Tyrosine, aspartate and histidine are also phosphorylated. Phosphorylation of polypeptides can be detected by antibodies, electrophoresis, such as SDS-PAGE, and mass spectrometry.

Polypeptide: Any chain of amino acids, regardless of length or posttranslational modification (e.g., glycosylation or phosphorylation). In one embodiment, the polypeptide includes a phosphorylated histidine, for example a histidine phosphorylated at N3. In one embodiment, the polypeptide is a disclosed antibody or a fragment thereof.

A “residue” refers to an amino acid or amino acid mimetic incorporated in a polypeptide by an amide bond or amide bond mimetic. A polypeptide has an amino terminal (N-terminal) end and a carboxy terminal end. Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

A “polypeptide including a histidine phosphorylated” at N3 has one or more histidine residues phosphorylated at N3 in its amino acid sequence. The polypeptide can also have histidine residues phosphorylated at N1 and/or unphosphorylated histidines in its amino acid sequence, provided the polypeptide has at least one histidine phosphorylated at N3 is present in the amino acid sequence of the polypeptide. In some embodiments, a polypeptide including a histidine phosphorylated at N3, can have 1, 2, 3, 4, 5, 6, 7, 8, 9, 10 or more histidines phosphorylated at N3 in its amino acid sequence.

Preventing or treating a disease: “Preventing” a disease refers to inhibiting the full development of a disease, for example in a person who is known to have a predisposition to a disease such as a tumor. An example of a person with a known predisposition is someone with a history of breast cancer in the family, or who has been exposed to factors that predispose the subject to a condition, such as melanoma. “Treatment” refers to a therapeutic intervention that ameliorates a sign or symptom of a disease or pathological condition after it has begun to develop. In several embodiments, treatment refers to a reduction in the size or volume of a tumor, a decrease in the number and/or size of metastases, a decrease in a symptom of the tumor, or combinations thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50%, 60%, 70%, 80%, 90%, 95%, 96%, 97%, 98%, or 99% of the total peptide or protein content of the preparation.

Quantitating: Determining or measuring a quantity (such as a relative quantity) of a molecule or the activity of a molecule, such as the quantity of a polypeptide that includes phosphorylated histidine, for example a histidine phosphorylated at N3 present in a sample.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sample: A biological sample obtained from a subject, such as a human or other primate or mammal, which contains for example nucleic acids and/or proteins. As used herein, biological samples include all clinical samples that include polypeptides, such as those obtained from subjects, including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In particular embodiments, the biological sample is obtained from a subject, such as in the form of a blood sample, such as serum sample. In one example, the sample is a tumor sample.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of polypeptide sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet (along with a description of how to determine sequence identity using this program).

Homologs and variants of a V_{L} or a V_{H} of an antibody that specifically binds a polypeptide are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. One of skill in the art will appreciate that these sequence identity ranges are provided for guidance only; it is entirely possible that strongly significant homologs could be obtained that fall outside of the ranges provided. Thus, in some examples a heavy chain of an antibody or antigen binding fragment thereof has at least 90%, at least 91%, at least 92%, at least 93%, at least 94%, at least 95%, at least 96%, at least 97%, at least 98% or at least 99% sequence identity to any of SEQ ID NOS: 1, 2, 3, or 4, wherein the variant specifically binds a polypeptide phosphorylated at a histidine, specifically a histidine phosphorylated at N3. In some examples a light chain of an antibody or antigen binding fragment thereof has at least 90%, at least 91%, at least 92%, at least 93%, at least 94%, at least 95%, at least 96%, at least 97%, at least 98% or at least 99% sequence identity to any of SEQ ID NOS: 5, 6, 7 or 8, wherein the variant specifically binds a polypeptide phosphorylated at a histidine, specifically a histidine phosphorylated at N3.

Nucleic acids that “selectively hybridize” or “selectively bind” do so under moderately or highly stringent conditions that excludes non-related nucleotide sequences. In nucleic acid hybridization reactions, the conditions used to achieve a particular level of stringency will vary, depending on the nature of the nucleic acids being hybridized. For example, the length, degree of complementarity, nucleotide sequence composition (for example, GC v. AT content), and nucleic acid type (for example, RNA versus DNA) of the hybridizing regions of the nucleic acids can be considered in selecting hybridization conditions. An additional consideration is whether one of the nucleic acids is immobilized, for example, on a filter.

A specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N. Y., 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Signal Transduction: A process that occurs when an extracellular molecule activates a receptor on the surface of a cell. The receptor triggers biochemical events inside the cells, leading to a biological response. These biological responses can be, for example, changes in cell metabolism, phenotype, differentiation, proliferation, and/or gene expression. Signal transduction can involve phosphorylation of the receptor or polypeptides within the cell.

Specifically bind: When referring to an antibody, refers to a binding reaction which determines the presence of a target protein, peptide, or phosphorylated polypeptide in the presence of a heterogeneous population of proteins and other biologics. Thus, under designated conditions, an antibody binds preferentially to a particular target protein or peptide (such as phosphorylated form of the polypeptide, such as a polypeptide that includes a phosphorylated histidine, for example a histidine phosphorylated at N3) and do not bind in a significant amount to (1) other polypeptides or proteins present in the sample, does not bind the unphosphorylated form of the polypeptide, and/or does not bind the polypeptide including a different phosphorylated amino acid and/or does not bind the polypeptide include a histidine phosphorylated at N1/N2. In a specific example, an antibody that specifically binds a polypeptide comprising a histidine phosphorylated at N3 does not bind in a significant amount to other polypeptides or proteins present in the sample, the unphosphorylated form of the polypeptide, the polypeptide including a different phosphorylated amino acid and the polypeptide include a histidine phosphorylated at N1/N2. Specific binding can be determined by methods known in the art. With reference to an antibody antigen complex, specific binding of the antigen and antibody has a K_{d} of less than about 10⁻⁷ Molar, such as less than about 10⁻⁷ Molar, 10⁻⁸ Molar, 10⁻⁹ Molar, or even less than about 10⁻¹⁰ Molar.

Standard: A substance or solution of a substance of known amount, purity or concentration that is useful as a control. A standard can also be a known value or concentration of a particular substance. A standard can be compared (such as by spectrometric, chromatographic, spectrophotometric, or statistical analysis) to an unknown sample (of the same or similar substance) to determine the presence of the substance in the sample and/or determine the amount, purity or concentration of the unknown sample. In one embodiment, a standard can be the amount of a polypeptide including a histidine phosphorylated at N3 in a sample from a subject that does not have a particular condition, such as a tumor.

Therapeutic agent: Used in a generic sense, it includes treating agents, prophylactic agents, and replacement agents.

Therapeutically effective amount or effective amount: A quantity of a specific substance, such as an antibody, sufficient to achieve a desired effect in a subject being treated. In several embodiments, a therapeutically effective amount is the amount necessary to reduce a sign or symptom of a disorder. When administered to a subject, a dosage will generally be used that will achieve target tissue concentrations that has been shown to achieve a desired in vitro effect.

Tumor: An abnormal growth of cells, which can be benign or malignant. Cancer is a malignant tumor, which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

Under conditions sufficient for: A phrase that is used to describe any environment that permits the desired activity.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Monoclonal Antibodies that Specifically Bind Phosphorylated Histidine

Isolated monoclonal antibodies (mAb) and antigen binding fragments thereof are disclosed herein that specifically bind a polypeptide including a phosphorylated histidine, specifically a histidine phosphorylated at N3. In some embodiments, the monoclonal antibodies specifically bind a polypeptide including a histidine phosphorylated at N3 (3-pHis) with an equilibrium constant (K_{d}) of 1 nM or less. In several embodiments, the monoclonal antibodies and antigen binding fragments bind a polypeptide including a histidine phosphorylated at N3, with a binding affinity of 1×10⁻⁹ M, at least about 1.5×10⁻⁹M, at least about 2×10⁻⁹M, at least about 3×10⁻⁹ M, at least about 4×10⁻⁹ M, at least about 5×10⁻⁹M, at least about 6×10⁻⁹M, at least about 7×10⁻⁹ M, at least about 8×10⁻⁹M, at least about 9×10⁻⁹M, or at least about 1×10⁻¹⁰ M.

The structure of histidine is shown below (arrow shows the N3 position):

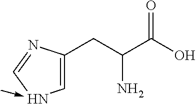

The monoclonal antibodies disclosed herein bind to a polypeptide including a histidine phosphorylated at N3 regardless of the amino acid sequence of the polypeptide. In some embodiments, the monoclonal antibodies disclosed herein can specifically bind to a polypeptide that includes a histidine phosphorylated at N3 that is present in any amino acid sequence. Thus, any amino acid sequence can be specifically bound by the mAb, provided the amino acid sequence includes histidine phosphorylated at N3.

In some embodiments, the monoclonal antibody can bind more than one polypeptide including a histidine phosphorylated at N3, wherein the amino acid sequences of the polypeptide differ. However, all the polypeptides must include a histidine phosphorylated at N3. Thus, the antibody specifically binds to these polypeptides. In specific examples, the amino acid sequence of the polypeptide is not critical for binding of the monoclonal antibody. In additional embodiments, the antibody specifically binds one or more polypeptides including a histidine phosphorylated at N3, but does not bind the polypeptides when histidine is not phosphorylated at N3.

In further embodiments, the monoclonal antibody can bind a polypeptide with a specified amino acid sequence (“X”) including a histidine phosphorylated at N3, but does not bind the polypeptide with the specified amino acid sequence (“X”) when the polypeptide does not include a histidine phosphorylated at N3. The monoclonal antibody can also bind a polypeptide with a different amino acid sequence (“Y”) including a histidine phosphorylated at N3, but does not bind the polypeptide with the specified amino acid sequence (“Y”) when the polypeptide does not include a histidine phosphorylated at N3.

The monoclonal antibody can be of any isotype. The monoclonal antibody can be, for example, an IgM or an IgG antibody, such as IgG₁ or an IgG₂. The class of an antibody that specifically binds a polypeptide phosphorylated at a histidine, specifically a histidine phosphorylated at N1 or N3, can be switched with another. In one aspect, a nucleic acid molecule encoding V_{L} or V_{H} is isolated using methods well-known in the art, such that it does not include any nucleic acid sequences encoding the constant region of the light or heavy chain, respectively. The nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that specifically binds a polypeptide phosphorylated at a histidine, specifically a histidine phosphorylated at N1 or N3 that was originally IgM may be class switched to an IgG. Class switching can be used to convert one IgG subclass to another, such as from IgG₁ to IgG₂.

The monoclonal antibodies disclosed herein can be rabbit antibodies and can include a rabbit framework region. In some embodiments, the monoclonal antibodies are humanized, and thus include one or more human framework regions. Exemplary framework regions are disclosed, for example, in PCT Publication No. WO 2011/038290 and U.S. Patent Application No. 2012/0244166A1, which are incorporated by reference herein. In some embodiments, the monoclonal antibodies disclosed herein are chimeric antibodies. In some embodiments, the monoclonal antibodies include rabbit and human regions.

In some embodiments, the monoclonal antibody includes both a heavy chain variable domain and a light chain variable domain. Naturally-occurring antibodies are immunoglobulin molecules comprised of four polypeptide chains, two heavy (H) chains and two light (L) chains inter-connected by disulfide bonds. Each heavy chain is comprised of a heavy chain variable region (VH) and a heavy chain constant region. The heavy chain constant region is comprised of three domains, CH1, CH2 and CH3. Each light chain is comprised of a light chain variable region (VL) and a light chain constant region. The light chain constant region is comprised of one domain, CL. The VH and VL regions can be further subdivided into regions of hypervariability, called complementarity determining regions (CDR), interspersed with regions that are more conserved, called framework regions (FWR). Each VH and VL is composed of three CDRs and four FWRs, arranged from amino-terminus to carboxy-terminus in the following order: FWR1, CDR1, FWR2, CDR2, FWR3, CDR3, FWR4.

In several embodiments, the monoclonal antibodies include a heavy chain comprising a heavy chain complementarity determining region (HCDR)1, a HCDR2 and an HCDR3, and a light chain comprising a light chain complementarity determining region (LCDR)1, LCDR2 and LCDR3. In some embodiments, the antibodies include a variable heavy (V_{H}) and a variable light (V_{L}) chain. In several embodiments, the antibody or antigen binding fragment thereof includes heavy and light chain variable regions including the HCDR1, HCDR2, and HCDR3, and LCDR1, LCDR2, and LCDR3, respectively, of one of the SC39-4, SC44-8, SC56-2, or SC60-2 antibodies.

The discussion of monoclonal antibodies below refers to isolated monoclonal antibodies that include heavy and light chain variable domains including at least one complementarity determining region (CDR), such as a CDR1, CDR2 and CDR3. The person of ordinary skill in the art will understand that various CDR numbering schemes (such as the Kabat, Chothia or IMGT numbering schemes) can be used to determine CDR positions. The amino acid sequence and the CDR positions of the heavy and light chain of the SC39-4, SC44-8, SC56-2 and SC60-2 monoclonal antibodies according to the IMGT are provided herein. The person of skill in the art will readily understand use of various CDR numbering schemes when referencing particular amino acids of the antibodies disclosed herein.

In some embodiments, disclosed is an isolated monoclonal antibody or antigen binding fragment thereof, including a heavy chain variable region and a light chain variable region, wherein the heavy chain variable region includes a H-CDR1, a H-CDR2, and a H-CDR3, wherein the antibody or antigen binding fragment includes: a) the H-CDR1, the H-CDR2, and the H-CDR3 of the heavy chain variable domain of the amino acid sequence set forth as SEQ ID NO: 1; b) the H-CDR1, the H-CDR2, and the H-CDR3 of the heavy chain variable domain of the amino acid sequence set forth as SEQ ID NO: 2; c) the H-CDR1, the H-CDR2, and the H-CDR3 of the heavy chain variable domain of the amino acid sequence set forth as SEQ ID NO: 3; or d) the H-CDR1, the H-CDR2, and the H-CDR3 of the heavy chain variable domain of the amino acid sequence set forth as SEQ ID NO: 4, wherein the monoclonal antibody specifically binds a polypeptide including a histidine phosphorylated at N3 (3-pHis). In additional embodiments, disclosed is an isolated monoclonal antibody or antigen binding fragment of claim 1, wherein the light chain variable domain includes a L-CDR1, a L-CDR2, and a L-CDR3, wherein the antibody or antigen binding fragment includes: a) the L-CDR1, the L-CDR2, and the L-CDR3 of the light chain variable domain of the amino acid sequence set forth as SEQ ID NO: 5; b) the L-CDR1, the L-CDR2, and the L-CDR3 of the light chain variable domain of the amino acid sequence set forth as SEQ ID NO: 6; c) the L-CDR1, the L-CDR2, and the L-CDR3 of the light chain variable domain of the amino acid sequence set forth as SEQ ID NO: 7; or d) the L-CDR1, the L-CDR2, and the L-CDR3 of the light chain variable domain of the amino acid sequence set forth as SEQ ID NO: 8. In additional embodiments, disclosed is an isolated monoclonal antibody or antigen binding fragment, including a) the H-CDR1, H-CDR2, and H-CDR3 of the amino acid sequence set forth as SEQ ID NO: 1, and the L-CDR1, L-CDR2, and L-CDR3 of the amino acid sequence set forth as SEQ ID NO: 5; b) the H-CDR1, H-CDR2, and H-CDR3 of the amino acid sequence set forth as SEQ ID NO: 2 and the L-CDR1, L-CDR2, and L-CDR3 of the amino acid sequence set forth as SEQ ID NO: 6; c) the H-CDR1, H-CDR2, and H-CDR3 of the amino acid sequence set forth as SEQ ID NO: 3 and the L-CDR1, L-CDR2, and L-CDR2 of the amino acid sequence set forth as SEQ ID NO: 7; or d) the H-CDR1, H-CDR2, and H-CDR3 of the amino acid sequence set forth as SEQ ID NO: 4, and the L-CDR1, L-CDR2, and L-CDR3 of the amino acid sequence set forth as SEQ ID NO: 8.

For example, in some embodiments, the antibody includes a heavy chain variable region including a HCDR1, HCDR2, and/or HCDR3 including amino acids 21-28, 45-52, and 88-97 of SEQ ID NO: 1, respectively. In further embodiments, the antibody includes a heavy chain variable region including a HCDR1, HCDR2, and/or HCDR3 including amino acids 21-28, 46-52, and 91-101 of SEQ ID NO: 2, respectively. In additional embodiments, the antibody includes a heavy chain variable region including a HCDR1, HCDR2, and/or HCDR3 including amino acids 24-31, 49-55, 94-104 of SEQ ID NO: 3, respectively. In more embodiments, the antibody includes a heavy chain variable region including a HCDR1, HCDR2, and/or HCDR3 including amino acids 24-31, 49-55, 94-104 of SEQ ID NO: 4, respectively. The antibody specifically binds a polypeptide comprising a histidine phosphorylated at N3.

In some embodiments, the antibody includes a light chain variable region including a LCDR1, LCDR2, and/or LCDR3 including amino acids 28-22, 51-53, and 90-102 of SEQ ID NO: 5, respectively. In further embodiments, the antibody includes a light chain variable region including a LCDR1, LCDR2, and/or LCDR3 including amino acids 27-34, 52-54, 91-103 of SEQ ID NO: 6, respectively. In additional embodiments, the antibody includes a light chain variable region including a LCDR1, LCDR2, and/or LCDR3 including amino acids 27-34, 52-54, and 91-109 of SEQ ID NO: 7, respectively. In more embodiments, the antibody includes a light chain variable region including a LCDR1, LCDR2, and/or LCDR3 including amino acids 27-33, 51-53 and 90-102 of SEQ ID NO: 8, respectively. The antibody specifically binds a polypeptide including a histidine phosphorylated at N3.

In some embodiments, the antibody includes a heavy chain variable region including a HCDR1, HCDR2, and HCDR3 including amino acids 28, 45-52, and 88-97 of SEQ ID NO: 1, respectively, and a light chain variable region including a LCDR1, LCDR2, and LCDR3 including amino acids 28-22, 51-53, and 90-102 of SEQ ID NO: 5 respectively. In additional embodiments, the antibody includes a heavy chain variable region including a HCDR1, HCDR2, and HCDR3 including amino acids 21-28, 46-52, and 91-101 of SEQ ID NO: 2, respectively, and a light chain variable region including a LCDR1, LCDR2, and LCDR3 including amino acids 27-34, 52-54, 91-103 of SEQ ID NO: 6, respectively. In further embodiments, the antibody includes a heavy chain variable region including a HCDR1, HCDR2, and HCDR3 including amino 24-31, 49-55, 94-104 of SEQ ID NO: 3, respectively, and a light chain variable region including a LCDR1, LCDR2, and LCDR3 including amino acids 27-34, 52-54, and 91-109 of SEQ ID NO: 7, respectively. In more embodiments, the antibody includes a heavy chain variable region including a HCDR1, HCDR2, and HCDR3 including amino acids 24-31, 49-55, 94-104 of SEQ ID NO: 4, respectively, and a light chain variable region including a LCDR1, LCDR2, and LCDR3 including amino acids 27-33, 51-53 and 90-102 of SEQ ID NO: 8, respectively.

In further embodiments, the antibody includes a heavy chain variable region including an amino acid sequence at least 95%, 96%, 97%, 98%, or 99% identical to the amino acid sequence set forth as one of a) amino acids 1-108 of SEQ ID NO: 1; b) amino acids 1-112 of SEQ ID NO: 2; c) amino acid 1-115 of SEQ ID NO: 3, or d) amino acids 1-115 of SEQ ID NO: 4. In more embodiments, the antibody includes a light chain variable region including an amino acid sequence at least 95%, 96%, 97%, 98%, or 99% identical to the amino acid sequence set forth as one of a) amino acids 1-113 of SEQ ID NO: 5; b) amino acids 1-114 of SEQ ID NO: 6; c) amino acid 1-120 of SEQ ID NO: 7; or d) amino acids 1-113 of SEQ ID NO: 8. Thus, the antibody can include a) a heavy chain variable region including an amino acid sequence at least 95%, 96%, 97%, 98%, or 99% identical to the amino acid sequence set forth as amino acids 1-108 of SEQ ID NO: 1 and a light chain variable region including an amino acid sequence at least 95%, 96%, 97%, 98%, or 99% identical to the amino acid sequence set forth as amino acids amino acids 1-113 of SEQ ID NO: 5; b) a heavy chain variable region including an amino acid sequence at least 95%, 96%, 97%, 98%, or 99% identical to the amino acid sequence set forth as amino acids 1-112 of SEQ ID NO: 2 and a light chain variable region including an amino acid sequence at least 95%, 96%, 97%, 98%, or 99% identical to the amino acid sequence set forth as amino acids amino acids 1-114 of SEQ ID NO: 6; c) a heavy chain variable region including an amino acid sequence at least 95%, 96%, 97%, 98%, or 99% identical to the amino acid sequence set forth as amino acids 1-115 of SEQ ID NO: 3 and a light chain variable region including an amino acid sequence at least 95%, 96%, 97%, 98%, or 99% identical to the amino acid sequence set forth as amino acids amino acids 1-120 of SEQ ID NO: 7; or d) a heavy chain variable region including an amino acid sequence at least 95%, 96%, 97%, 98%, or 99% identical to the amino acid sequence set forth as amino acids 1-115 of SEQ ID NO: 4 and a light chain variable region including an amino acid sequence at least 95%, 96%, 97%, 98%, or 99% identical to the amino acid sequence set forth as amino acids amino acids 1-113 of SEQ ID NO: 8. The antibody specifically binds a polypeptide including a histidine phosphorylated at N3.

In additional embodiments, the antibody includes a heavy chain variable region that includes a) amino acids 1-108 of SEQ ID NO: 1; b) amino acids 1-112 of SEQ ID NO: 2; c) amino acid 1-115 of SEQ ID NO: 3, or d) amino acids 1-115 of SEQ ID NO: 4. In specific non-limiting examples, the heavy chain variable region can be paired with any light chain variable region, provided the antibody specifically binds a polypeptide including a histidine phosphorylated at N3.

In some embodiments, the antibody includes a light chain variable region that includes a) amino acids 1-113 of SEQ ID NO: 5; b) amino acids 1-114 of SEQ ID NO: 6; c) amino acid 1-120 of SEQ ID NO: 7; or d) amino acids 1-113 of SEQ ID NO: 8. In specific non-limiting examples, the light chain variable region can be paired with any heavy chain variable region, provided the antibody specifically binds a polypeptide including a histidine phosphorylated at N3.

Thus, in specific non-limiting examples, the monoclonal antibody includes a) a heavy chain variable region including amino acids 1-108 of SEQ ID NO: 1 and a light chain variable region including amino acids 1-113 of SEQ ID NO: 5; b) a heavy chain variable region including amino acids 1-112 of SEQ ID NO: 2 and a light chain variable region including amino acids 1-114 of SEQ ID NO: 6; c) a heavy chain variable region including amino acids 1-115 of SEQ ID NO: 3 and a light chain variable region including amino acids 1-120 of SEQ ID NO: 7, or d) a heavy chain variable region including amino acids 1-115 of SEQ ID NO: 4 and light chain variable region including amino acids 1-113 of SEQ ID NO: 8.

In some embodiments, an antibody that specifically binds a polypeptide including a histidine phosphorylated at N3, as disclosed herein, includes up to 10 amino acid substitutions (such as up to 1, 2, 3, 4, 5, 6, 7, 8, or up to 9 amino acid substitutions) in the framework regions of the heavy chain of the antibody, or the light chain of the antibody, or the heavy and light chains of the antibody. The antibody specifically binds a polypeptide including a histidine phosphorylated at N3.

In several embodiments, the constant region of the antibody includes one or more amino acid substitutions to optimize half-life of the antibody. The half-life of IgG Abs is in serum regulated by the neonatal Fc receptor (FcRn). Thus, in several embodiments, the antibody includes an amino acid substitution that increases binding to the FcRn. Several such substitutions are known to the person of ordinary skill in the art, such as substitutions at IgG constant regions T250Q and M428L (see, e.g., Hinton et al., J Immunol., 176:346-356, 2006); M428L and N434S (see, e.g., Zalevsky, et al., Nature Biotechnology, 28:157-159, 2010); N434A (see, e.g., Petkova et al., Int. Immunol., 18:1759-1769, 2006); T307A, E380A, and N434A (see, e.g., Petkova et al., Int. Immunol., 18:1759-1769, 2006); and M252Y, S254T, and T256E (see, e.g., Dall'Acqua et al., J. Biol. Chem., 281:23514-23524, 2006). The antibody can also be an immunoadhesin.

One of skill will realize that conservative variants of the antibodies can be produced. Such conservative variants employed in antigen binding fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions) can be made in the V_{H} or the V_{L} regions to increase yield. Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Additionally, to increase binding affinity of the antibody, the V_{L} and V_{H} segments can be randomly mutated, such as within H-CDR3 region or the L-CDR3 region, in a process analogous to the in vivo somatic mutation process responsible for affinity maturation of antibodies during a natural immune response. Thus, in vitro affinity maturation can be accomplished by amplifying V_{H} and V_{L} regions using PCR primers complementary to the H-CDR3 or L-CDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity.

Random mutagenesis of the V_{L} and V_{H} segments could also be used to alter an undesired sequence dependence for any particular mAb for binding to phosphohistidine. H-CDR2 has been shown to be particularly important for recognition of phosphoamino acids by pSer, pThr and pTyr-specific mAbs (Koerber et al., Nature Biotechnology 31:10 2013). Mutagenesis of H-CDR2 can be used to engineer antibodies with improved sequence-independence and affinity for histidine phosphorylated at N3.

Chimeric antibodies are also provided. The antibodies can include any suitable framework region, such as (but not limited to) a human framework region. Human framework regions, and mutations that can be made in a human antibody framework regions, are known in the art (see, for example, in U.S. Pat. No. 5,585,089, which is incorporated herein by reference). Alternatively, a heterologous framework region, such as, but not limited to a mouse framework region, can be included in the heavy or light chain of the antibodies. (See, for example, Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.)

The antibodies or antigen binding fragments disclosed herein can be derivatized or linked to another molecule (such as another peptide or protein). In general, the antibodies or portion thereof is derivatized such that the binding to polypeptides including a histidine phosphorylated at N3 is not affected adversely by the derivatization or labeling. For example, the antibody can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

One type of derivatized antibody is produced by crosslinking two or more antibodies (of the same type or of different types, such as to create bispecific or multispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are available from Pierce Chemical Company, Rockford, Ill. Thus, bivalent and multivalent antibodies can be produced, such as including more than one monoclonal antibody or antigen binding from of antibody that specifically bind polypeptides including a histidine phosphorylated at N3. In some examples, the disclosed antibodies are oligomers of antibodies, such as dimers, trimers, tetramers, pentamers, hexamers, septamers, octomers and so on. In some examples, the antibodies are dimers or pentamers.

The monoclonal antibodies disclosed herein can be of any isotype. The monoclonal antibody can be, for example, an IgM or an IgG antibody, such as IgG₁, IgG₂, IgG₃ or an IgG₄. The class of an antibody that specifically binds a polypeptide including a histidine phosphorylated at N3 can be switched with another (for example, IgG can be switched to IgM), according to well-known procedures. For example, a nucleic acid molecule encoding the V_{L} or V_{H} of a disclosed antibody can be operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that specifically binds a polypeptide including a histidine phosphorylated at N3 that was originally IgG, may be class switched to an IgM. Class switching can be used to convert one IgG subclass to another, such as from IgG₁ to IgG₂, IgG₃, or IgG₄.

Antigen binding fragments of the antibodies that specifically bind to polypeptides including a histidine phosphorylated at N3 are also encompassed by the present disclosure, such as single-domain antibodies (for example, VH domain antibodies), Fab, F(ab′)₂, and Fv. These antigen binding fragments retain the ability to specifically bind polypeptides including a histidine phosphorylated at N3. These fragments include:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, a genetically engineered fragment containing the variable region of the light chain and the variable region of the heavy chain expressed as two chains;

(5) Single chain antibody (such as scFv), a genetically engineered molecule containing the variable region of the light chain, the variable region of the heavy chain, linked by a suitable polypeptide linker as a genetically fused single chain molecule;

(6) A dimer of a single chain antibody (scFV₂), defined as a dimer of a scFV (also known as a “mini-antibody”); and

(7) VH single-domain antibody, an antigen binding fragment consisting of the heavy chain variable domain.

Methods of making these fragments are known in the art (see for example, Harlow and Lane, Antibodies: A Laboratory Manual, Cold Spring Harbor Laboratory, New York, 1988).

In some embodiments, the antigen binding fragments are Fv antibodies, which are typically about 25 kDa and contain a complete antigen-binding site with three CDRs per each heavy chain and each light chain. To produce these antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional examples, the Fv fragments include V_{H} and V_{L} chains connected by a peptide linker. These single-chain antigen binding proteins (scFv) are prepared by constructing a structural gene including DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra). Dimers of a single chain antibody (scFV₂), are also contemplated.

Antigen binding fragments can be prepared by proteolytic hydrolysis of the antibody or by expression in E. coli of DNA encoding the fragment. Antigen binding fragments can be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. Fab′ fragments can also be generated by cloning the two chains into expression vectors with an IgK secretion signal and co-expressing them in 293F cells.

In some cases, antigen binding fragments can be prepared by proteolytic hydrolysis of the antibody or by expression in a host cell (such as E. coli) of DNA encoding the fragment. Antigen binding fragments can be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

### Conjugates

Monoclonal antibodies, or antigen binding fragments thereof, that specifically bind polypeptides including a histidine phosphorylated at N3, can be conjugated to an agent, such as an effector molecule or detectable marker, using any number of means known to those of skill in the art. Both covalent and noncovalent attachment means may be used. Conjugates include, but are not limited to, molecules in which there is a covalent linkage of an effector molecule or a detectable marker to an antibody or antigen binding fragment that specifically binds a polypeptide including a histidine phosphorylated at N3. One of skill in the art will appreciate that various effector molecules and detectable markers can be used, including (but not limited to) radioactive agents such as ¹²⁵I, ³²P, ³H and ³⁵S and other detectable labels, enzymes, target moieties, drugs and ligands, etc.

Effector molecules and detectable markers can be linked to an antibody or antigen binding fragment of interest using any number of means known to those of skill in the art. Both covalent and noncovalent attachment means may be used. The procedure for attaching an effector molecule or detectable marker to an antibody or antigen binding fragment varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule or detectable marker. Alternatively, the antibody or antigen binding fragment is derivatized to expose or attach additional reactive functional groups. The derivatization may involve attachment of any of a number of known linker molecules such as those available from Pierce Chemical Company, Rockford, Ill. The linker can be any molecule used to join the antibody or antigen binding fragment to the effector molecule or detectable marker. The linker is capable of forming covalent bonds to the antibody (or antigen binding fragment) and to the effector molecule or detectable marker. Suitable linkers are well known to those of skill in the art and include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side groups (such as through a disulfide linkage to cysteine) or to the alpha carbon amino and carboxyl groups of the terminal amino acids.

Additionally, in several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference in its entirety.

A monoclonal antibody that specifically binds a polypeptide including a histidine phosphorylated at N3 (or antigen binding fragment thereof) can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, Green fluorescent protein (GFP), Yellow fluorescent protein (YFP).

An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

An antibody or antigen binding fragment may be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitopes recognized by a secondary reporter (such as leucine zipper pair sequences, binding sites for secondary antibodies, metal binding domains, epitope tags).

An antibody or antigen binding fragment can be conjugated with a radiolabeled amino acid. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

Means of detecting such detectable markers are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

An antibody or antigen binding fragment can also be derivatized with a chemical group such as polyethylene glycol (PEG), a methyl or ethyl group, or a carbohydrate group. These groups may be useful to improve the biological characteristics of the antibody or antigen binding fragment, such as to increase serum half-life or to increase tissue binding.

The average number of effector molecule or detectable marker moieties per antibody or antigen binding fragment in a conjugate can range, for example, from 1 to 20 moieties per antibody or antigen binding fragment. For some conjugates, the average number of effector molecule or detectable marker moieties per antibody or antigen binding fragment may be limited by the number of attachment sites on the antibody or antigen binding fragment. For example, where the attachment is a cysteine thiol, an antibody or antigen binding fragment may have only one or several cysteine thiol groups, or may have only one or several sufficiently reactive thiol groups through which a linker may be attached. In certain embodiments, the average number of effector molecule or detectable marker moieties per antibody or antigen binding fragment in a conjugate range from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7. See, for example, U.S. Pat. No. 7,498,298, incorporated by reference herein in its entirety. The average number of effector molecule or detectable marker moieties per antibody or antigen binding fragment in preparations of conjugates may be characterized by conventional means such as mass spectroscopy and, ELISA assay.

The loading (for example, effector molecule/antibody ratio) of an conjugate may be controlled in different ways, for example, by: (i) limiting the molar excess of effector molecule-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number or position of linker-effector molecule attachments (such as thioMab or thioFab prepared as disclosed in W02006/03448, incorporated by reference herein in its entirety.

### Nucleotides, Expression Vectors and Host Cells

Nucleic acids encoding the amino acid sequences of antibodies that specifically bind polypeptides including a histidine phosphorylated at N3 are provided. Nucleic acid molecules encoding these antibodies can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same effector molecule, detectable marker or antibody or antigen binding fragment sequence.

Nucleic acid sequences encoding the antibodies that specifically bind polypeptides including a histidine phosphorylated at N3 can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template.

Exemplary nucleic acids including sequences encoding an antibody that specifically binds a polypeptide including a histidine phosphorylated at N3 (or antigen binding fragment thereof) can be prepared by cloning techniques. Examples of appropriate cloning and sequencing techniques, and instructions sufficient to direct persons of skill through cloning are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill

In one example, an antibody or antigen binding fragment of use is prepared by inserting the cDNA which encodes a variable region from an antibody into a vector which includes the cDNA encoding an effector molecule or detectable marker, such as an enzyme or label. The insertion is made so that the variable region and the effector molecule or detectable marker are read in frame so that one continuous polypeptide is produced. Thus, the encoded polypeptide contains a functional Fv region and a functional effector molecule or detectable marker region. In one embodiment, cDNA encoding an enzyme is ligated to a scFv so that the enzyme is located at the carboxyl terminus of the scFv. In several examples, cDNA encoding a horseradish peroxidase or alkaline phosphatase, or a polypeptide marker of interest is ligated to a scFv so that the enzyme (or polypeptide marker) is located at the amino terminus of the scFv. In another example, the label is located at the amino terminus of the scFv. In a further example, cDNA encoding the protein or polypeptide marker is ligated to a heavy chain variable region of an antibody or antigen binding fragment, so that the enzyme or polypeptide marker is located at the carboxyl terminus of the heavy chain variable region. The heavy chain-variable region can subsequently be ligated to a light chain variable region of the antibody or antigen binding fragment using disulfide bonds. In a yet another example, cDNA encoding an enzyme or a polypeptide marker is ligated to a light chain variable region of an antibody or antigen binding fragment, so that the enzyme or polypeptide marker is located at the carboxyl terminus of the light chain variable region. The light chain-variable region can subsequently be ligated to a heavy chain variable region of the antibody or antigen binding fragment using disulfide bonds.

Once the nucleic acids encoding the conjugate, antibody, or fragment thereof, are isolated and cloned, the protein can be expressed in a recombinantly engineered cell such as bacteria, plant, yeast, insect and mammalian cells using a suitable expression vector. One or more DNA sequences encoding the antibody or fragment thereof can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding the antibody or antigen binding fragment or conjugate thereof, can be operatively linked to expression control sequences. An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The polynucleotide sequences encoding the antibody, or antigen binding fragment or conjugate thereof can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding the antibody, labeled antibody, or antigen binding fragment thereof, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use expression systems such as plasmids and vectors of use in producing proteins in cells including higher eukaryotic cells such as the COS, CHO, HeLa, 293, and myeloma cell lines.

Isolation and purification of recombinantly expressed polypeptide can be carried out by conventional means including preparative chromatography and immunological separations. Once expressed, the conjugate, antibody, or antigen binding fragment thereof, can be purified according to standard procedures of the art, including ammonium sulfate precipitation, affinity columns, column chromatography, and the like (see, generally, R. Scopes, Protein Purification, Springer-Verlag, N. Y., 1982). Substantially pure compositions of at least about 90 to 95% homogeneity are disclosed herein, and 98 to 99% or more homogeneity can be used for pharmaceutical purposes. Once purified, partially or to homogeneity as desired, if to be used therapeutically, the polypeptides should be substantially free of endotoxin.

Methods for expression of single chain antibodies and refolding to an appropriate active form, including single chain antibodies, from bacteria such as E. coli have been described and are well-known and are applicable to the antibodies disclosed herein. See, Buchner et al., Anal. Biochem. 205:263-270, 1992; Pluckthun, Biotechnology 9:545, 1991; Huse et al., Science 246:1275, 1989 and Ward et al., Nature 341:544, 1989, all incorporated by reference herein. Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry, 9: 5015-5021, 1970, incorporated by reference herein, and especially as described by Buchner et al., supra. Renaturation is typically accomplished by dilution (for example, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. An exemplary yield is obtained when these two proteins are mixed in a molar ratio such that a 5 fold molar excess of one protein over the other is not exceeded. Excess oxidized glutathione or other oxidizing low molecular weight compounds can be added to the refolding solution after the redox-shuffling is completed.

In addition to recombinant methods, the antibodies, antigen binding fragments and conjugates thereof can be constructed in whole or in part using standard peptide synthesis. Solid phase synthesis of the polypeptides of less than about 50 amino acids in length can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (such as by the use of the coupling reagent N,N′-dicylohexylcarbodimide) are well known in the art.

### Methods of Detection

Methods are provided for detecting the presence of a polypeptide including a histidine phosphorylated at N3 in a subject. In some embodiments, the methods include contacting a cell from a subject with one or more of the antibodies disclosed herein to form an immune complex. The presence (or absence) of the immune complex is then detected. The presence of the immune complex indicates the presence of a histidine phosphorylated at N3 in the polypeptide. The detection methods can involve in vitro detection of the immune complex. In some embodiments, the detection methods distinguish the presence of histidine phosphorylated at N3 in the polypeptide from histidine phosphorylated at N1. In additional embodiments, the detection method distinguish the presence of a histidine phosphorylated at N3 in the polypeptide from an unphosphorylated polypeptide. In additional embodiments, the methods are used to detect phosphorylated proteins in a signal transduction pathway. In yet other embodiments, the methods can be used to quantitate the amount of a polypeptide including a histidine phosphorylated at N3 in a sample.

Examples of polypeptides including a histidine phosphorylated at N3 are presented in the Tables 1 and 2 shown in Example 13. One or more of these polypeptides including a histidine phosphorylated at N3 can be detected. Any combination of the polypeptides listed in Tables 1 and 2 can be used in the present methods. In some embodiments, any combination of the polypeptides listed in Table 2 can be used in the present methods. In further embodiments, the method detects polypeptides that include only a histidine phosphorylated at N3. In other embodiments, the method detects polypeptides that include a histidine phosphorylated at N3 and a histidine phosphorylated at N1.

In some embodiments, 1, 2, 3, 4, 5, 6, 7, 8, 9, or 10 of the listed polypeptides listed in Table 1 and/or Table 2 can be detected. In other embodiments, 5, 10, 15, 20, 25, 30, 35, 40, 45 or 50 of the polypeptides listed in Table 1 and/or Table 2 can be detected.

In one embodiment, a biological sample is obtained, and the presence of a polypeptide including a histidine phosphorylated at N3 is assessed in vitro. For example, such methods include contacting a biological sample with one or more of the conjugates, antibodies, or antigen binding fragments provided herein that specifically bind polypeptide including a histidine phosphorylated at N3 to form an immune complex. The presence (or absence) of the immune complex is then detected. The presence of the immune complex indicates the presence of the polypeptide including a histidine phosphorylated at N3. For example, an increase in the presence of the immune complex in the sample as compared to formation of the immune complex in a control sample indicates the presence of the polypeptide including a histidine phosphorylated at N3. The amount of the immune complex can be quantitated.

A biological sample can be obtained from a mammalian subject of interest, such as human. The sample can be any sample, including, but not limited to, tissue from biopsies, autopsies and pathology specimens. Biological samples also include sections of tissues, for example, frozen sections taken for histological purposes. In some embodiments, the mammalian subject is treated with a therapeutic agent of interest. The biological sample can also be an extract of cells cultured in vitro. In some embodiments, cells are treated with an agent of interest to determine the effect of the agent on phosphorylation of histidine.

When using a control sample along with the test sample, a complex is detected in both samples and any statistically significant difference in the formation of complexes between the samples is indicative of the presence of polypeptide including histidine phosphorylated at N3 in the test sample.

In some examples of the disclosed methods, the antibody or antigen binding fragment that specifically binds a polypeptide including a histidine phosphorylated at N3 is conjugated to a detectable marker. In additional examples, the methods further include contacting a second antibody that specifically binds the antibody (or antigen binding fragment) that specifically binds a polypeptide including a histidine phosphorylated at N3 for a sufficient amount of time to form an immune complex and detecting this immune complex. In some examples, the second antibody is conjugated to a detectable marker. An increase in the presence of this immune complex in a biological sample compared to the presence of the immune complex in a control sample or other standard detects the presence of a polypeptide including a histidine phosphorylated at N3 in the biological sample.

Suitable detectable markers for the antibody or secondary antibody are described and known to the skilled artisan. For example, various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

The antibodies can be used in immunohistochemical assays. These assays are well known to one of skill in the art (see Harlow & Lane, Antibodies, A Laboratory Manual, Cold Spring Harbor Publications, New York (1988), for a description of immunoassay formats. The assays can be, for example, immunohistochemistry (IHC), immunofluorescence (IF), immunoblotting (IB) and variations thereof including protein or peptide spot blots and slot blots, enzyme linked immunosorbant assay ELISA), radioimmunoassay (RIA), Immune Radioimmunometric Assay (IRMA), Enzyme ImmunoAssay (EIA), and CLIA (Chemioluminescent Immune Assay).

In one embodiment, the antibody or antigen binding fragment that specifically binds to a polypeptide including a histidine phosphorylated at N3 is used to detect one or more phosphorylated polypeptides in a sample from a subject. The antibody or antigen binding fragment can be directly labeled. In some embodiments, a biological sample from a subject is contacted with the antibody or antigen binding fragment and the presence of an immune complex is detected.

In further embodiments, an additional sample is obtained from the subject, such as following treatment with a therapeutic agent. After a sufficient amount of time has elapsed, another sample is obtained. The antibody or antigen binding fragment that specifically binds to a polypeptide including a histidine phosphorylated at N3 is used to detect one or more phosphorylated polypeptides in the second sample. In some embodiments, a biological sample from a subject is contacted with the antibody or antigen binding fragment and the presence of an immune complex is detected. In some examples, an increase in the amount of the immune complex compared to a control, such as in a sample taken prior to the treatment, indicates that the treatment is not effective. In other examples, a decrease in the immune complex compared to a control, such as in a sample taken prior to the treatment, indicates that the treatment is effective.

The antibodies can also be used in screening assays, wherein cells, optionally in a high through-put format, are contacted with one or more agents of interest. After a sufficient amount of time has elapsed, a sample of the cells is obtained. Extracts of the cells can be produced. The antibody or antigen binding fragment that specifically binds to polypeptides including a histidine phosphorylated at N3 is used to detect phosphorylated polypeptides in the sample. An alteration in the binding of the antibody to the sample, as compared to a control sample (such as cells not contacted with the agent) or a standard value, indicates that the agent affects phosphorylation. The assay can be used to identify therapeutic agents. The assay can also be used to identify proteins that are phosphorylated in a signal transduction pathway.

Detection of polypeptides including histidine phosphorylated at N3 can be achieved by immunoassay. In some embodiments, the presence of polypeptides including histidine phosphorylated at N3 is assessed in a sample from a subject of interest, such as, but not limited to, a subject with a tumor. Optionally, the presence of polypeptides including histidine phosphorylated at N3 also is assessed in a control sample. In some embodiments, the amount of polypeptides including histidine phosphorylated at N3 is quantified. The amount of polypeptides including histidine phosphorylated at N3 in the sample from the subject of interest can be compared to levels of the polypeptides including histidine phosphorylated at N3 found in the control. The amount of polypeptides including histidine phosphorylated at N3 in the sample from the subject of interest can be compared to can also be compared to a standard value (such as a non-tumor sample, or a value/range of values expected for such a sample). A significant increase or decrease in an amount can be evaluated using statistical methods known in the art, such as increase or decrease of at least 20%, at least 30%, at least 40%, at least 50%, at least 75%, or at least 95%.

Similarly, assays that utilize two antibodies can be used to detect a protein of interest that is phosphorylated at N3 in a sample. In this embodiment, the presence of polypeptides including histidine phosphorylated at N3 is detected using the methods disclosed above. In some embodiments, the amount of polypeptides including histidine phosphorylated at N3 can be quantified. The presence of a polypeptide of interest is also detected using a second antibody that specifically binds the polypeptide of interest. In some embodiments, the amount of the polypeptide of interest can be quantified. In this manner, the presence of the polypeptide of interest, and whether it is phosphorylated at N3, can be determined. Optionally, the amount of the polypeptide of interest that is phosphorylated at N3, and the amount of the polypeptide of interest that is not phosphorylated at N3 can be determined.

In some embodiments, the amount of polypeptides including histidine phosphorylated at N3, the amount of the specific polypeptide of interest, and/or the amount of the polypeptide of interest that is phosphorylated at N3 is compared to a control. The control can be a control subject, such as a subject not treated with a pharmaceutical agent, or a subject known to be healthy, or to another control (such as a standard value or reference value). A significant increase or decrease can be evaluated using statistical methods known in the art.

In some non-limiting examples, a sandwich ELISA can be used to detect the presence or determine the amount of a protein in a sample. In this method, a solid surface is first coated with an antibody or antigen binding fragment that specifically binds a polypeptide including a histidine phosphorylated at N3, as disclosed herein. The test sample containing proteins (such as, but not limited to, a blood, plasma, serum, or urine sample), is then added and the antigen is allowed to react with the bound antibody. Any unbound antigen is washed away. A known amount of enzyme-labeled protein-specific antibody is then allowed to react with the bound protein. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the quantity of the phosphorylated protein present in the sample tested.

In other non-limiting examples, a solid surface is first coated with an antibody or antigen binding fragment that specifically binds the polypeptide of interest. The test sample containing proteins (such as, but not limited to, a blood, plasma, serum, or urine sample), is then added and the antigen is allowed to react with the bound antibody. Any unbound antigen is washed away. A known amount of enzyme-labeled antibody or antigen binding fragment that specifically binds a polypeptide including a histidine phosphorylated at N3, as disclosed herein, is then allowed to react with the bound protein. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the quantity of the phosphorylated protein present in the sample tested.

In an alternative example, a protein can be assayed in a biological sample by a competition immunoassay utilizing protein standards including a histidine phosphorylated at N3 labeled with a detectable substance and an unlabeled antibody or antigen binding fragment that specifically binds a polypeptide including a histidine phosphorylated at N3. In this assay, the biological sample (such as, but not limited to, a blood, plasma, serum, or urine sample), the labeled protein standards and the antibody that specifically binds the polypeptide including a histidine phosphorylated at N are combined and the amount of labeled protein standard bound to the unlabeled antibody is determined. The amount of phosphorylated including a histidine phosphorylated at N3 in the biological sample is inversely proportional to the amount of labeled standard bound to the antibody.

In yet other embodiments, the antibodies described herein can be used in immunohistochemical assays, such as on histological sections, including, but not limited to, a section of a tumor or a fine needle aspirate of a tumor sample. These assays are well known to one of skill in the art (see Harlow & Lane, Antibodies, A Laboratory Manual, Cold Spring Harbor Publications, New York (1988), for a description of immunoassay formats). In these embodiments, a tissue section or cells from a sample of interest is contacted with a first antibody that specifically binds polypeptides including histidine phosphorylated at N3. In some embodiments, the monoclonal antibody or antigen-binding fragment thereof is labeled. In other embodiments, the monoclonal antibody or antigen-binding fragment thereof is unlabeled. The method can also include contacting the biological sample with a second antibody that specifically binds the first monoclonal antibody, wherein the second antibody is labeled. In additional embodiment, the method includes contact the tissue section or cells with a third antibody or antigen binding fragment thereof that specifically binds a polypeptide of interest. In some embodiments, the third monoclonal antibody or antigen-binding fragment thereof is labeled. In other embodiments, the third monoclonal antibody or antigen-binding fragment thereof is unlabeled. The method can also include contacting the biological sample with a fourth antibody that specifically binds the third monoclonal antibody, wherein the second antibody is labeled.

The method can include contacting an antibody as described herein with a sample to bind the polypeptides including a histidine phosphorylated at N3. The antibody can be either mobilized or immobilized and may be labeled or unlabeled. The process may further comprise releasing the polypeptide, protein, protein fragment, or a portion thereof from the antibody. For example, the antibodies can be coupled to biotin by a hydrazide linkage, and the fusion peptides or proteins including a histidine phosphorylated at N3 can then be separated from peptides or proteins that do not include a histidine phosphorylated at N3 through the use of avidin or streptavidin attached to magnetic beads. When the sample is placed in a magnetic field only the peptides or proteins including the histidine phosphorylated at N3 will bind to the magnetic beads via the linkage between the antibody and the bonds between, for example, the biotin and avidin. The polypeptides attached to the beads can be recovered and the others washed away.

Another affinity technique is immunoprecipitation. The use of immunoprecipitation is known to one skilled in the art. See, for example, Molecular Cloning, A Laboratory Manual, 2d Edition, Maniatis, T. et al. eds. (1989) Cold Spring Harbor Press and Antibodies, A Laboratory Manual, Harlow, E. and Lane, D., eds. (1988) Cold Spring Harbor Press. An example of immunoprecipitation is the use of antibodies coupled to beads. The antibodies coupled to the beads can bind directly to polypeptides including a histidine phosphorylated at N3. A method of attaching antibodies to beads is disclosed and described in U.S. Pat. No. 5,011,912, incorporated herein by reference. For example, antibodies can be coupled to beads using a hydrazide linkage. Such methods are generally described with respect to the use of the FLAG® peptide in Brizzard et al., BioTechniques, Vol. 16, pg. 730 (1994). To accomplish separation using this affinity separation technique, a sample is mixed with beads which are coupled to the antibody or antigen binding fragment. Polypeptides including a histidine phosphorylated at N3 will bind to the antibodies (or antigen binding fragment thereof) coupled to the beads, while polypeptides that do not include a histidine phosphorylated at N3 will not bind. The polypeptides bound to the beads can then be recovered by, for example, centrifugation and elution.

Other methods of detection, identification, isolation, capture, and/or purification of polypeptides are well known in the art, see for example, “Principles and Practice of Immunoassay,” Price and Newman, eds., Stochton Press (1991), Molecular Cloning, A Laboratory Manual, 3rd Edition, Sambrook et al. eds., Cold Spring Harbor Press (2001) and Antibodies, A Laboratory Manual. Harlow, E. and Lane, D., eds. (1988) Cold Spring Harbor Press. Accordingly, methods are provided for detecting, identifying, isolating, capturing or purifying a polypeptide from a sample, wherein the method includes contacting an antibody as disclosed herein with the sample to bind a polypeptide including a histidine phosphorylated at N3. The method also can include releasing the polypeptide, protein, protein fragment, or a portion thereof from the antibody.

In some embodiments, affinity purification can be performed using the antibodies disclosed herein. The disclosed antibodies can be conjugated to resins, such as beads. A sample of interest that includes polypeptides is then passed through the column, such that a polypeptide including a histidine phosphorylated at N3 are bound to the column. The bound polypeptides can then be eluted from the column, and optionally the polypeptides including a histidine phosphorylated at N3 can be quantitated.

Following purification of proteins including a histidine phosphorylated at N3, a detection method can be used to identify these polypeptides. In some embodiments, in order to determine the identity of the eluted polypeptides, mass spectrometry can be performed. Mass spectrometry can also be used to quantify peptides in a biological sample, for example using isotopically labeled peptide standards. The application of mass spectrometric techniques to identify proteins in biological samples is known in the art and is described, for example, in Akhilesh et al., Nature, 405:837-846, 2000; Dutt et al., Curr. Opin. Biotechnol., 11:176-179, 2000; Gygi et al., Curr. Opin. Chem. Biol., 4 (5): 489-94, 2000; Gygi et al., Anal. Chem., 72 (6): 1112-8, 2000; and Anderson et al., Curr. Opin. Biotechnol., 11:408-412, 2000.

Separation of ions according to their m/z ratio can be accomplished with any type of mass analyzer, including quadrupole mass analyzers (Q), time-of-flight (TOF) mass analyzers (for example, linear or reflecting) analyzers, magnetic sector mass analyzers, 3D and linear ion traps (IT), Fourier-transform ion cyclotron resonance (FT-ICR) analyzers, Orbitrap analyzers (like LTQ-Orbitrap LC/MS/MS), and combinations thereof (for example, a quadrupole-time-of-flight analyzer, or Q-TOF analyzer). A triple quadropole instrument can be used such as the Q-trap.

In some embodiments, the mass spectrometric technique is tandem mass spectrometry (MS/MS). Typically, in tandem mass spectrometry a protein product, entering the tandem mass spectrometer is selected and subjected to collision induced dissociation (CID). The spectrum of the resulting fragment ion is recorded in the second stage of the mass spectrometry, as a so-called CID or ETD spectrum. Because the CID or ETD process usually causes fragmentation at peptide bonds and different amino acids for the most part yield peaks of different masses, a CID or ETD spectrum alone often provides enough information to determine the presence of a the protein of interest. Suitable mass spectrometer systems for MS/MS include an ion fragmentor and one, two, or more mass spectrometers, such as those described above. Examples of suitable ion fragmentors include, but are not limited to, collision cells (in which ions are fragmented by causing them to collide with neutral gas molecules), photo dissociation cells (in which ions are fragmented by irradiating them with a beam of photons), and surface dissociation fragmentor (in which ions are fragmented by colliding them with a solid or a liquid surface). Suitable mass spectrometer systems can also include ion reflectors or Negative Electron Transfer Dissociation (NETD) MS, which is run at alkaline pH and therefore can be used for pHis analysis.

Prior to mass spectrometry, the sample or fragments of the sample, for example made by digestion with the trypsin protease, can be subjected to one or more dimensions of chromatographic separation, for example, one or more dimensions of liquid or size exclusion chromatography. Representative examples of chromatographic separation include paper chromatography, thin layer chromatography (TLC), liquid chromatography, column chromatography, high performance liquid chromatography (HPLC), fast protein liquid chromatography (FPLC), ion exchange chromatography, size exclusion chromatography, affinity chromatography, high performance liquid chromatography (HPLC), nano-reverse phase liquid chromatography (nano-RPLC), polyacrylamide gel electrophoresis (PAGE), capillary electrophoresis (CE), reverse phase high performance liquid chromatography (RP-HPLC) or other suitable chromatographic techniques. Thus, in some embodiments, the mass spectrometric technique is directly or indirectly coupled with a one, two or three dimensional liquid chromatography technique, such as column chromatography, high performance liquid chromatography (HPLC or FPLC), reversed phase, ion exchange chromatography, size exclusion chromatography, affinity chromatography (such as protein or peptide affinity chromatography, immunoaffinity chromatography, lectin affinity chromatography, etc.), or one, two or three dimensional polyacrylamide gel electrophoresis (PAGE), or one or two dimensional capillary electrophoresis (CE) to further resolve the biological sample prior to mass spectrometric analysis.

A variety of mass spectrometry methods, including iTRAQ® and MRM, can be used. In some embodiments, quantitative spectroscopic methods, such as SELDI, are used to analyze protein expression in a sample. In one example, surface-enhanced laser desorption-ionization time-of-flight (SELDI-TOF) mass spectrometry is used to detect protein expression, for example by using the PROTEINCHIP™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption. Additional methods are disclosed in the examples section below.

Briefly, one version of SELDI uses a chromatographic surface with a chemistry that selectively captures analytes of interest, such as one or more proteins of interest. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector.

In an additional example, the method may include detection of a protein of interest in a sample using an electrochemical immunoassay method. See, e.g., Yu et al., J. Am. Chem. Soc., 128:11199-11205, 2006; Mani et al., ACS Nano, 3:585-594, 2009; Malhotra et al., Anal. Chem., 82:3118-3123, 2010. In this method, a primary antibody or antigen binding fragment that specifically binds polypeptides including a histidine phosphorylated at N3 is conjugated to terminally carboxylated single-wall carbon nanotubes (SWNT), multi-wall carbon nanotubes (MWCNT), or gold nanoparticles (AuNP), which are attached to a conductive surface. A sample (such as a blood, plasma or serum sample) is contacted with the SWNTs, MWCNTs, or AuNPs, and protein in the sample binds to the primary antibody. A second antibody conjugated directly or indirectly to a redox enzyme (such as horseradish peroxidase (HRP), cytochrome c, myoglobin, or glucose oxidase) binds to the primary antibody or to the protein (for example, in a “sandwich” assay). In some examples, the second antibody is conjugated to the enzyme. In other examples, the second antibody and the enzyme are both conjugated to a support (such as a magnetic bead). Signals are generated by adding enzyme substrate (e.g. hydrogen peroxide if the enzyme is HRP) to the solution bathing the sensor and measuring the current produced by the catalytic reduction.

### Methods of Detecting Tumors

Cancer is the second leading cause of human death next to coronary disease in the United States. Worldwide, millions of people die from cancer every year. In the United States alone, as reported by the American Cancer Society, cancer causes the death of well over a half-million people annually, with over 1.2 million new cases diagnosed per year. While deaths from heart disease have been declining significantly, those resulting from cancer generally are on the rise. Cancer is soon predicted to become the leading cause of death.

Cancer is an abnormal state in which uncontrolled proliferation of one or more cell populations interferes with normal biological functioning. The proliferative changes are usually accompanied by other changes in cellular properties, including reversion to a less differentiated, more developmentally primitive state. The in vitro correlate of cancer is called cellular transformation. Transformed cells generally display several or all of the following properties: spherical morphology, expression of fetal antigens, growth-factor independence, lack of contact inhibition, anchorage-independence, and growth to high density.

Methods are provided herein for evaluating cancer risk, for example for determining the likelihood that a subject, such as an otherwise healthy subject, or a subject suspected or at risk of having a tumor, has a tumor or will likely develop the tumor in the future, or that a tumor will become malignant or metastasize. In particular examples, the method can determine if a subject has or will likely develop the tumor in the future. In further examples, the method can determine the likelihood that a pharmaceutical agent (such as a chemotherapeutic or biologic) is effective for treating a subject.

In some examples, a biological sample obtained from the subject, such as, but not limited to, serum, blood, plasma, urine, purified cells (for example, blood cells, such as white blood cells, B cells, T cells, or mononuclear cells), saliva, a biopsy or tissue sample, such as a sample including cells of a tissue sample obtained from the subject are used to predict the subject's risk. In specific non-limiting examples, the sample includes tumor cells.

In some embodiments, the subject is apparently healthy, such as a subject who does not exhibit symptoms of the tumor. In some examples, a healthy subject is one that if examined by a medical professional, would be characterized as healthy and free of symptoms of the tumor. The methods disclosed herein can be used to screen subjects for future evaluation or treatment for tumors. In other embodiments, the methods determine the likelihood that a subject will develop the tumor, or whether the tumor will metastasize. The methods disclosed herein can be used to confirm a prior clinical suspicion of disease.

The method includes obtaining a sample from a subject that includes polypeptides, and determining whether polypeptides that include a histidine phosphorylated at N3 are present using the antibodies disclosed herein. Any of the methods disclosed above can be used to detect and/or quantify polypeptides including a histidine phosphorylated at N3.

Optionally, the identity of the one or more polypeptides including a histidine phosphorylated at N3, in order to determine the phosphorylation status of one of more specific polypeptides of interest. Methods for determining the identity of polypeptides including a histidine phosphorylated at N3, including, but not limited to, the use of additional antibodies and mass spectrometry are disclosed above. In additional embodiments, the quantity of one of more polypeptides including a histidine phosphorylated at N3 is determined. In further embodiments, method can include determining the phosphorylation profile of one or more polypeptides including a histidine that can be phosphorylated at N3 present in the sample.

Examples of polypeptides including a histidine phosphorylated at N3 are presented in Tables 1 and Table 2 shown in Example 13. One or more of these polypeptides including a histidine phosphorylated at N3 can be detected. In some embodiments, one or more of these polypeptides including a histidine phosphorylated at N3 listed in Table 2 can be detected. In some embodiments, 1, 2, 3, 4, 5, 6, 7, 8, 9, or 10 of the polypeptides listed in Table 1 and/or Table 2 can be detected. In other embodiments, 5, 10, 15, 20, 25, 30, 35, 40, 45 or 50 of the polypeptides listed in Table 1 and/or Table 2 can be detected. Any combination of the polypeptides listed in Table 2 can be used in the present methods.

In some embodiments, the presence of one or more polypeptides that include a histidine phosphorylated at N3, or the amount of one or more proteins phosphorylated at N3, or the phosphorylation profile from the sample, is compared to a control. The control can be the phosphorylation profile of polypeptides including a histidine phosphorylated at N3, or the quantity of one of more specific polypeptides including a histidine phosphorylated at N3 that is present in a control sample. The control sample can be a positive control sample, such as a sample from a subject known to have the tumor, or a negative control sample, such as a sample from a subject known not to have the tumor. In other embodiments, the control can be a reference standard (such as an absolute or relative amount of polypeptides including a histidine phosphorylated at N3 expected if the sample is a tumor sample or if the sample is a normal-non-tumor sample).

In some embodiments, it is determined if a particular protein of interest includes a histidine phosphorylated at N3. The amount of a particular polypeptide including a histidine phosphorylated at N3 can increase or decrease, for example relative to a control. Exemplary proteins include PGAM, and those proteins listed in the Examples section.

Thus, it can be determined if a particular polypeptide including a histidine phosphorylated at N3 is increased or decreased, such as by about 10%, about 20%, about 30%, about 40%, about 50%, about 60%, about 70% about 80%, about 90%, about 95%, about 96%, about 97%, about 98%, about 99% or about 100%, as compared to a control. In other embodiments, detection of an increase or decrease in 1, 2, 3, 4, 5, 6, 7, 8, 9, 10, 15, 20, 25, 30, 35, 40, 45, 50 or more polypeptides including a histidine phosphorylated at N3 indicates that the subject has the tumor. An increase or decrease in the overall amount of polypeptides phosphorylated at N3 can also be detected. Thus, it can be determined if the amount of polypeptides phosphorylated at N3 is increased or decreased, such as by about 10%, about 20%, about 30%, about 40%, about 50%, about 60%, about 70% about 80%, about 90%, about 95%, about 96%, about 97%, about 98%, about 99% or about 100%, as compared to a control.

In more embodiments, the control can be sample from a subject known to have the tumor, a sample from a healthy subject, or a reference standard. The comparison can allows determination of the presence of the tumor, and/or the likelihood that the tumor will metastasize. The method can determine if the tumor is benign or malignant, or it can determine if the tumor is aggressive and likely to metastasize.

The tumor can be any tumor of interest, including, but not limited to, lymphoma, breast cancer, lung cancer and colon cancer. Additional examples are skin, breast, brain, cervical carcinomas, testicular carcinomas, head and neck, lung, mediastinum, gastrointestinal tract, genitourinary system, gynecological system, breast, endocrine system, skin, childhood, unknown primary site or metastatic cancer, a sarcoma of the soft tissue and bone, a mesothelioma, a melanoma, a neoplasm of the central nervous system, a lymphoma, a leukemia, a paraneoplastic syndrome, a peritoneal carcinomastosis. The tumor cells can be from: head and neck tumor, comprising tumors of the nasal cavity, paranasal sinuses, nasopharynx, oral cavity, oropharynx, larynx, hypopharynx, salivary glands and paragangliomas, a cancer of the lung, comprising non-small cell lung cancer, small cell lung cancer, a cancer of the mediastinum, a cancer of the gastrointestinal tract, comprising cancer of the esophagus, stomach, pancreas, liver, biliary tree, small intestine, colon, rectum and anal region, a cancer of the genitourinary system, comprising cancer of the kidney, urethra, bladder, prostate, urethra, penis and testis, a gynecologic cancer, comprising cancer of the cervix, vagina, vulva, uterine body, gestational trophoblastic diseases, ovarian, fallopian tube, peritoneal, a cancer of the breast, a cancer of the endocrine system, comprising a tumor of the thyroid, parathyroid, adrenal cortex, pancreatic endocrine tumors, carcinoid tumor and carcinoid syndrome, multiple endocrine neoplasias, a sarcoma of the soft tissue and bone, a mesothelioma, a cancer of the skin, a melanoma, comprising cutaneous melanomas and intraocular melanomas, a neoplasm of the central nervous system, a cancer of the childhood, comprising retinoblastoma, Wilms' tumor, neurofibromatoses, neuroblastoma, Ewing's sarcoma family of tumors, rhabdomyosarcoma, a lymphoma, comprising non-Hodgkin's lymphomas, cutaneous T-cell lymphomas, primary central nervous system lymphoma, and Hodgkin's disease, a leukemia, comprising acute leukemias, chronic myelogenous and lymphocytic leukemias, plasma cell neoplasms, a cancer of unknown primary site, a peritoneal carcinomastosis, a Kaposi's sarcoma, AIDS-associated lymphomas, AIDS-associated primary central nervous system lymphoma, AIDS-associated Hodgkin's disease and AIDS-associated anogenital cancers, a metastatic cancer to the liver, metastatic cancer to the bone, malignant pleural and pericardial effusions and malignant ascites. In some examples, the tumor is a lymphoma, breast cancer, colon cancer, prostate cancer or lung cancer. The tumor can be benign or malignant.

### Method of Predicting Responsiveness to a Therapeutic Agent

Methods are also provided herein for determining if a cancer in a subject is responsive to an agent, such as a chemotherapeutic agent. The chemotherapeutic agent can be a naturally or non-naturally occurring agent. The chemotherapeutic agent can be a biological molecule (e.g., a therapeutic antibody), a chemical compound, or a combination thereof.

Treatment of the conditions described herein are generally initiated after the development of a condition described herein, or after the initiation of a precursor condition (such as dysplasia or development of a benign tumor). Treatment can be initiated at the early stages of cancer, for instance, can be initiated before a subject manifests symptoms of a condition, such as during a stage I diagnosis or at the time dysplasia is diagnosed. However, treatment can be initiated during any stage of the disease, such as but not limited to stage I, stage II, stage III and stage IV cancers. The treatment can be designed to decrease the severity of the symptoms of one of the conditions, or completely removing the symptoms, or reducing metastasis, tumor volume or number of tumors. Treatment can also include increasing the immune response to the tumor, such as by increasing the humoral response. In one example, there is an increase in antibodies that specifically bind the tumor. In some examples, treatment is administered to try to prevent a benign tumor converting into a malignant or even metastatic lesion. However, in other examples, treatment is administered to any subject diagnosed with cancer.

The treatment can be with naturally occurring chemotherapeutic agents, non-naturally occurring chemotherapeutic agent, or combinations thereof. The chemotherapeutic agent can be a cytokine, a chemokine, or a chemical compound. In one example, for the prevention and treatment of cancer, such as lung cancer, colon cancer or prostate cancer, the treatment can be with a cytokine, including interleukin-2 (IL-2), granulocyte macrophage colony stimulating factor (GM-CSF), or interferon, such as interferon (IFN). In another example, this administration is sequential. In other examples, this administration is simultaneous.

Examples of additional chemotherapeutic agents are alkylating agents, antimetabolites, natural products, or hormones and their antagonists. Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocortical suppressants (such as mitotane and aminoglutethimide). Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone). Examples of the most commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

The treatment can be with a biologic, such as an antibody (e.g., Cetuximab, Gemtuzumab, Ibritumomab tiuxetan, Nivolumab, Panitumumab, Rituximab, Tositumomab or Trastuzumab) cytokine, chemokine, or other biological molecule. In some embodiment, the treatment is a non-naturally occurring monoclonal antibody.

Treatment or treating a tumor includes, but is not limited to, reduction in tumor growth or tumor burden, enhancement of an anti-tumor immune response, induction of apoptosis of tumor cells, inhibition of angiogenesis, enhancement of tumor cell apoptosis, and inhibition of metastases. Administration of an effective amount of a chemotherapeutic agent to a subject may be carried out by any means known in the art including, but not limited to intraperitoneal, intravenous, intramuscular, subcutaneous, transcutaneous, oral, nasopharyngeal or transmucosal absorption. The specific amount or dosage administered in any given case will be adjusted in accordance with the specific cancer being treated, the condition, including the age and weight, of the subject, and other relevant medical factors known to those of skill in the art.

In these embodiments, a sample can be taken from a subject prior to initiation of therapy. After therapy is initiated, an additional sample is taken from the subject. Any of the method disclosed above can be used to determine a change in the amount of the one or more proteins that included a histidine phosphorylated at N3 indicates that the therapy is efficacious. In addition, the subject can be monitored over time to evaluate the continued effectiveness of the therapeutic protocol. The effect of different dosages can also be evaluated, by comparing the expression of markers in a sample from the subject receiving a first dose to the expression of the same markers in a sample from the subject receiving a second (different) dose. The methods can be repeated 1, 2, 3, 4, 5, 6, 7, 8, 9, 10 or more times to determine the lowest dose of a pharmaceutical agent that is effective for treating the subject, and/or the shortest duration of administration that is effective for treating the subject. The methods can also be used over the course of a therapeutic regimen to monitor the efficacy of a pharmaceutical agent for the treatment of the subject.

Examples of polypeptides including a histidine phosphorylated at N3 are presented in Tables 1 and Table 2 shown in Example 13. One or more of these polypeptides including a histidine phosphorylated at N3 can be detected. In some embodiments, one or more of these polypeptides including a histidine phosphorylated at N3 listed in Table 2 can be detected. In some embodiments, 1, 2, 3, 4, 5, 6, 7, 8, 9, or 10 of the polypeptides listed in Table 1 and/or Table 2 can be detected. In other embodiments, 5, 10, 15, 20, 25, 30, 35, 40, 45 or 50 of the polypeptides listed in Table 1 and/or Table 2 can be detected. Any combination of the polypeptides listed in Table 2 can be used in the present methods.

In some embodiments, it is determined if a particular protein of interest includes a histidine phosphorylated at N3. The amount of a particular polypeptide including a histidine phosphorylated at N3 can increase or decrease, for example relative to a control. Thus, it can be determined if a particular polypeptide including a histidine phosphorylated at N3 is increased or decreased, such as by about 10%, about 20%, about 30%, about 40%, about 50%, about 60%, about 70% about 80%, about 90%, about 95%, about 96%, about 97%, about 98%, about 99% or about 100%, as compared to a control. In other embodiments, detection of an increase or decrease in 1, 2, 3, 4, 5, 6, 7, 8, 9, 10, 15, 20, 25, 30, 35, 40, 45, 50 or more polypeptides including a histidine phosphorylated at N3 indicates that the subject has the tumor. In yet other embodiments, an increase or decrease in the total amount of polypeptides phosphorylated at N3 can also be detected. Thus, an increase or a decrease in the total amount of polypeptides phosphorylated at N3, such as by about 10%, about 20%, about 30%, about 40%, about 50%, about 60%, about 70% about 80%, about 90%, about 95%, about 96%, about 97%, about 98%, about 99% or about 100%, as compared to a control, indicates that the subject has the tumor.

The method includes evaluating proteins in a sample from a subject, and detecting proteins that included a histidine phosphorylated at N3 in proteins using the antibodies disclosed herein. In some embodiments, the identity of polypeptides including a histidine phosphorylated at N3 can be determined. In other embodiments, the quantity of one of more polypeptides including a histidine phosphorylated at N3 is determined. The method can include comparing the phosphorylation profile of one or more proteins including a histidine that can be phosphorylated at N3 present in the sample.

In some embodiments, the presence of a particular protein phosphorylated at N3, the amount of one or more proteins phosphorylated at N3, or the phosphorylation profile from the sample is compared to a reference standard, such as the phosphorylation profile of polypeptides including a histidine phosphorylated at N3, or the quantity of one of more specific polypeptides including a histidine phosphorylated at N3, or the profile of proteins phosphorylated at N3, in a control sample. The control sample can be a positive control, such as a sample from a subject known to respond to the chemotherapeutic agent, or a negative control, such as a sample from a subject known not to respond to the chemotherapeutic agent. The control can also be a sample from the subject prior to the administration of the therapeutic agent. The control also can be a reference standard (such as an absolute or relative amount of polypeptides having a histidine phosphorylated at N3 expected if the tumor will respond to the chemotherapeutic agent or if the tumor will not respond to the chemotherapeutic agent).

In some embodiments, comparison of one of more proteins phosphorylated at N3 in the sample from the subject with the tumor to the phosphorylation status of the one or more proteins in samples from subjects known to be sensitive or resistant to the chemotherapeutic agent, or to the reference standard, allows prediction of the responsiveness of the tumor to the chemotherapeutic agent. The prediction may indicate that the tumor will respond completely to the chemotherapeutic agent, or it may predict that the tumor will be only partially responsive or non-responsive (i.e. resistant) to the chemotherapeutic agent.

Once a subject's tumor is predicted to be responsive to a particular chemotherapy, then a treatment plan can be developed incorporating the chemotherapeutic agent and an effective amount of the chemotherapeutic agent(s) can be administered to the subject with the tumor. Those of skill in the art will appreciate that the methods do not guarantee that the subjects will be responsive to the chemotherapeutic agent, but the methods will increase the probability that the selected treatment will be effective to treat the tumor. Also encompassed is the ability to predict the responsiveness of the tumor to multiple chemotherapeutic agents and then to develop a treatment plan using a combination of two or more chemotherapeutic agents.

The disclosed methods can also be used to determine the lowest dose of a chemotherapeutic agent effective to treat a subject. The method includes administering a dose of the chemotherapeutic agent, and detecting polypeptides that included a histidine phosphorylated at N3 in proteins using the antibodies disclosed herein. In some embodiments, the identity of polypeptides including a histidine phosphorylated at N3 can be determined. In other embodiments, the quantity of one of more polypeptides including a histidine phosphorylated at N3 is determined. The method can include comparing the phosphorylation profile of one or more proteins including a histidine that can be phosphorylated at N3 present in a sample from the subject prior to treatment with the dose of chemotherapeutic agent.

In some embodiments, it can be determined if a particular polypeptide including a histidine phosphorylated at N3 is increased or decreased, such as by about 10%, about 20%, about 30%, about 40%, about 50%, about 60%, about 70% about 80%, about 90%, about 95%, about 96%, about 97%, about 98%, about 99% or about 100%, as compared to a control. In other embodiments, an increase or decrease in 1, 2, 3, 4, 5, 6, 7, 8, 9, 10, 15, 20, 25, 30, 35, 40, 45, 50 or more polypeptides including a histidine phosphorylated at N3 can be detected. An increase or decrease in the total amount of polypeptides phosphorylated at N3 can also be detected. Thus, it can be determined if the total amount of polypeptides phosphorylated at N3 is increased or decreased, such as by about 10%, about 20%, about 30%, about 40%, about 50%, about 60%, about 70% about 80%, about 90%, about 95%, about 96%, about 97%, about 98%, about 99% or about 100%. The increase or decrease is measured as compared to the control, such as a sample from the subject prior to treatment with the dose of the chemotherapeutic agent.

The methods can be repeated 1, 2, 3, 4, 5, 6, 7, 8, 9, 10 or more times to determine the lowest dosage of a chemotherapeutic agent that is effective for treating the subject, and/or the shortest duration of administration that is effective for treating the subject. The methods can also be used over the course of a therapeutic regimen to monitor the efficacy of a chemotherapeutic agent for the treatment of the subject.

The tumor can be any tumor of interest, including, but not limited to, lymphoma, breast cancer, lung cancer and colon cancer. The tumor can be benign or malignant. Additional examples are skin, breast, brain, cervical carcinomas, testicular carcinomas, head and neck, lung, mediastinum, gastrointestinal tract, genitourinary system, gynecological system, breast, endocrine system, skin, childhood, unknown primary site or metastatic cancer, a sarcoma of the soft tissue and bone, a mesothelioma, a melanoma, a neoplasm of the central nervous system, a lymphoma, a leukemia, a paraneoplastic syndrome, a peritoneal carcinomastosis. The tumor cells can be from: head and neck tumor, comprising tumors of the nasal cavity, paranasal sinuses, nasopharynx, oral cavity, oropharynx, larynx, hypopharynx, salivary glands and paragangliomas, a cancer of the lung, comprising non-small cell lung cancer, small cell lung cancer, a cancer of the mediastinum, a cancer of the gastrointestinal tract, comprising cancer of the esophagus, stomach, pancreas, liver, biliary tree, small intestine, colon, rectum and anal region, a cancer of the genitourinary system, comprising cancer of the kidney, urethra, bladder, prostate, urethra, penis and testis, a gynecologic cancer, comprising cancer of the cervix, vagina, vulva, uterine body, gestational trophoblastic diseases, ovarian, fallopian tube, peritoneal, a cancer of the breast, a cancer of the endocrine system, comprising a tumor of the thyroid, parathyroid, adrenal cortex, pancreatic endocrine tumors, carcinoid tumor and carcinoid syndrome, multiple endocrine neoplasias, a sarcoma of the soft tissue and bone, a mesothelioma, a cancer of the skin, a melanoma, comprising cutaneous melanomas and intraocular melanomas, a neoplasm of the central nervous system, a cancer of the childhood, comprising retinoblastoma, Wilms' tumor, neurofibromatoses, neuroblastoma, Ewing's sarcoma family of tumors, rhabdomyosarcoma, a lymphoma, comprising non-Hodgkin's lymphomas, cutaneous T-cell lymphomas, primary central nervous system lymphoma, and Hodgkin's disease, a leukemia, comprising acute leukemias, chronic myelogenous and lymphocytic leukemias, plasma cell neoplasms, a cancer of unknown primary site, a peritoneal carcinomastosis, a Kaposi's sarcoma, AIDS-associated lymphomas, AIDS-associated primary central nervous system lymphoma, AIDS-associated Hodgkin's disease and AIDS-associated anogenital cancers, a metastatic cancer to the liver, metastatic cancer to the bone, malignant pleural and pericardial effusions and malignant ascites. In some examples, the tumor is a lymphoma, breast cancer, colon cancer, prostate cancer or lung cancer.

### Method of Identifying Compounds of Use as Antibiotics

Two-component regulatory systems (TCSs) are found in many bacteria, including gram positive and gram negative bacteria and are one of the main signal transduction systems.

A typical TCS has two components, a histidine kinase (HK) and a cognate response regulator (RR). HKs autophosphorylate at conserved histidine residues in response to environmental or metabolic signals. Phosphoryl groups on the histidine residues of HKs are then transferred to conserved aspartate residues in the receiver domains of cognate RRs. Phosphorylation of an RR alters its conformation and its interactions with other components of the signal transduction pathway, which can result in an alteration in the RR to bind to DNA and influence transcription (see Gilmore et al., J. Bacteriol. 187: 8196-8200, 2005). TCSs have been proposed as targets for new antibiotics. The antibodies disclosed herein can be used to evaluate phosphorylation of histidine kinases in bacteria, and thus can be used to determine if an agent of interest increases or decreases phosphorylation. The antibodies disclosed herein can be used to measure phosphorylation of polypeptide that include histidine phosphorylated at N3.

Histidine Kinase and Response Regulator (HK/RR) Pairs that
Regulate Virulence Signaling in Pathogenic Bacteria

| Bacteria | HK | RR |
|---|---|---|
| Pseudomonas aeruginosa | PhoQ | PhoP |
| Salmonela enterica | GacS | GacA |
| Myobacterium tuberculosis | MtrB | MtrA |
| Staphylococcus aureus | WalK | WalR |
| Staphylococcus aureus | ArgC | ArgA |
| Enterococcus faecalis | FsrC | FsrA |
| Bordetella pertussis | BvgS | BvgA |

In some embodiments, cells expressing the two-component regulator system, including a histidine kinase and a cognate response regulator are contacted with an agent of interest, also referred to as a test agent (see, for example, Foster et al., Microbiology 150: 885-896, 2004. The cells can be bacterial cells, such as, but not limited to, E. coli. The amount of the histidine kinase including a histidine phosphorylated at N3, and/or the amount of the cognate response regulator including a histidine phosphorylated at N3 is measured using any of the assays disclosed herein.

The amount of histidine kinase including a histidine phosphorylated at N3, and/or the amount of the cognate response regulator including a histidine phosphorylated at N3 can be compared to a control, such as a reference value, or the amount of histidine kinase including a histidine phosphorylated at N3, and/or the amount of the cognate response regulator including a histidine phosphorylated at N3 present in a cell not contacted with the agent of interest, or contacted with an agent known not to affect a TCS. A decrease in a histidine kinase including a histidine phosphorylated at N3, and/or a decrease in the amount of the activated cognate response regulator including a histidine phosphorylated at N3 indicates that the agent is of use as an antibacterial. In several examples, a decrease in a histidine kinase including a histidine phosphorylated at N3 is assessed. The decrease can be about 10%, about 20%, about 30%, about 40%, about 50%, about 60%, about 70% about 80%, about 90%, about 95%, about 96%, about 97%, about 98%, about 99% or about 100% as compared to a control. In additional embodiments, a decrease in pAsp in the cognate response regulator indicates the agent is of use as an antibacterial. The decrease can be about 10%, about 20%, about 30%, about 40%, about 50%, about 60%, about 70% about 80%, about 90%, about 95%, about 96%, about 97%, about 98%, about 99% or about 100% as compared to a control.

In one embodiment, high throughput screening methods are used that involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., I Amer. Chem. Soc. 116:2661 (1994)), oligocarbamates (Cho et al., Science 261:1303 (1993)), and/or peptidyl phosphonates (Campbell et al., I Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996)), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

The compounds tested as modulators of phosphorylation can be any small organic molecule, or a biological entity, such as a protein, e.g., an antibody or peptide, a sugar, a nucleic acid, e.g., an antisense oligonucleotide or a ribozyme or siRNA, or a lipid. Typically, test compounds will be small organic molecules, peptides, circular peptides, siRNA, antisense molecules, ribozymes, and lipids.

Essentially any chemical compound can be used as a potential modulator, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Any of the assays disclosed herein can be adapted for high throughput screening. In high throughput assays, either soluble or solid state, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using integrated systems.

### Kits

Kits are also provided. The kits will typically include an antibody or antigen binding fragment that specifically binds a polypeptide including a histidine phosphorylated at N3, and/or a conjugate thereof.

More than one of the conjugates or antibodies or antigen binding fragments that specifically bind a polypeptide including a histidine phosphorylated at N3 can be included in the kit. Thus, the kit can include two or more antibodies that specifically bind a polypeptide including a histidine phosphorylated at N3, or a multivalent or bivalent antibody or antigen binding fragment that specifically binds a polypeptide including a histidine phosphorylated at N3 and a conjugate thereof, or a combination thereof, wherein in some examples each antibody is in a separate container forming the kit. In some embodiments, an antigen binding fragment or conjugate including an antigen binding fragment, such as an Fv fragment, is included in the kit. In one example, such as for in vivo uses, the antibody can be a scFv fragment.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed antibodies, antigen binding fragments, or conjugates. In several embodiments the container may have a sterile access port (for example the container may be an intravenous solution bag or a vial having a stopper piercable by a hypodermic injection needle). A label or package insert indicates that the composition is used for treating the particular condition.

The label or package insert typically will further include instructions for use of a disclosed antibodies or fragments thereof, or conjugates thereof, for example, in a detection method. The package insert typically includes instructions customarily included in commercial packages of diagnostic products that contain information about the usage of the antibodies, such as in particular types of assays. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art. Kits may include recombinant proteins for use as a positive control. For example recombinantly expressed and purified PGAM can be included along with 2,3-DPG and instructions for performing in vitro phosphorylation reactions and analysis by a modified SDS-PAGE method that has been optimized for the preservation and detection of a histidine phosphorylated at N3.

In one example, the kit further includes one or more chemotherapeutic agents, for example in a container separate from the antibody.

The following examples are provided to illustrate particular features of certain embodiments, but the scope of the claims should not be limited to those features exemplified.

### EXAMPLES

Phospho-specific, monoclonal antibodies (mAbs) for phosphoester-forming (P—O) amino acids (phosphoserine, phosphothreonine and phosphotyrosine) can be used in the study of protein phosphorylation in cellular signaling. Histidine (His) phosphorylation is well studied in bacterial signal transduction; however, its role in mammalian signaling remains largely unexplored due to the lack of pHis mAbs and lability of the phosphoramidate (P—N) bond. Both nitrogen atoms (N1 and N3) in histidine's imidazole side chain can be phosphorylated to give rise to one of two pHis isomers; 1-phosphohistidine (1-pHis) and 3-phosphohistidine (3-pHis).

Disclosed herein are mAbs that bind specifically to pHis and can distinguish between both pHis isomers. Antibodies were raised in rabbits by immunization with stable pHis analogues incorporated into degenerate peptide libraries. Two novel screening assays were developed based on the isomer specific auto-phosphorylation of NME1 (Nm23-H1/NDPK) and phosphoglycerate mutase (PGAM), which generate either 1-pHis or 3-pHis respectively. These assays, in combination with immunoblotting bacterial and mammalian cell lysates and sequencing mAb IgG variable domains, were used to characterize anti-1-pHis and anti-3-pHis antibodies and select hybridoma clones for establishment of monoclonal cell lines. The sequence independence of these mAbs was determined by peptide dot blot arrays. The pHis mAbs disclosed herein lack sequence specificity (for antigen binding) and do not cross-react with phosphotyrosine or the other pHis isomer. Thus, they can be used for identification and study of pHis substrates in any species using a variety of immunological, proteomic and biological assays.

### Example 1

### Incorporation of Non-Hydrolyzable pHis Analogues into Degenerate Peptide Libraries

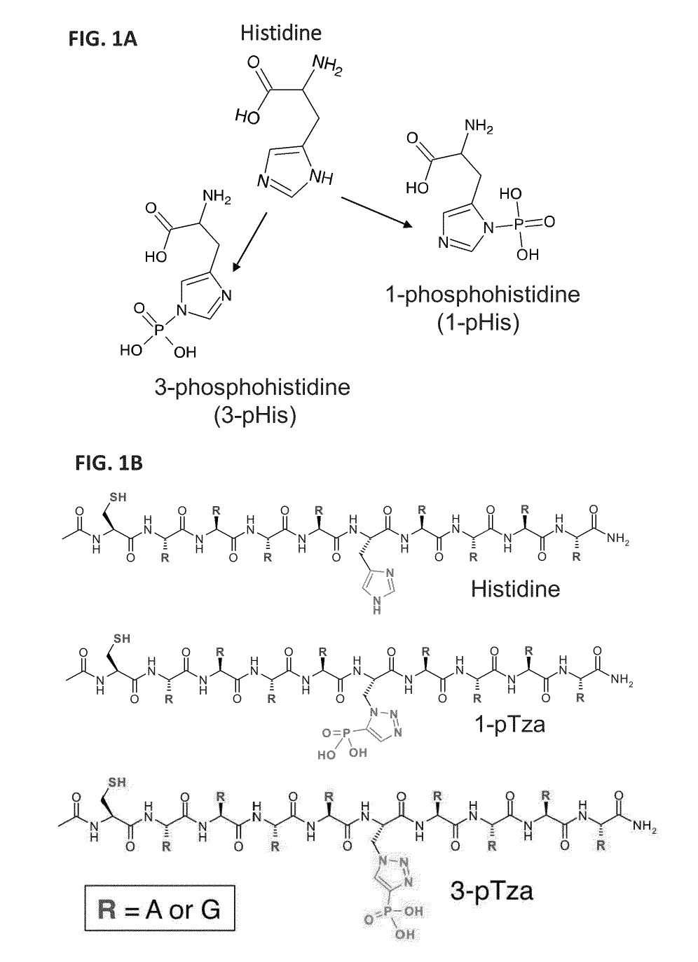

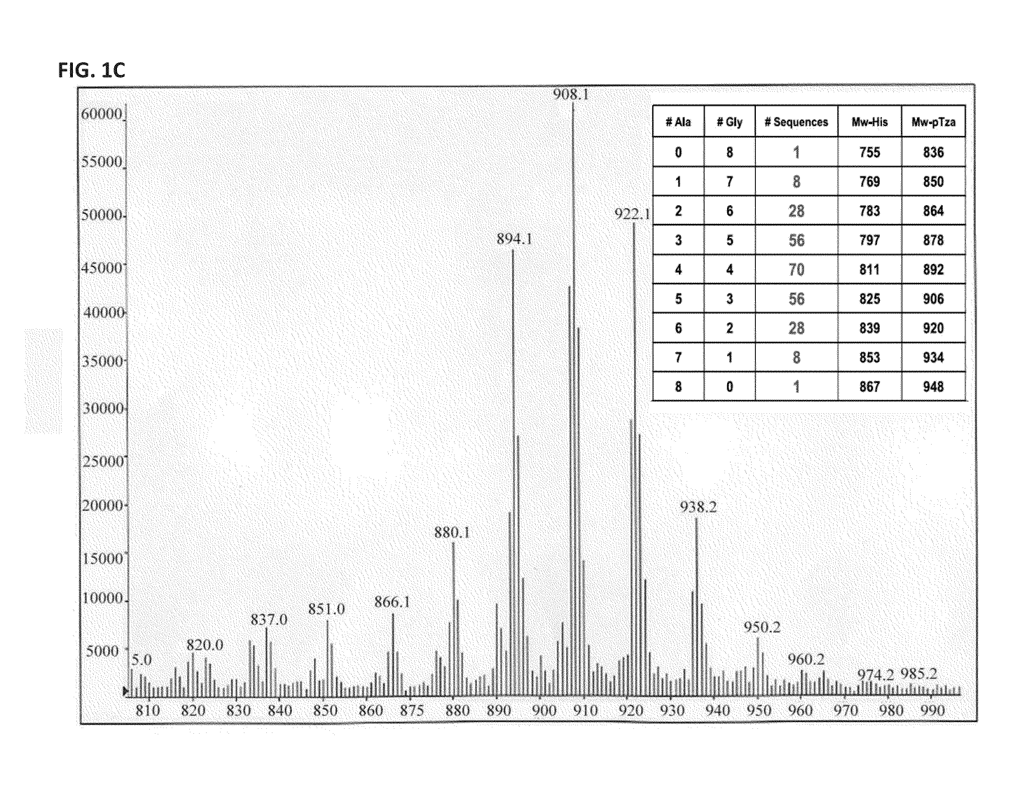

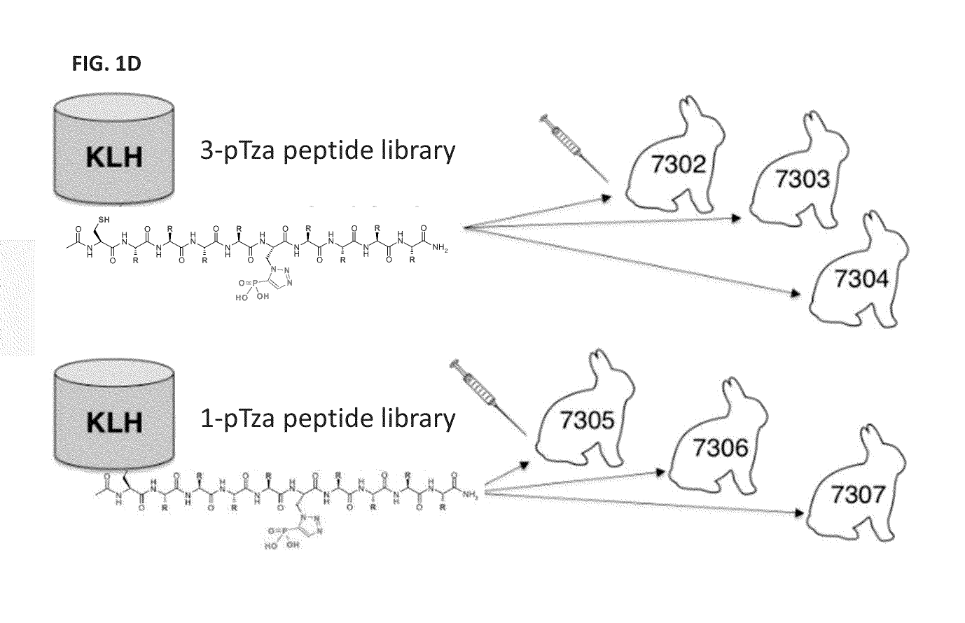

Previous attempts to make pHis antibodies using pHis itself as the antigen have been unsuccessful, presumably because the labile phosphoramidate (P—N) bond is hydrolyzed too rapidly after immunization to elicit an immune response (McAllister et al., Biochemical Society transactions 41, 1072 (August, 2013)). Until recently, the difficulties in creating stable pHis peptides have precluded generation of pHis-specific monoclonal antibodies (mAbs). The development of non-hydrolyzable pHis analogues (Kee et al., Journal of the American Chemical Society, 132, 14327 (October, 2010)) has allowed us to develop a novel strategy for generation of both 1-pHis- and 3-pHis-specific mAbs. Phosphonate (P—C) analogues of both isomers (1-phosphoryltriazolylalanine [1-pTza] and (3-phosphoryltriazolylalanine [3-pTza]) can be synthesized by combining the same starting materials (an azidoalanine derivative and an alkyne) in a click-chemistry reaction using different catalysts. Two peptide libraries were synthesized consisting of 1-pTza or 3-pTza flanked by randomized, neutral, small side chain amino acids (alanine [A] and glycine [G)]) to serve as immunogens to promote generation of sequence-independent anti-pHis antibodies (FIG. 1B). An unphosphorylated version of the peptide libraries (with His in place of the pHis analog (FIG. 1B) was also synthesized as a negative control. MS analysis of the peptide libraries confirmed that incorporation of Ala and Gly occurred randomly and fit with the expected distribution of calculated molecular weights for nine groups of peptides sharing the same composition of 0-8 Ala and/or Gly residues (FIG. 1C). The N-terminal Cys was used to ligate the pTza libraries to the carrier protein Keyhole limpet hemocyanin (KLH) and three rabbits were immunized for each pHis isomer to increase the chance of obtaining antibodies with the desired characteristics (FIG. 1D). Rabbits were chosen for immunization due to recent advances in rabbit hybridoma and monoclonal antibody (RabMAb) technology and unique advantages of the rabbit immune system including; strong immune response to small epitopes, ability to recognize posttranslational modifications with high specificity and pM affinity (Dei Tos et al., Amer. J. Clin. Path. 124, 295 (2005)).

### Example 2

### Generation of 3-pHis Antibodies and Development of PGAM In Vitro Screening Assay

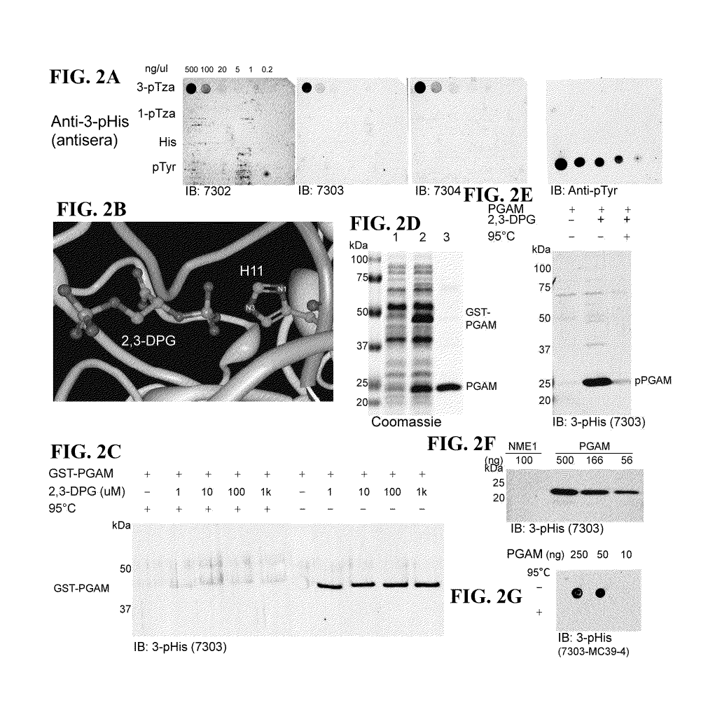

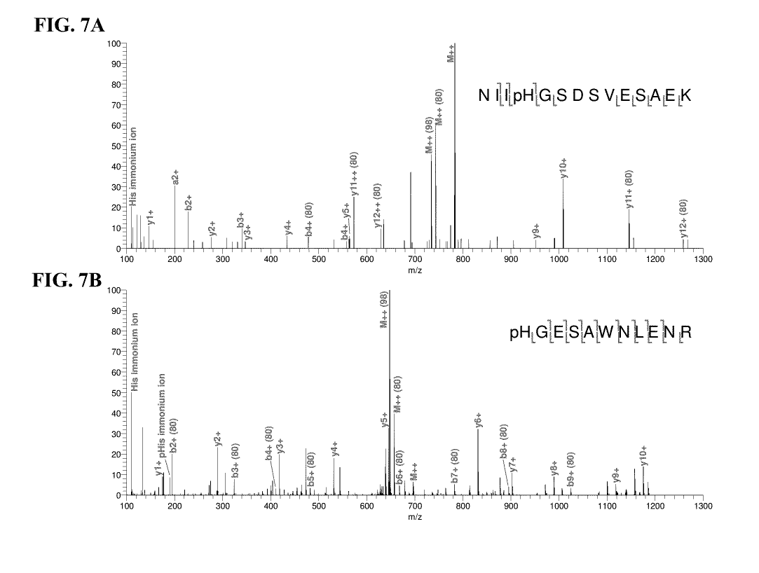

Bleeds from rabbits immunized with 3-pTza peptide library (7302, 7303 and 7304) were screened by dot blot using the immunizing, 3-pTza library (FIG. 2A). The 1-pTza peptide library, the His control library and a pTyr peptide were included as controls. Antisera from all three rabbits detected only the 3-pTza immunizing library and did not cross-react with the other pHis isomer, the His library or a pTyr peptide (FIG. 2A). The data as well as (several) crystal structures (e.g., PDB ID: 1NSP) show that NME1/2 auto-phosphorylation generates only 1-pHis; however, it would be advantageous to use the same protein for screening antibodies to both pHis isomers. It was thus attempted to allow the generation of both pHis isomers on NME1 through mutagenesis of E129. Several mutants were compared with WT NME1 in an in vitro phosphorylation assay. While each mutant tested was still able to autophosphorylate, neither E129L, E129Q nor E129D (E129K did not express in E. coli) elicited any isomer switching suggesting other biochemical, structural or steric factors also favor 1-pHis over 3-pHis. Therefore a different enzyme was needed for generation of 3-pHis and PGAM was chosen due to solved crystal structures of His phosphorylated PGAM and PGAM co-crystallized with its phosphate donor (2,3-diphosphoglycerate [2,3-DPG]) that show the precise location of the phosphate on H11 (PDB ID; 2H4Z and 1E58, FIG. 2B). PGAM converts 3-phosphoglycerate to 2-phosphoglycerate during glycolysis using a unique “ping-pong” mechanism in which a catalytic His residue transfers phosphate by forming a 3-pHis phosphoenzyme intermediate. 2,3-DPG directly phosphorylates only the N3 nitrogen on PGAM (H11)(Vander Heiden et al., Science 329, 1492 (Sep. 17, 2010); Davies et al., Acta Crystallogr 67, 1044 (Sep. 1, 2011); Wang et al., J. Biol. Chem. 281, 39642 (Dec. 22, 2006)). To determine if PGAM could be phosphorylated in vitro, a GST-PGAM fusion (Novus Biological) was incubated with increasing concentrations of 2,3-DPG [1 μm to 1 mM] for 10 min at 30° (FIG. 2C). Reactions were stopped by addition of 5×pH 8.8 sample buffer and immediately analyzed by a modified SDS-PAGE method. Identical samples were heated to 95° C. for 10 min to abolish pHis. Immunoblotting with 3-pHis antisera from rabbit 7303 revealed a heat-sensitive band at the correct size for GST-PGAM (˜45 kDa) that was absent when reactions lacked 2,3-DPG (lane 6). PGAM was subsequently cloned into a bacterial expression vector that allowed cleavage of the GST for analysis of untagged protein. PGAM was purified from E. coli (FIG. 2D) and incubated with or without 2,3-DPG. Immunoblotting with anti-3-pHis antisera revealed a heat-sensitive band at the correct size for un-tagged PGAM (˜25 kDa, FIG. 2E) phosphorylation at the auto-catalytic residue (H11) was confirmed by mass spectrometry (FIG. 7B). As was observed for the 1-pHis antibodies, not all antisera that recognized the pTza analogue could bind actual pHis. In this case, only antisera from 7303 showed a robust signal to 3-pHis on PGAM. At this point, antisera from 7304 did show 3-pHis-specific signal, but it was barely detectable above background. After several subsequent boosts with the 3-pTza peptide library, rabbit 7304 did eventually generate antibodies that were on par with 7303. Splenocytes would eventually be combined from both rabbits to generate hybridomas after an initial attempt with cells from 7303 alone failed to yield any mAbs that could bind 3-pHis, though they could bind the 3-pTza analogue.

To confirm that the anti-3-pHis antibodies did not cross-react with 1-pHis, in vitro phosphorylated NME1 was analyzed along side PGAM (FIG. 2F) and no 1-pHis signal was observed. Identical samples blotted with anti-1-pHis antibodies serve as a positive control for phosphorylation of NME1. To determine 3-pHis antibody sensitivity, in vitro phosphorylated PGAM was diluted 1:5 (250 ng, 50 ng and 10 ng), treated with or without heating and spotted directly on nitrocellulose (FIG. 2G). A multiclonal/monoclonal 3-pHis Ab was able to detect phospho-PGAM. in a heat-sensitive manner.

### Example 3

### Affinity Purification of Polyclonal Anti-1-pHis and Anti-3-pHis Antibodies

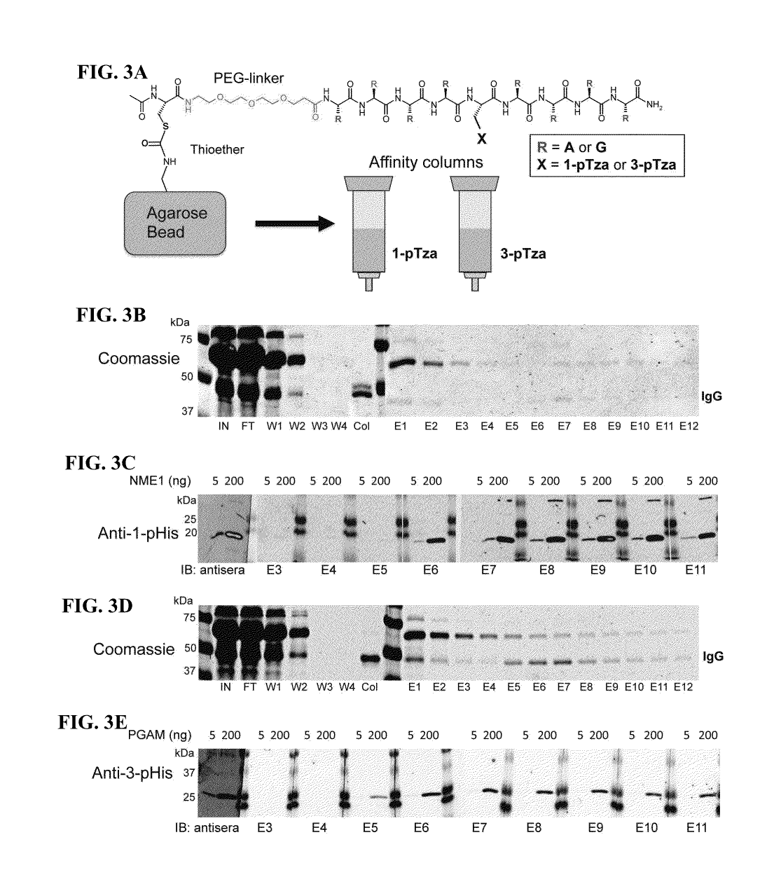

A second version of the 1-pTza and 3-pTza peptide libraries (FIG. 1B) was synthesized with a PEG-linker (polyethylene glycol) inserted between the N-terminal Cys residue and the Ala/Gly/pTza peptide (FIG. 3A). The PEG-linker libraries were immobilized on agarose beads and used to affinity-purify polyclonal pHis antibodies from rabbit antisera. By providing a greater distance between the agarose resin and the pTza analogue, the PEG-linker minimizes steric interference to improve binding of pHis antibodies. Fractions from the purification were analyzed by SDS-PAGE followed by Coomassie staining (FIGS. 3B and 3D) to determine which fractions contained IgG. Elution fractions (E3 to E11) were analyzed by immunoblotting of in vitro phosphorylated NME1 and PGAM for anti-1-pHis (rabbit 7305) and anti-3-pHis (rabbit 7303) antibodies respectively (FIGS. 3C and 3E). Fractions E6 to E11 (and beyond) contained anti-pHis antibodies that could detect as little as 5 ng phospho-NME1 or phospho-PGAM. Identical membranes were probed with crude antisera as a positive control.

### Example 4

### pTza Peptide Dot Blot Screening and Characterization of Anti-1-pHis and Anti-3-pHis Antibodies

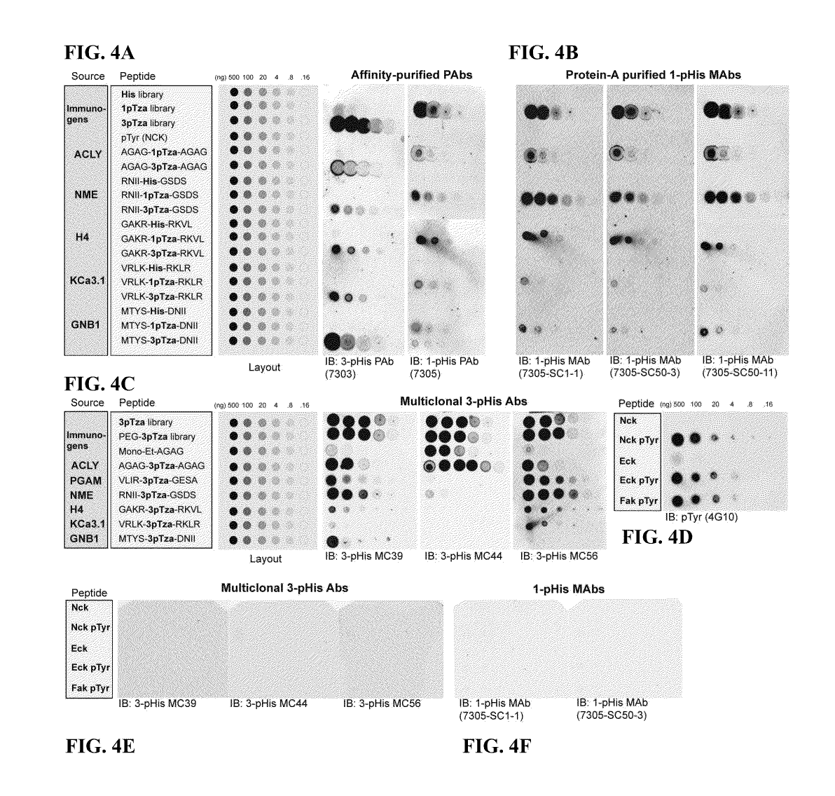

Synthetic peptide dot blot arrays were used to further demonstrate the pHis isoform specificity of the antibodies and determine if they have any amino acid sequence specificity. Peptides of defined sequence were chosen based on the best-characterized mammalian pHis proteins; ACLY, NME1, PGAM, histone H4, KCa3.1 and GNB1. Peptides were synthesized with either His, 1-pTza or 3-pTza flanked by 4 amino acids on either side. Serial dilutions of each peptide (500 ng to 160 pg) and the immunizing pTza and control His peptide libraries were spotted onto nitrocellulose and blotted with affinity-purified, polyclonal anti-1-pHis or 3-pHis antibodies (FIGS. 3C and 3E). The anti-3-pHis antibodies bound only the 3-pTza peptides and the anti-1-pHis antibodies bound only the 1-pTza peptides, regardless of sequence (FIG. 4A). Identical membranes were also probed with several anti-1-pHis mAbs in order to screen and select those with the broadest sequence recognition for future proteomic studies (FIG. 4B). The anti-1-pHis mAbs all showed the same binding profiles and displayed some preference for the NME1/2 H118 peptide. However, with the exception of the KCa3.1 peptide, all 1-pTza peptides were detectable down to 20 ng. As observed previously, ability of an antibody to bind pTza does not always correlate with ability to bind genuine pHis, so further validation and characterization of these mAbs using pHis in proteins is necessary. Since it was shown that the anti-3-pHis antibodies do not cross-react with either 3-pTza or His peptides, peptide arrays including just 3-pTza peptides were provided to determine the sequence specificity of the top 3-pHis mAbs selected from our hybridoma screening efforts (FIG. 4C). A PGAM peptide was included in these arrays since phospho-PGAM was the basis of our screening assay. In contrast to the 1-pHis mAbs tested, the 3-His mAbs displayed some variation in binding profiles. The 3-pHis mAb 7303-MC-39 was able to detect all 3-pTza peptides down to about 800 pg, however binding to the KCa3.1 peptide was relatively poor (˜100 ng). 3-pHis mAb 7304-MC-56 showed similar binding characteristics, however it was better at detecting the KCa3.1 peptide (˜4 ng) while worse at binding the GNB1 peptide. Two or more pHis mAbs could therefore be combined for use as a multiclonal antibody (e.g. the anti-pTyr mAb cocktail 4G10® Platinum, EMD Millipore) for large-scale proteomic identification of pHis substrates in order to broaden the sequence coverage provided by any one pHis mAb. Surprisingly, a highly sequence-specific clone was identified that showed high affinity for the A/G motif peptide based on the sequence of ACLY. 3-pHis mAb 7304-MC44 was able to detect the ACLY peptide and the immunizing peptide library down to ˜160 pg. This indicates that, in this case, the randomized Ala/Gly residues in the degenerate peptide library influenced the sequence specificity of this mAb. Despite its lack of sequence independence, it shows very high sensitivity for this sequence context which is present in at least two important pHis substrates, ACLY and SCS.

### Example 5

### pTyr Peptide Dot Blots and Immunoblotting Reveal No Cross-Reactivity of pHis mAbs

Since some of the early pTyr antibodies were shown to cross-react with pHis (Frackelton, et al., Mol. Cell. Biol. 3, 1343 (Aug. 1, 1983, 1983)) and recently reported “pan-pHis” polyclonal antibodies displayed just a 10-fold higher selectivity for pHis over pTyr (Kee et al., Nat. Chem. Biol. 9, 416 (07//print, 2013)), the pHis mAbs were tested for cross-reactivity using synthetic pTyr peptides. Serial dilutions of three pTyr peptides (based on sequences of Nck, Eck and Fak tyrosine kinases) were spotted on nitrocellulose membranes with along with their un-phosphorylated counterparts. Anti-pTyr antibodies (4G10) detected only the pTyr peptides (FIG. 4D) where as none of the peptides were detected by anti-3-pHis (FIG. 4E) or 1-pHis mAbs (FIG. 4F).

### Example 6

### Screening of Anti-3-pHis Hybridomas

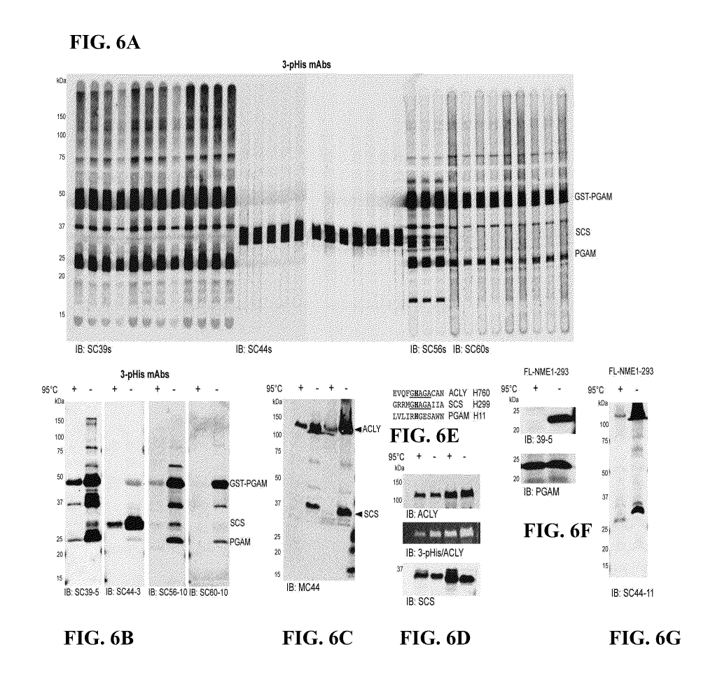

Anti-3-pHis hybridomas were generated from splenocytes harvested from rabbits 7303 and 7304 since antisera from both of these rabbits was able to specifically bind 3-pHis. Hybridomas were subcloned from four parental multi-clonal cell lines (MC39, MC44, MC56 and MC60) selected from 30 ELISA-positive lines. Up to 12 subclones were obtained from each cell line and cell supernatants were initially screened by a 3-pTza peptide ELISA assay and positive clones were selected for secondary screening. Since 3-pHis is involved in bacterial two-component signaling, an E. coli based screening assay was used (FIG. 6A).

E. coli transformed with a pGEX-PGAM plasmid were induced (with IPTG for 3 hr) and crude lysates were loaded on preparative minigels (with a single sample well). After transfer, PVDF membranes were clamped into a slot blotting apparatus to screen up to 40 cell supernatants simultaneously (BioRad Miniprotean II Multiscreen Apparatus). Blotting solutions were normalized to 0.5 ug/ml [IgG] for each cell supernatant and membranes were probed simultaneously with a mouse anti-GST antibody to control for protein loading and GST-NME1 expression and also to assess detection of auto-phosphorylated GST-PGAM by anti-3-pHis mAbs. 30 mM octyl-13-D-glucopyranoside was added to lysis buffer to better solubilize membrane spanning, bacterial histidine kinases. A small-scale screen was performed in parallel using identical E. coli lysates treated with or without heat as a negative control (we had previously shown that phospho-PGAM is sensitive to heat treatment) to determine which bands detected by anti-3-pHis mAbs were 3-pHis specific vs. background or non-specific (FIG. 6B). Identical membranes were probed with crude anti-3-pHis antisera as a positive control and, as expected, the anti-3-pHis mAbs had vastly improved background and non-specific signal levels. Membranes were probed simultaneously with a mouse anti-GST antibody as a loading control.

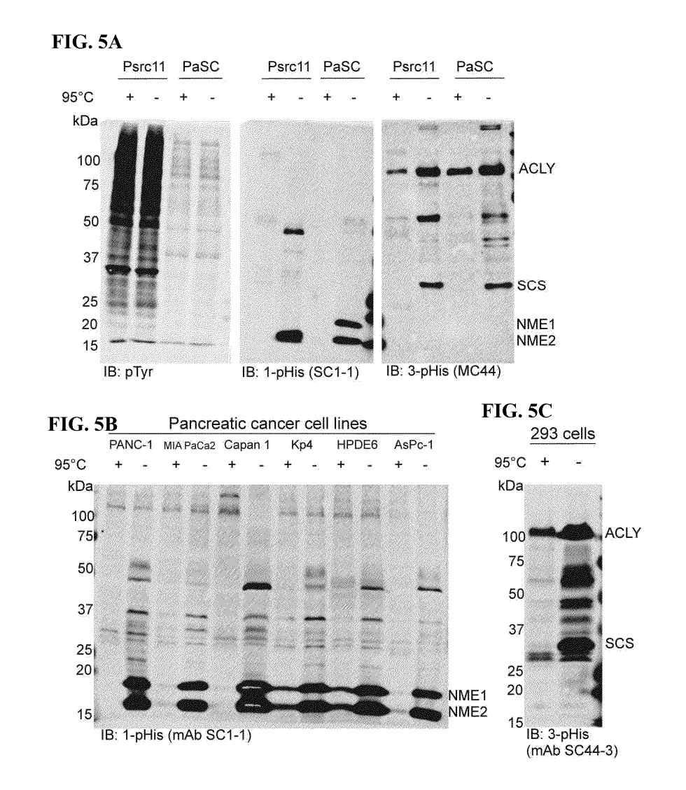

Since the positive control 3-pHis protein, GST-PGAM, is highly overexpressed in these cells, it is not surprising that it represents the strongest signal detected by the anti-3-pHis mAbs, however many other heat-sensitive bands are also detected by the anti-3-pHis mAbs; SC39, SC56 and SC60 indicating they lack sequence specificity (FIGS. 6A and 6B). SC44 only weakly detects PGAM, but strongly detects bacterial SCS (FIG. 6B) and mammalian SCS and ACLY (FIGS. 6C and 6D) which both have the sequence motif; G-H-A-G-A (FIG. 6E)) similar to the ACLY peptide SC44 showed preference for in the pTza peptide dot blots (FIG. 4C). Cell lysates prepared from a stably transfected HEK293 cell line expressing FLAG-Nm23-H1 were blotted with the anti-3-pHis mAb SC39 which detects 3-pHis on endogenously phosphorylated PGAM, but not 1-pHis on FLAG-NME1 (FIG. 6F). These lysates were treated with or without heat to demonstrate that the signal detected by the anti-3-pHis mAbs was heat labile, but the same protein band detected by anti-PGAM antibodies was not. Identical lysates were blotted with the anti-3-pHis mAb SC44 (FIG. 6G) and while many heat-sensitive bands are detected, none of these correspond to patterns of bands detected by anti-1-pHis mAbs (FIG. 5B).

### Example 7

### Immunoblotting of Cell Lysates Using 1-pHis and 3-pHis Antibodies

To test for pTyr cross-reactivity of pHis mAbs on cell lysates, Src-transformed NIH/3T3 fibroblast cell line (Psrc11) were pre-incubated with 1 mM ortho-vanadate for 30 min to enhance pTyr signals. Non-transformed fibroblasts (pancreatic stellate cells PaSC) were tested in parallel as a negative control.

An anti-pTyr mAb (4G10) detected an elevated signal in the psrc11 cells but not in the PaSC negative control, but neither the 1-pHis nor 3-pHis mAbs detected the elevated pTyr signal in Psrc11 cells. Interestingly, many heat-sensitive bands were detected by the anti-pHis mAbs (FIG. 5A). These pHis mAbs also do not cross-react with pSer, pThr, the other pHis isomer or non-phosphorylated His. We used our anti-1-pHis mAbs to immunoblot a number of different pancreatic cancer cell lysates and observed common patterns of heat-sensitive bands (FIG. 5B) indicating many proteins in these cancer cells are similarly regulated by phosphorylation on His. Testing of the 3-pHis mAbs by blotting bacterial and mammalian cell lysates (FIG. 5C) demonstrated that many, heat-sensitive 3-pHis bands were observed.

### Example 8

### Immunofluorescence of pHis Proteins Using Anti-3-pHis mAbs

In addition to validating the anti-3pHis mAbs for immunoblotting, they were tested in other applications, including and immunofluorescence (IF).

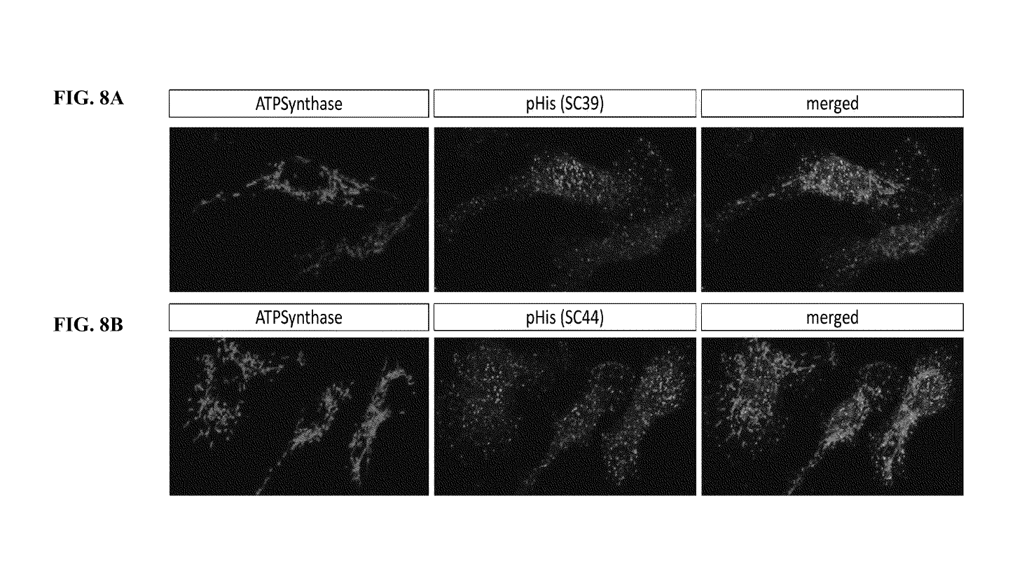

Primary murine macrophages were stained with our anti-3-pHis mAbs (FIGS. 8A and 8B). A staining pattern distinct from that obtained with anti-1-pHis mAb staining was observed, suggesting that different sets of proteins are regulated by 1-pHis and 3-pHis in an isoform-specific manner. In contrast to the anti-1-pHis staining, punctate structures were observed throughout the cytosol, and especially pronounced puncta were visible in the nuclei of these cells. This indicates that some unknown, but specific compartments or organelles have increased 3-pHis signals compared with other regions of the cell. As a negative control, slides were boiled for 10 min in acetic acid and this treatment successfully abolished the observed anti-1-pHis staining.

### Example 9

### Immunoaffinity Chromatography

Anti-1-pHis and anti-3-pHis affinity resins were generated by crosslinking the mAbs to protein-A agarose beads using DSS or BS3 (Pierce). mAbs were coupled to beads at 1 mg IgG/ml of settled protein-A beads. After crosslinking, resins were packed in 10 ml columns (BioRad) and equilibrated with Wash/Binding Buffer (50 mM Tris pH 8.0, 30 mM Sodium carbonate (prepared by dilution of Lysis Buffer with 1 M Tris pH 7.0). Cell lysates were prepared by rinsing with cold TD buffer and scraping cells into 500 ul cold Denaturing Lysis Buffer (Sodium carbonate buffer pH 10.0 [60% Na₂CO₃/40% NaHCO₃] (31), 6 M urea, 1 mM Sodium ortho-vanadate (activated), 30 mM octyl-β-D-glucopyranoside (Sigma, Cat. 08001) supplemented with protease inhibitors (PMSF, pepstatin, leupeptin and aprotinin). Cells were sonicated and clarified (10 min. @ 15,000×g, 4° C.) before diluting lysates 1:5 with Wash/Binding Buffer to decrease urea concentration to 1M prior to binding the affinity column. Lysates were passed over the column 2 times and the column was washed with 4×10 ml Wash/Binding Buffer. pHis proteins/peptides were eluted with 6×500 ul 100 mM triethylamine (TEA), pH 11(Sigma). Elutions were immediately neutralized with 1M ammonium bicarbonate and 20 ul samples were saved from each fraction for SDS-PAGE and western blot analysis. Elution fractions were then lyophilized in order to remove volatile buffer components, store and preserve pHis modified proteins/peptides until LC-MS/MS could be performed.

Immunoblotting of fractions from the 1-pHis mAb and 3-pHis mAb affinity columns was performed to demonstrate that enrichment of pHis proteins was achieved (FIGS. 9 and 10). For mass spectrometry analysis of proteins eluted from the pHis mAb columns (Example 12), elution fractions 1-2 and 3-6 were pooled and analyzed together. Immunoblotting of the elution fractions with anti-pHis mAbs (FIGS. 9A-9B and 10 A-B) shows that the pHis was maintained throughout the immunoaffinity purification. Immunoblotting of elution fractions with protein-specific antibodies including; anti-NME1/2, anti-tubulin and anti-ACLY antibodies shows that these proteins were bound by the 1-pHis mAb column. Subsequent analysis by MS (Example 12) also identified these specific proteins in the pHis mAb column elution fractions.

### Example 10

### Substrates

Primary amino acid sequences of pHis substrates were aligned to look for motifs or commonalities in the residues flanking pHis. Protein names, sequence (SEQ ID NOs. 10-34) and amino acid positions of the pHis residues are shown.

| | | |
|---|---|---|
| 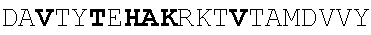 | Histone H4 | H75 |
| 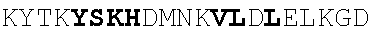 | Annexin I | H246 |
| 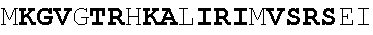 | Annexin I | H293 |
| 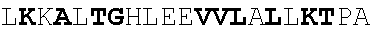 | Annexin I | H103 |
| 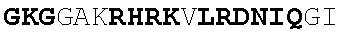 | Histone H4 | H18 |
| 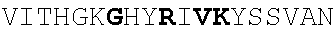 | NSP2 | H225 |
| 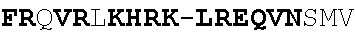 | KCa3.1 | H358 |
| 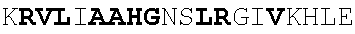 | PGAM 1 | H186 |
| 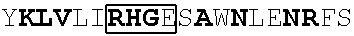 | PGAM 1 | H11 |
| 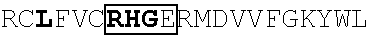 | STS1 (UBASH3B) | H391 |
| KSVLVVRHGERVDQIFGKA | STS2 | |
| 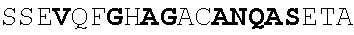 | ACL | H760 |
| 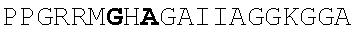 | SCS | H299 |
| 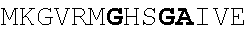 | SCS (yeast) | |
| 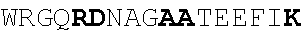 | Aldolase C (rat) | D319 |
| 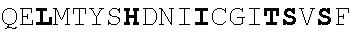 | Gβ₁ GNB1 | H266 |
| 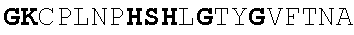 | P-Selectin | H771 |
| 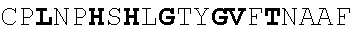 | P-Selectin | H773 |
| 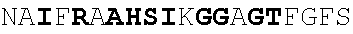 | CheA | H48 |
| 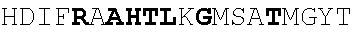 | CHEA_BACSU | H46 |
| 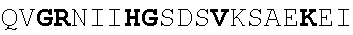 | Nm23-H2 | H118 |
| 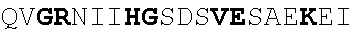 | Nm23-H1 | H118 |
| 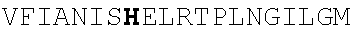 | Sln1 | H576 |
| 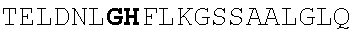 | Ypd1 (HPt) | H64 |
| 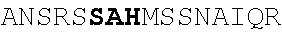 | Cdc10 | H314 |

### Example 11

### Immunofluorescence Staining Reveals Association of 1-pHis with Outer Membrane of Phagosomes

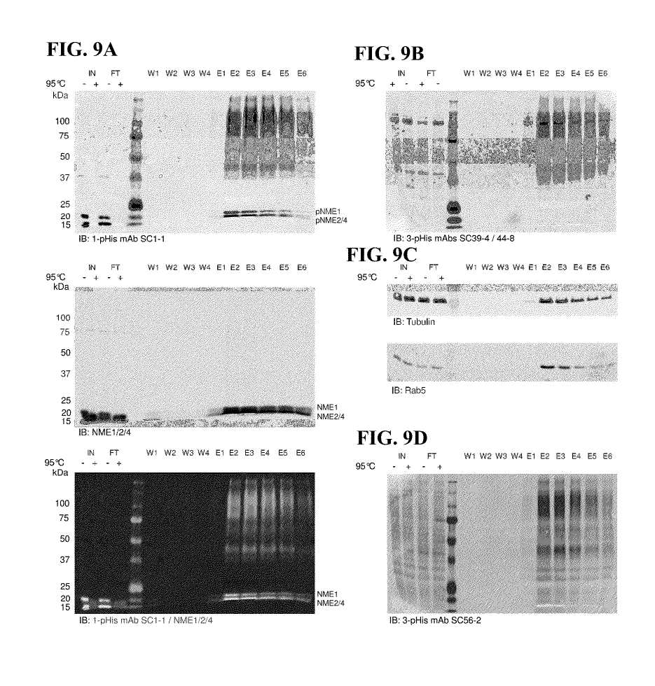

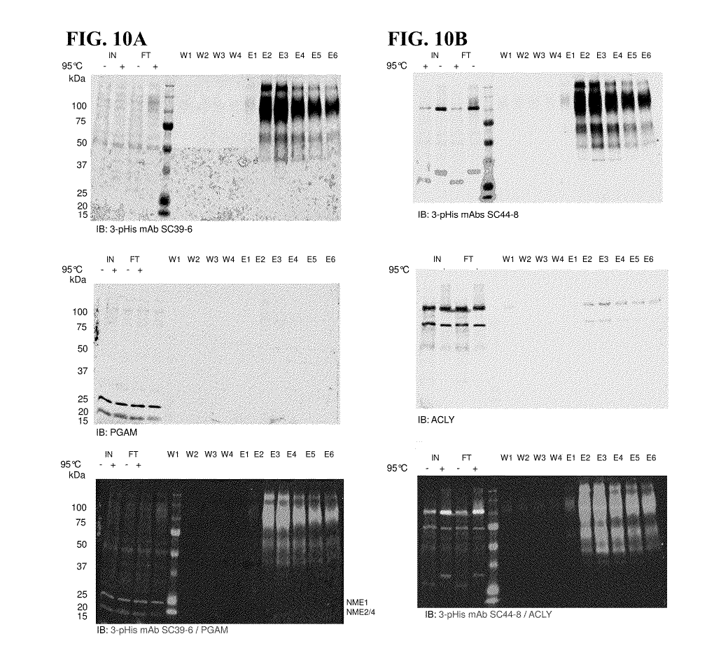

To test the ability of these mAbs to detect pHis proteins by immunofluorescence staining, HeLa cells were stained with the 1-pHis mAb SC1-1. A distinct staining pattern was observed in which most cells had a large (1-2 μm) compartment that stained strongly in the surrounding region but lacked interior pHis staining (FIG. 11A). It was surmised that these might be acidic compartments such as phagosomes or autophagosomes, and this was tested by using primary murine macrophages to look for specific staining of phagosomes. Macrophages isolated from bone marrow were incubated with fluorescently-labeled dextrans to track internalization into phagosomes. Cells were also incubated with LysoTracker prior to fixation to label acidic compartments. 1-pHis staining was absent in nuclei as well as the interior of phagosomes in macrophages co-labeled with the internalized dextrans and LYSOTRACKER®, but staining was pronounced in the regions surrounding these compartments (FIGS. 11B-11D). Remodeling of the actin cytoskeleton supports the extension of pseudopodia at sites of particle engulfment and F-actin assembles around nascent phagosomes. Co-staining with mAb SC1-1 and phalloidin-TRITC revealed a lack of co-localization of 1-pHis with actin filaments (FIG. 11E).

### Example 12

### 3-pHis mAb Immunofluorescence Reveals Staining of Centrosomes, Spindle Poles and Midbodies

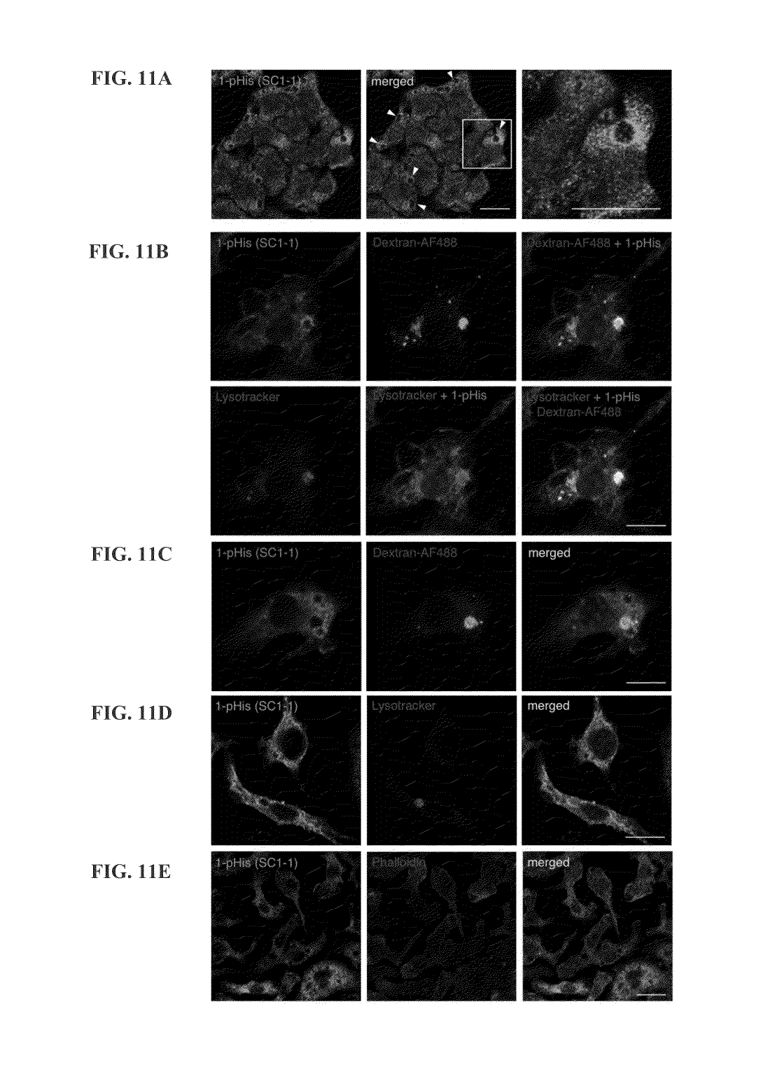

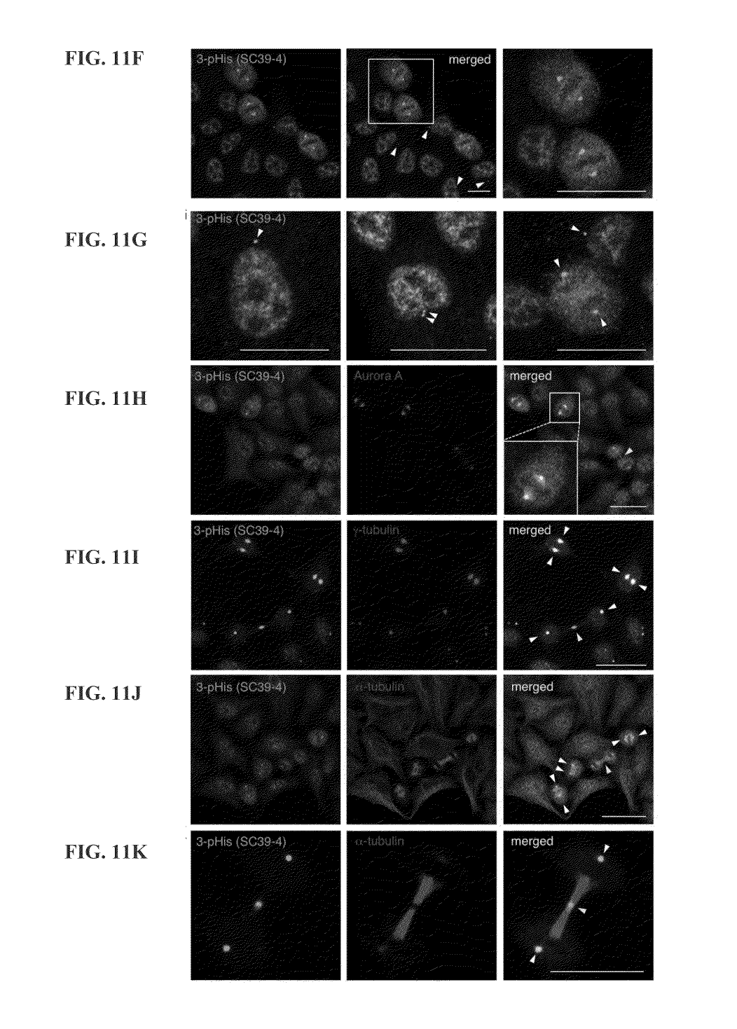

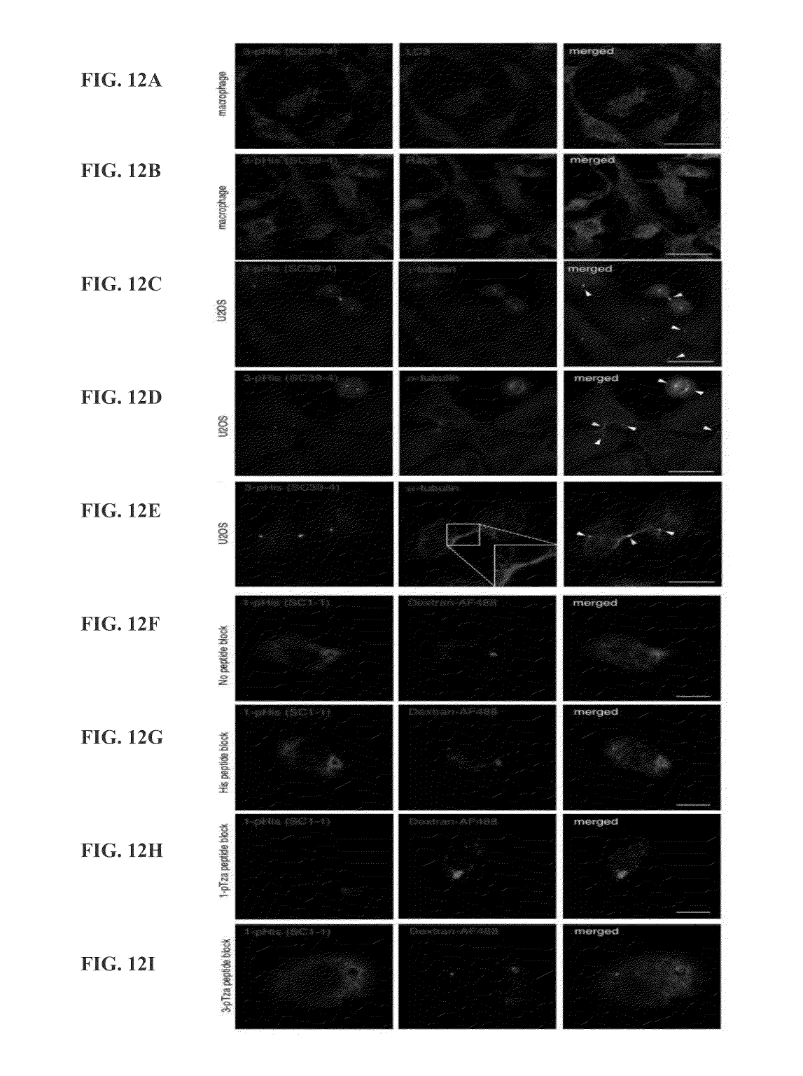

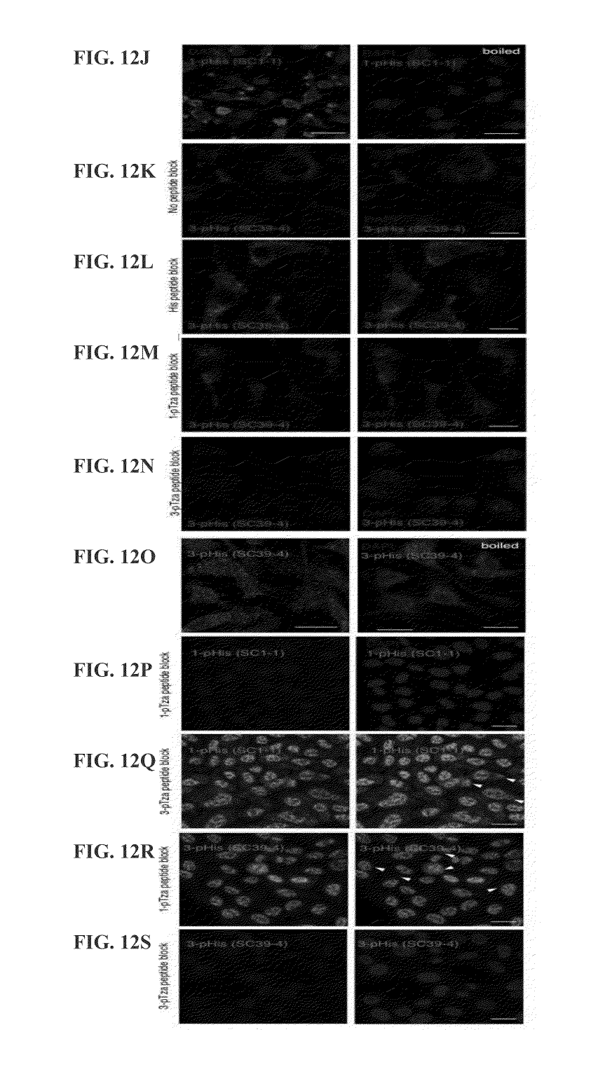

Macrophages stained with 3-pHis mAbs displayed a pattern distinct from 1-pHis staining. Punctate structures were observed throughout the cytosol; however, no co-localization was observed when antibodies specific for organelle markers (e.g., ATP Synthase, LC3, Rab5, α-tubulin and LAMP1, (FIGS. 12A-12B) were tested for co-staining. In contrast to macrophages, staining of HeLa cells with 3-pHis mAbs was primarily nuclear (though curiously absent from nucleoli) and distinctive cell cycle-dependent patterns were observed. Cells in prometaphase through telophase displayed remarkable 3-pHis staining of spindle poles (FIGS. 11F-11K). Interphase cells displayed staining of centrosomes and cells in prophase were observed with duplicated centrosomes (FIG. 11G). An apparent burst of 3-pHis signals was observed in dividing cells and this seemed to last from prometaphase through anaphase. To confirm this observation, HeLa cells were co-stained with 3-pHis mAbs and spindle pole markers Aurora-A and γ-tubulin (FIGS. 12H-12I). To demonstrate that 3-pHis mAbs stained primarily spindle poles and not spindles, cells were co-stained with α-tubulin (FIG. 12J). 3-pHis mAbs also stained structures devoid of Aurora-A, γ-tubulin and α-tubulin in both HeLa and U2Os cells and these appeared to be the midbody of cells in late telophase. (FIGS. 11H-11K and 12C-12E). A series of negative controls using the immunizing pTza peptide libraries were performed. Only the 1-pTza peptides could block 1-pHis staining (FIGS. 12F-12I, 12P-12Q) while only the 3-pTza peptides could block 3-pHis staining (FIGS. 12K-12N, 12R-12S). Additionally, boiling slides for 10 min in citrate buffer reduced both 1-pHis and 3-pHis staining (FIGS. 12J and 12O).

### Example 13

### Enrichment and Identification of Proteins by pHis mAb Immunoaffinity Purification and SILAC LC-MS/NIS

Traditional immunoprecipitation methods are not amenable to pHis preservation and detection. A method for immunoaffinity purification of pHis substrates using immobilized pHis mAbs was developed. Reusable pHis mAb resins were packed in chromatography columns and used to enrich pHis phosphoproteins from cell lysates prior to analysis by LC-MS/MS. pNME1 and pPGAM were used to test the pHis isomer selectivity of the high density mAb columns. NME1 and PGAM were phosphorylated in vitro, denatured (6 M urea, pH 10), mixed together and incubated with either a 1-pHis or 3-pHis mAb column. Purification fractions were immunoblotted with 1- and 3-pHis mAbs as well as NME1 and PGAM antibodies and quantification demonstrates that pNME1 was enriched in elution fractions from the 1-pHis mAb column while pPGAM was enriched in elutions from the 3-pHis mAb column.

In order to determine which proteins bind the columns in a pHis-dependent manner and which are likely false positives that bind non-specifically, stable isotope labeling was used by amino acids in cell culture (SILAC) to metabolically label FLAG-NME1 293 cells. Initially, both the light and heavy labeled (Arg¹³C₆/¹⁵N₄ and Lys ¹³C₆) cells were lysed using identical denaturing and alkaline conditions (6 M urea, pH 10) to preserve pHis. The heavy lysates were then acidified (pH 6) and heated at 65° C. for 30 min to reduce pHis. A dramatic decrease of pHis in the heavy lysates was confirmed by immunoblotting. Both lysates were neutralized, diluted (1:5 to 1 M urea, pH 8), mixed together and one half was passed over the 1-pHis mAb column while the other half was passed over the 3-pHis mAb column. LC-MS/MS analysis was performed on tryptic peptides derived from proteins that eluted off the columns at pH 11. A SILAC ratio (light/heavy) was calculated for each peptide to determine which proteins were enriched in the untreated, light vs. heavy lysates that had been treated to reduce pHis.

Significant enrichment was observed for NME1/2 (55-fold) by the 1-pHis mAb column as well as enrichment of PGAM (4-fold) and other known 3-pHis proteins including; histone H4 (22-fold) and ACLY (11-fold). Proteins corresponding to recently identified pHis phosphopeptides (Lapek et al., 2015, Naunyn-Schmiedeberg's archives of pharmacology 388, 161-173) including; TUBB, TCP1/CCT1, YWHAB, LDHA, RPS3A and GAPDH were also enriched from 5 to 11-fold (Tables 1 and 2).

TABLE 1
SILAC Ratios Determined by LC-MS/MS Analysis
Indicate Enrichment of Known pHis Proteins
by pHis mAb Immunoaffinity Purification

| | | SEQ | | | | | |
|---|---|---|---|---|---|---|---|
| | | ID | | 1pH | 1-pH | 3-pH | 3-pH |
| Protein | Sequence* | NO: | Site | E1 | E2 | E1 | E2 |
| NME1 ^{b} | OVGRNIIHGSDSVES | 35 | H118 | 15.05 | 51.86 | 118 | 2.14 |
| NME2 ^{b} | QVGRNIIHGSDSVKS | 36 | 36H118 | 15.59 | 55.96 | 3.46 | - |
| NME4 | HISRNVIHASDSVEG | 37 | 3751 | - | - | - | - |
| NME5 ^{a} | DDLRNALHGSNDFAA | 38 | H127 | - | - | - | - |
| NME6 ^{a} | TDTRNTTHGSDSVVS | 39 | H129 | - | - | - | - |
| NME7 | DGIRNAAHGPDSFAS | 40 | H206 | - | - | - | - |
| Histone H4 ^{b} | GKGGAKRHRKVLRDN | 41 | H18 | 19.14 | 9.8 | 22.82 | 17.9 |
| KCa3.1 ^{a} | FRQVRLKHRKLREQV | 42 | H358 | - | - | - | - |
| TRPV5 ^{a} | LRQNTLGHLNLGLNL | 43 | H711 | - | - | - | - |
| GNB1 ^{b} | QELMTYSHDNIICGI | 44 | H226 | - | - | - | 1.21 |
| PGAM | YKLVLIRHGESAWNL | 45 | H11 | - | 4.25 | 4.01 | 2.44 |
| ACLY | SSEVQFGHAGACANQ | 46 | H760 | 5.29 | 7.2 | 11.62 | 2.99 |
| SCS | PPGRRMGHAGAIIAG | 47 | H299 | - | 1.68 | - | - |
| P-Selectin ^{a} | GKCPLNPHSHLGTYG | 48 | H771 | - | - | - | - |
| TUBB ^{b} | GNNWAKGHYTEGAEL | 49 | H105 ^{c} | 3.55 | 4.49 | 4.33 | 2.09 |
| TCP1 ^{b} | EETERSLHDAIMIVR | 50 | H346 ^{c} | 6.10 | 5.99 | 5.98 | 3.20 |
| YWHAB ^{b} | KTALCFRHLMKQLLN | 51 | H202 ^{c} | - | 2.04 | 7.43 | 1.63 |
| LDHA ^{b} | GEMMDLQHGSLFLRT | 52 | H67 ^{c} | 4.18 | 6.50 | 6.60 | 3.08 |
| RPS3A | LGKLMELHGEGSSSG | 53 | H232 ^{c} | 7.21 | 9.10 | 10.41 | 4.98 |
| GAPDH | TMEKAGAHLQGGAKR | 54 | H111 ^{c} | 3.40 | 4.45 | 4.76 | 3.32 |

Annotated pHis sites in protein sequences are highlighted in bold.
SILAC ratio (light/heavy) indicates fold enrichment of proteins by pHis mAb affinity purification.
Values from two sequential elution fractions (elution 1 [E1] and elution 2 [E2] are reported as the median value of all quantified peptides that correspond to the listed protein.
^{a} not expressed in cell line tested;
^{b} related protein family members or isoform(s) were enriched;
^{c} sequences from pHis phosphopeptides detected by MS (Lapek et al., 2015, Naunyn-Schmiedeberg's
archives of pharmacology 388, 161-173);
- indicates not detected.
See also Table 2.

In all, 630 proteins (58% of proteins quantified in 1-pHis column elutions E1-E3) and 506 proteins (54% of 3-pHis E1-E3) were enriched by at least 2-fold (Table 2) for a total of 786 different proteins. 280 of these were unique to the 1-pHis column and 156 were unique to the 3-pHis column.

Gene Ontology analysis by biological process (DAVID v6.7 (Huang da et al., 2009, Nature protocols 4, 44-57)) revealed 97 of the 786 genes are involved in cell cycle processes including; PP1, CDK1, cyclin B1, CUL1 and multiple proteasome subunits. Network analysis (STRING v9.1 (Franceschini et al., 2013, Nucleic acids research 41, D808-815)) was performed to visualize the protein-protein interaction network of these cell cycle related genes with known pHis proteins (Table 2).

TABLE 2

| | | 280 | 156 | 786 | 350 |
|---|---|---|---|---|---|
| 630 | 506 | 1-pHis | 3-pHis | 1-pHis | 1-pHis |
| 1-pHis | 3-pHis | only | only | or 3-pHis | and 3-pHis |
| AARS | AAAS | AASDHPPT | AAAS | AAAS | AARS |
| AASDHPPT | AARS | ABCF1 | ABHD10 | AARS | AATF |
| AATF | AATF | ABCF2 | ACTR2 | AASDHPPT | ACAT1 |
| ABCF1 | ABHD10 | ACADM | AK6 | AATF | ACAT2 |
| ABCF2 | ACAT1 | ACADSB | API5 | ABCF1 | ACLY |
| ACADM | ACAT2 | ACO1 | ARFGAP1 | ABCF2 | ACTA1 |
| ACADSB | ACLY | ACO2 | BANF1 | ABHD10 | ACTA2 |
| ACAT1 | ACTA1 | ACTL6A | C1orf131 | ACADM | ACTB |
| ACAT2 | ACTA2 | ACTR1A | C6orf132 | ACADSB | ACTC1 |
| ACLY | ACTB | ADCK3 | CCDC137 | ACAT1 | ACTG1 |
| ACO1 | ACTC1 | AHSA1 | CCNB1 | ACAT2 | ACTG2 |
| ACO2 | ACTG1 | AIMP2 | CELF3 | ACLY | ACTN1 |
| ACTA1 | ACTG2 | AKR1B1 | CENPQ | ACO1 | ACTN4 |
| ACTA2 | ACTN1 | ALDH18A1 | CLASP1 | ACO2 | ACTR3 |
| ACTB | ACTN4 | AP1B1 | CLASP2 | ACTA1 | ADH5 |
| ACTC1 | ACTR2 | AP1G1 | COX5A | ACTA2 | ADSL |
| ACTG1 | ACTR3 | ARF1 | CPNE1 | ACTB | AGK |
| ACTG2 | ADH5 | ARF3 | CPOX | ACTC1 | AHCY |
| ACTL6A | ADSL | ARF5 | CRYZ | ACTG1 | AIFM1 |
| ACTN1 | AGK | ARGLU1 | CSNK2A1 | ACTG2 | ALDH1B1 |
| ACTN4 | AHCY | ARL2 | CSNK2A3 | ACTL6A | ALDH2 |
| ACTR1A | AIFM1 | ARL8A | CYC1 | ACTN1 | ANXA6 |
| ACTR3 | AK6 | ARL8B | CYLD | ACTN4 | APOA1BP |
| ADCK3 | ALDH1B1 | ASNS | DCD | ACTR1A | APRT |
| ADH5 | ALDH2 | ATG3 | DNM1L | ACTR2 | ARCN1 |
| ADSL | ANXA6 | ATIC | DPF2 | ACTR3 | ARF4 |
| AGK | API5 | ATP6V1A | DYNLL2 | ADCK3 | ARPC3 |
| AHCY | APOA1BP | ATP6V1D | EIF3F | ADH5 | ATP5A1 |
| AHSA1 | APRT | BCAP31 | FBL | ADSL | ATP5B |
| AIFM1 | ARCN1 | BCCIP | FHL3 | AGK | ATP5C1 |
| AIMP2 | ARF4 | BUB3 | FSCN1 | AHCY | ATP5F1 |
| AKR1B1 | ARFGAP1 | BYSL | GATAD2A | AHSA1 | ATP5H |
| ALDH18A1 | ARPC3 | BZW2 | GNG12 | AIFM1 | ATP5J |
| ALDH1B1 | ATP5A1 | C11orf48 | GSTM3 | AIMP2 | ATP5J2 |
| ALDH2 | ATP5B | C19orf53 | GSTP1 | AK6 | ATP5J2-PTCD1 |
| ANXA6 | ATP5C1 | C1QBP | H1FX | AKR1B1 | ATP5L |
| AP1B1 | ATP5F1 | CA8 | H2AFJ | ALDH18A1 | ATP5O |
| AP1G1 | ATP5H | CALM1 | HBB | ALDH1B1 | BPNT1 |
| APOA1BP | ATP5J | CALM2 | HBD | ALDH2 | BRIX1 |
| APRT | ATP5J2 | CAND1 | HBE1 | ANXA6 | BSG |
| ARCN1 | ATP5J2-PTCD1 | CAPNS1 | HBG1 | AP1B1 | CA2 |
| ARF1 | ATP5L | CAPZA2 | HBG2 | AP1G1 | CAP1 |
| ARF3 | ATP5O | CBX3 | HIST1H2AB | API5 | CAPZA1 |
| ARF4 | BANF1 | CCAR2 | HIST1H2AC | APOA1BP | CBR1 |
| ARF5 | BPNT1 | CCDC101 | HIST1H2AD | APRT | CCT2 |
| ARGLU1 | BRIX1 | CDC42 | HIST1H2AG | ARCN1 | CCT3 |
| ARL2 | BSG | CHCHD6 | HIST1H2AH | ARF1 | CCT4 |
| ARL8A | C1orf131 | CHORDC1 | HIST1H2AJ | ARF3 | CCT5 |
| ARL8B | C6orf132 | COPB1 | HIST3H2A | ARF4 | CCT6A |
| ARPC3 | CA2 | COPS7A | HMGB1 | ARF5 | CCT7 |
| ASNS | CAP1 | COPS8 | HMGB1P1 | ARFGAP1 | CCT8 |
| ATG3 | CAPZA1 | COX6A1 | HNRNPA1 | ARGLU1 | CDC123 |
| ATIC | CBR1 | COX6A1P2 | HNRNPUL1 | ARL2 | CDC5L |
| ATP5A1 | CCDC137 | CS | HSD17B4 | ARL8A | CDC73 |
| ATP5B | CCNB1 | CTNNA1 | HSP90AA2 | ARL8B | CDK1 |
| ATP5C1 | CCT2 | CUL1 | ITIH2 | ARPC3 | CENPV |
| ATP5F1 | CCT3 | CYB5R3 | KIF15 | ASMS | CLTC |
| ATP5H | CCT4 | DAK | KIF5B | ATG3 | COPA |
| ATP5J | CCT5 | DDB1 | MAPRE1 | ATIC | COPG1 |
| ATP5J2 | CCT6A | DDX18 | MCM2 | ATP5A1 | COX4I1 |
| ATP5J2-PTCD1 | CCT7 | DDX19A | MCM3 | ATP5B | COX5B |
| ATP5L | CCT8 | DDX47 | MRPL11 | ATP5C1 | COX7A2 |
| ATP5O | CDC123 | DDX6 | MRPL41 | ATP5F1 | COX7A2L |
| ATP6V1A | CDC5L | DHX36 | MYH10 | ATP5H | CPNE3 |
| ATP6V1D | CDC73 | DIAPH1 | MYL12A | ATP5J | CSE1L |
| BCAP31 | CDK1 | DIS3 | MYL12B | ATP5J2 | CTPS1 |
| BCCIP | CELF3 | DKC1 | NAA50 | ATP5J2-PTCD1 | CTSD |
| BPNT1 | CENPQ | DLAT | NDUFA12 | ATP5L | DDX1 |
| BRIX1 | CENPV | DNAJB1 | NDUFB4 | ATP5O | DDX17 |
| BSG | CLASP1 | DNAJC7 | NDUFB9 | ATP6V1A | DDX21 |
| BUB3 | CLASP2 | DNM2 | NOL7 | ATP6V1D | DDX39A |
| BYSL | CLTC | DNTTIP2 | NOP56 | BANF1 | DDX39B |
| BZW2 | COPA | DYNC1H1 | NT5DC1 | BCAP31 | DDX3X |
| C11orf48 | COPG1 | ECH1 | NUP93 | BCCIP | DDX46 |
| C19orf53 | COX4I1 | EFTUD2 | OLA1 | BPNT1 | DDX5 |
| C1QBP | COX5A | EIF2S3 | OSBP | BRIX1 | DHX15 |
| CA2 | COX5B | EIF3C | PCDHGA1 | BSG | DHX9 |
| CA8 | COX7A2 | EIF3CL | PCDHGA10 | BUB3 | DLD |
| CALM1 | COX7A2L | EIF3K | PCDHGA11 | BYSL | DNAJA1 |
| CALM2 | CPNE1 | EIF3M | PCDHGA12 | BZW2 | DNAJA2 |
| CAND1 | CPNE3 | EIF4E | PCDHGA2 | C11orf48 | DNAJA3 |
| CAP1 | CPOX | EIF5B | PCDHGA3 | C19orf53 | DRG1 |
| CAPNS1 | CRYZ | EIF6 | PCDHGA4 | C1QBP | DYNLL1 |
| CAPZA1 | CSE1L | ELAVL1 | PCDHGA5 | C1orf131 | DYNLT1 |
| CAPZA2 | CSNK2A1 | EXOSC4 | PCDHGA6 | C6orf132 | EEF1A1 |
| CBR1 | CSNK2A3 | FARSB | PCDHGA7 | CA2 | EEF1A1P5 |
| CBX3 | CTPS1 | FASN | PCDHGA8 | CA8 | EEF1A2 |
| CCAR2 | CTSD | FMR1 | PCDHGA9 | CALM1 | EEF1G |
| CCDC101 | CYC1 | FXR1 | PCDHGB1 | CALM2 | EEF2 |
| CCT2 | CYLD | GEMIN5 | PCDHGB2 | CAND1 | EIF3A |
| CCT3 | DCD | GLO1 | PCDHGB3 | CAP1 | EIF3B |
| CCT4 | DDX1 | GLUD1 | PCDHGB4 | CAPNS1 | EIF3D |
| CCT5 | DDX17 | GNB1 | PCDHGB5 | CAPZA1 | EIF3E |
| CCT6A | DDX21 | GNB2 | PCDHGB6 | CAPZA2 | EIF3I |
| CCT7 | DDX39A | GNL3 | PCDHGB7 | CBR1 | EIF3L |
| CCT8 | DDX39B | GNPNAT1 | PCDHGC3 | CBX3 | EIF4A1 |
| CDC123 | DDX3X | GOT1 | PCDHGC4 | CCAR2 | EIF4A2 |
| CDC42 | DDX46 | GPD2 | PCDHGC5 | CCDC101 | EIF4A3 |
| CDC5L | DDX5 | GPX1 | PDCD10 | CCDC137 | EIF4G1 |
| CDC73 | DHX15 | GTPBP4 | PLEKHJ1 | CCNB1 | EIF4G2 |
| CDK1 | DHX9 | H2AFV | PM20D2 | CCT2 | ERH |
| CENPV | DLD | H2AFZ | PNP | CCT3 | ESD |
| CHCHD6 | DNAJA1 | H3F3A | POLR1C | CCT4 | ETFA |
| CHORDC1 | DNAJA2 | H3F3B | PPIL1 | CCT5 | FAM98A |
| CLTC | DNAJA3 | HDAC1 | PRKDC | CCT6A | FEN1 |
| COPA | DNM1L | HDAC2 | PRPF4B | CCT7 | FHL2 |
| COPB1 | DPF2 | HIST1H3A | PSMAS | CCT8 | FUS |
| COPG1 | DRG1 | HIST2H2AB | PSMA7 | CDC123 | G6PD |
| COPS7A | DYNLL1 | HIST2H3A | PSMC1 | CDC42 | GANAB |
| COPS8 | DYNLL2 | HMGCS1 | PSMC2 | CDC5L | GAPDH |
| COX4I1 | DYNLT1 | HNRNPC | PSMD1 | CDC73 | GARS |
| COX5B | EEF1A1 | HNRNPD | PSMD10 | CDK1 | GART |
| COX6A1 | EEF1A1P5 | HNRNPUL2 | PSMD2 | CELF3 | GDI1 |
| COX6A1P2 | EEF1A2 | HNRNPUL2-BSCL2 | PYCR2 | CENPQ | GDI2 |
| COX7A2 | EEF1G | HPRT1 | RAB11B | CENPV | GMPS |
| COX7A2L | EEF2 | HSPA14 | RAB1A | CHCHD6 | GNB2L1 |
| CPNE3 | EIF3A | IARS | RAB1B | CHORDC1 | GPI |
| CS | EIF3B | IGF2BP1 | RARS | CLASP1 | GTF2I |
| CSE1L | EIF3D | IGF2BP2 | RBM15 | CLASP2 | H2AFY |
| CTNNA1 | EIF3E | IGF2BP3 | RFC4 | CLTC | HADH |
| CTPS1 | EIF3F | IGHG2 | RPL22L1 | COPA | HADHA |
| CTSD | EIF3I | IGHG4 | RPN2 | COPB1 | HADHB |
| CUL1 | EIF3L | IPO5 | RRBP1 | COPG1 | HAT1 |
| CYB5R3 | EIF4A1 | ISOC1 | RRP1B | COPS7A | HIST1H4A |
| DAK | EIF4A2 | ITPA | RRP7A | COPS8 | HIST2H2AA3 |
| DDB1 | EIF4A3 | JUP | SCO1 | COX4I1 | HIST2H2AC |
| DDX1 | EIF4G1 | KARS | SDHB | COX5A | HMGN2 |
| DDX17 | EIF4G2 | KIAA1468 | 11-Sep | COX5B | HNRNPM |
| DDX18 | ERH | KIN27 | SF3B1 | COX6A1 | HNRNPR |
| DDX19A | ESD | KPNA1 | SF3B3 | COX6A1P2 | HNRNPU |
| DDX21 | ETFA | KPNA4 | SFRS2 | COX7A2 | HSD17B10 |
| DDX39A | FAM98A | KTN1 | SFXN1 | COX7A2L | HSP90AA1 |
| DDX39B | FBL | LARS | SLC25A4 | CPNE1 | HSP90AB1 |
| DDX3X | FEN1 | LBR | SLC25A6 | CPNE3 | HSP90B1 |
| DDX46 | FHL2 | LETM1 | SLC3A2 | CPOX | HSPA4 |
| DDX47 | FHL3 | LIG3 | SNRNP35 | CRYZ | HSPH1 |
| DDX5 | FSCN1 | LRPPRC | SNRPD1 | CS | IDH3A |
| DDX6 | FUS | LUC7L2 | SRSF2 | CSE1L | IGHG1 |
| DHX15 | G6PD | MAP2K2 | STOML2 | CSNK2A1 | IGHG3 |
| DHX36 | GANAB | MCM7 | SYNCRIP | CSNK2A3 | ILF2 |
| DHX9 | GAPDH | ME1 | TALDO1 | CTNNA1 | IMMT |
| DIAPH1 | GARS | MMS19 | TBC1D4 | CTPS1 | IQGAP1 |
| DIS3 | GART | MRPL27 | TCEB1 | CTSD | KPNA2 |
| DKC1 | GATAD2A | MRPS11 | TIMM50 | CUL1 | KPNA6 |
| DLAT | GDI1 | MRPS2 | TNN | CYB5R3 | KPNB1 |
| DLD | GDI2 | MRPS33 | TOMM70A | CYC1 | KRBA2 |
| DNAJA1 | GMPS | MRPS9 | TTLL12 | CYLD | KRR1 |
| DNAJA2 | GNB2L1 | MSH6 | UFM1 | DAK | LDHA |
| DNAJA3 | GNG12 | MTR | UPF1 | DCD | LDHB |
| DNAJB1 | GPI | MYBBP1A | UQCR10 | DDB1 | LMNB1 |
| DNAJC7 | GSTM3 | NAA15 | VAT1 | DDX1 | LMNB2 |
| DNM2 | GSTP1 | NACA | VTA1 | DDX17 | LRRC47 |
| DNTTIP2 | GTF2I | NAE1 | VTN | DDX18 | LTA4H |
| DRG1 | H1FX | NAPA | WDR36 | DDX19A | LUC7L |
| DYNC1H1 | H2AFJ | NCAPG | WDR77 | DDX21 | MAGOH |
| DYNLL1 | H2AFY | NDUFAF4 | WDR82 | DDX39A | MAGOHB |
| DYNLT1 | HADH | NDUFB7 | | DDX39B | MARS |
| ECH1 | HADHA | NDUFS1 | | DDX3X | MATR3 |
| EEF1A1 | HADHB | NDUFS3 | | DDX46 | MCM4 |
| EEF1A1P5 | HAT1 | NDUFV1 | | DDX47 | MCM5 |
| EEF1A2 | HBB | NHP2 | | DDX5 | MDC1 |
| EEF1G | HBD | NIPSNAP1 | | DDX6 | MDH1 |
| EEF2 | HBE1 | NOC3L | | DHX15 | MRPL28 |
| EFTUD2 | HBG1 | NOC4L | | DHX36 | MRPS17 |
| EIF2S3 | HBG2 | NUMA1 | | DHX9 | MRPS26 |
| EIF3A | HIST1H2AB | OR5AC2 | | DIAPH1 | MRPS35 |
| EIF3B | HIST1H2AC | PAFAH1B2 | | DIS3 | MT-CO2 |
| EIF3C | HIST1H2AD | PDCD6IP | | DKC1 | MTAP |
| EIF3CL | HIST1H2AG | PDS5A | | DLAT | MTHFD1 |
| EIF3D | HIST1H2AH | PES1 | | DLD | MYH9 |
| EIF3E | HIST1H2AJ | PFAS | | DNAJA1 | NAMPT |
| EIF3I | HIST1H4A | PGD | | DNAJA2 | NAMPTL |
| EIF3K | HIST2H2AA3 | PGM1 | | DNAJA3 | NANS |
| EIF3L | HIST2H2AC | PGM3 | | DNAJB1 | NDUFA10 |
| EIF3M | HIST3H2A | PHF6 | | DNAJC7 | NGDN |
| EIF4A1 | HMGB1 | PLS3 | | DNM1L | NIFK |
| EIF4A2 | HMGB1P1 | POLD3 | | DNM2 | NME1 |
| EIF4A3 | HMGN2 | PPP1CC | | DNTTIP2 | NME1-NME2 |
| EIF4E | HNRNPA1 | PPP2R1A | | DPF2 | NME2 |
| EIF4G1 | HNRNPM | PRDX3 | | DRG1 | NONO |
| EIF4G2 | HNRNPR | PRDX4 | | DYNC1H1 | NOP2 |
| EIF5B | HNRNPU | PREP | | DYNLL1 | NPEPPS |
| EIF6 | HNRNPUL1 | PRKACA | | DYNLL2 | NTPCR |
| ELAVL1 | HSD17B10 | PRMT5 | | DYNLT1 | NUDT5 |
| ERH | HSD17B4 | PRPSAP1 | | ECH1 | OAT |
| ESD | HSP90AA1 | PSMA4 | | EEF1A1 | PA2G4 |
| ETFA | HSP90AA2 | PSMA8 | | EEF1A1P5 | PAICS |
| EXOSC4 | HSP90AB1 | PSMB4 | | EEF1A2 | PARP1 |
| FAM98A | HSP90B1 | PSMD12 | | EEF1G | PCBP1 |
| FARSB | HSPA4 | PSMD13 | | EEF2 | PCBP2 |
| FASN | HSPH1 | PSMD6 | | EFTUD2 | PCNA |
| FEN1 | IDH3A | PSME3 | | EIF2S3 | PDHA1 |
| FHL2 | IGHG1 | PSMG1 | | EIF3A | PDHB |
| FMR1 | IGHG3 | PTBP1 | | EIF3B | PGAM1 |
| FUS | ILF2 | PUS1 | | EIF3C | PGK1 |
| FXR1 | IMMT | PWP2 | | EIF3CL | PHB |
| G6PD | IQGAP1 | PYGL | | EIF3D | PHB2 |
| GANAB | ITIH2 | QARS | | EIF3E | PHGDH |
| GAPDH | KIF15 | RAB5B | | EIF3F | PKM |
| GARS | KIF5B | RAB8A | | EIF3I | POTEF |
| GART | KPNA2 | RAC1 | | EIF3K | PPAT |
| GDI1 | KPNA6 | RALB | | EIF3L | PPP1CA |
| GDI2 | KPNB1 | RALY | | EIF3M | PPP1CB |
| GEMIN5 | KRBA2 | RAP1A | | EIF4A1 | PPP1R7 |
| GLO1 | KRR1 | RBBP4 | | EIF4A2 | PRDX1 |
| GLUD1 | LDHA | RBBP7 | | EIF4A3 | PRDX2 |
| GMPS | LDHB | RBM42 | | EIF4E | PRDX6 |
| GNB1 | LMNB1 | RCC1 | | EIF4G1 | PRIM2 |
| GNB2 | LMNB2 | RCC2 | | EIF4G2 | PRKAG1 |
| GNB2L1 | LRRC47 | RFC5 | | EIF5B | PRMT1 |
| GNL3 | LTA4H | RHOA | | EIF6 | PRPF19 |
| GNPNAT1 | LUC7L | RNH1 | | ELAVL1 | PRPF3 |
| GOT1 | MAGOH | RPA2 | | ERH | PRPS1 |
| GPD2 | MAGOHB | RPL10 | | ESD | PRPSAP2 |
| GPI | MAPRE1 | RPL10A | | ETFA | PSMA2 |
| GPX1 | MARS | RPL13A | | EXOSC4 | PSMC4 |
| GTF2I | MATR3 | RPL14 | | FAM98A | PSMC5 |
| GTPBP4 | MCM2 | RPL21 | | FARSB | PSMC6 |
| H2AFV | MCM3 | RPL22 | | FASN | PSMD11 |
| H2AFY | MCM4 | RPL28 | | FBL | PSMD14 |
| H2AFZ | MCM5 | RPL35A | | FEN1 | PSMD3 |
| H3F3A | MDC1 | RPL37A | | FHL2 | PSMD7 |
| H3F3B | MDH1 | RPN1 | | FHL3 | PTGES2 |
| HADH | MRPL11 | RPS26 | | FMR1 | PTRH2 |
| HADHA | MRPL28 | RPS27A | | FSCN1 | PUF60 |
| HADHB | MRPL41 | RPS28 | | FUS | PURA |
| HAT1 | MRPS17 | RTCB | | FXR1 | PYCR1 |
| HDAC1 | MRPS26 | RUVBL1 | | G6PD | RAB10 |
| HDAC2 | MRPS35 | SAP30 | | GANAB | RAB5C |
| HIST1H3A | MT-CO2 | SAR1A | | GAPDH | RAN |
| HIST1H4A | MTAP | SAR1B | | GARS | RANGAP1 |
| HIST2H2AA3 | MTHFD1 | SCFD1 | | GART | RAP1B |
| HIST2H2AB | MYH10 | SEPT7 | | GATAD2A | RAVER1 |
| HIST2H2AC | MYH9 | SEPT9 | | GDI1 | RBM14 |
| HIST2H3A | MYL12A | SF3A2 | | GDI2 | RBM28 |
| HMGCS1 | MYL12B | SF3B2 | | GEMIN5 | RBM34 |
| HMGN2 | NAA50 | SFRS3 | | GLO1 | RBM39 |
| HNRNPC | NAMPT | SH3GLB1 | | GLUD1 | RPA1 |
| HNRNPD | NAMPTL | SHMT2 | | GMPS | RPL11 |
| HNRNPM | NANS | SLC25A24 | | GNB1 | RPL12 |
| HNRNPR | NDUFA10 | SLIRP | | GNB2 | RPL13 |
| HNRNPU | NDUFA12 | SLTM | | GNB2L1 | RPL15 |
| HNRNPUL2 | NDUFB4 | SMC1A | | GNG12 | RPL18 |
| HNRNPUL2-BSCL2 | NDUFB9 | SMC3 | | GNL3 | RPL18A |
| HPRT1 | NGDN | SMC4 | | GNPNAT1 | RPL19 |
| HSD17B10 | NIFK | SNRNP200 | | GOT1 | RPL23 |
| HSP90AA1 | NME1 | SNRPB | | GPD2 | RPL26 |
| HSP90AB1 | NME1-NME2 | SNRPN | | GPI | RPL26L1 |
| HSP90B1 | NME2 | SNX15 | | GPX1 | RPL27 |
| HSPA14 | NOL7 | SNX4 | | GSTM3 | RPL3 |
| HSPA4 | NONO | SRM | | GSTP1 | RPL30 |
| HSPH1 | NOP2 | SRP54 | | GTF2I | RPL32 |
| IARS | NOP56 | SRP68 | | GTPBP4 | RPL34 |
| IDH3A | NPEPPS | SRSF3 | | H1FX | RPL35 |
| IGF2BP1 | NT5DC1 | SSR4 | | H2AFJ | RPL38 |
| IGF2BP2 | NTPCR | SSRP1 | | H2AFV | RPL4 |
| IGF2BP3 | NUDT5 | STON2 | | H2AFY | RPL5 |
| IGHG1 | NUP93 | STX17 | | H2AFZ | RPL6 |
| IGHG2 | OAT | SUPT16H | | H3F3A | RPL7 |
| IGHG3 | OLA1 | TBL3 | | H3F3B | RPL7A |
| IGHG4 | OSBP | THRAP3 | | HADH | RPLP0 |
| ILF2 | PA2G4 | TIMM44 | | HADHA | RPLP1 |
| IMMT | PAICS | TK1 | | HADHB | RPLP2 |
| IPOS | PARP1 | TMA7 | | HAT1 | RPS10 |
| IQGAP1 | PCBP1 | TMEM33 | | HBB | RPS11 |
| ISOC1 | PCBP2 | TOR1AIP1 | | HBD | RPS13 |
| ITPA | PCDHGA1 | TRIP13 | | HBE1 | RPS14 |
| JUP | PCDHGA10 | TSR1 | | HBG1 | RPS15A |
| KARS | PCDHGA11 | TTC26 | | HBG2 | RPS16 |
| KIAA1468 | PCDHGA12 | TUBA3C | | HDAC1 | RPS17 |
| KIN27 | PCDHGA2 | TUBA4A | | HDAC2 | RPS17L |
| KPNA1 | PCDHGA3 | TUBB4A | | HIST1H2AB | RPS18 |
| KPNA2 | PCDHGA4 | TYMS | | HIST1H2AC | RPS2 |
| KPNA4 | PCDHGA5 | UBA2 | | HIST1H2AD | RPS20 |
| KPNA6 | PCDHGA6 | UBE2G2 | | HIST1H2AG | RPS23 |
| KPNB1 | PCDHGA7 | UHRF1 | | HIST1H2AH | RPS24 |
| KRBA2 | PCDHGA8 | WARS | | HIST1H2AJ | RPS25 |
| KRR1 | PCDHGA9 | XPO5 | | HIST1H3A | RPS27 |
| KTN1 | PCDHGB1 | XRCC5 | | HIST1H4A | RPS27L |
| LARS | PCDHGB2 | ZNF638 | | HIST2H2AA3 | RPS3 |
| LBR | PCDHGB3 | | | HIST2H2AB | RPS3A |
| LDHA | PCDHGB4 | | | HIST2H2AC | RPS4X |
| LDHB | PCDHGB5 | | | HIST2H3A | RPS5 |
| LETM1 | PCDHGB6 | | | HIST3H2A | RPS6 |
| LIG3 | PCDHGB7 | | | HMGB1 | RPS7 |
| LMNB1 | PCDHGC3 | | | HMGB1P1 | RPS8 |
| LMNB2 | PCDHGC4 | | | HMGCS1 | RPS9 |
| LRPPRC | PCDHGC5 | | | HMGN2 | RPSA |
| LRRC47 | PCNA | | | HNRNPA1 | RPSAP58 |
| LTA4H | PDCD10 | | | HNRNPC | RSL1D1 |
| LUC7L | PDHA1 | | | HNRNPD | RSU1 |
| LUC7L2 | PDHB | | | HNRNPM | RUVBL2 |
| MAGOH | PGAM1 | | | HNRNPR | SARS |
| MAGOHB | PGK1 | | | HNRNPU | SEC22B |
| MAP2K2 | PHB | | | HNRNPUL1 | SEC23A |
| MARS | PHB2 | | | HNRNPUL2 | SEPT2 |
| MATR3 | PHGDH | | | HNRNPUL2-BSCL2 | SLC25A5 |
| MCM4 | PKM | | | HPRT1 | SMARCA5 |
| MCM5 | PLEKHJ1 | | | HSD17B10 | SMU1 |
| MCM7 | PM20D2 | | | HSD17B4 | SND1 |
| MDC1 | PNP | | | HSP90AA1 | SNRPA1 |
| MDH1 | POLR1C | | | HSP90AA2 | SNRPD3 |
| ME1 | POTEF | | | HSP90AB1 | SNRPE |
| MMS19 | PPAT | | | HSP90B1 | SPTAN1 |
| MRPL27 | PPIL1 | | | HSPA14 | SRP72 |
| MRPL28 | PPP1CA | | | HSPA4 | STRAP |
| MRPS11 | PPP1CB | | | HSPH1 | TARDBP |
| MRPS17 | PPP1R7 | | | IARS | TCP1 |
| MRPS2 | PRDX1 | | | IDH3A | TMED10 |
| MRPS26 | PRDX2 | | | IGF2BP1 | TMPO |
| MRPS33 | PRDX6 | | | IGF2BP2 | TOMM22 |
| MRPS35 | PRIM2 | | | IGF2BP3 | TOP2A |
| MRPS9 | PRKAG1 | | | IGHG1 | TOP2B |
| MSH6 | PRKDC | | | IGHG2 | TRAP1 |
| MT-CO2 | PRMT1 | | | IGHG3 | TRIM28 |
| MTAP | PRPF19 | | | IGHG4 | TROVE2 |
| MTHFD1 | PRPF3 | | | ILF2 | TSNAX |
| MTR | PRPF4B | | | IMMT | TUBA1A |
| MYBBP1A | PRPS1 | | | IPO5 | TUBA1B |
| MYH9 | PRPSAP2 | | | IQGAP1 | TUBA1C |
| NAA15 | PSMA2 | | | ISOC1 | TUBA8 |
| NACA | PSMA5 | | | ITIH2 | TUBB |
| NAE1 | PSMA7 | | | ITPA | TUBB2A |
| NAMPT | PSMC1 | | | JUP | TUBB2B |
| NAMPTL | PSMC2 | | | KARS | TUBB3 |
| NANS | PSMC4 | | | KIAA1468 | TUBB4B |
| NAPA | PSMC5 | | | KIF15 | TUBB6 |
| NCAPG | PSMC6 | | | KIF5B | TUFM |
| NDUFA10 | PSMD1 | | | KIN27 | U2AF2 |
| NDUFAF4 | PSMD10 | | | KPNA1 | UBA1 |
| NDUFB7 | PSMD11 | | | KPNA2 | UBE2D2 |
| NDUFS1 | PSMD14 | | | KPNA4 | UBE2D3 |
| NDUFS3 | PSMD2 | | | KPNA6 | UBE2O |
| NDUFV1 | PSMD3 | | | KPNB1 | UCHL5 |
| NGDN | PSMD7 | | | KRBA2 | UQCRC1 |
| NHP2 | PTGES2 | | | KRR1 | UQCRC2 |
| NIFK | PTRH2 | | | KTN1 | USO1 |
| NIPSNAP1 | PUF60 | | | LARS | VDAC2 |
| NME1 | PURA | | | LBR | VDAC3 |
| NME1-NME2 | PYCR1 | | | LDHA | WDR5 |
| NME2 | PYCR2 | | | LDHB | WDR61 |
| NOC3L | RAB10 | | | LETM1 | XPO1 |
| NOC4L | RAB11B | | | LIG3 | XRCC6 |
| NONO | RAB1A | | | LMNB1 | YARS |
| NOP2 | RAB1B | | | LMNB2 | YWHAB |
| NPEPPS | RAB5C | | | LRPPRC | YWHAE |
| NTPCR | RAN | | | LRRC47 | YWHAG |
| NUDT5 | RANGAP1 | | | LTA4H | YWHAH |
| NUMA1 | RAP1B | | | LUC7L | YWHAQ |
| OAT | RARS | | | LUC7L2 | YWHAZ |
| OR5AC2 | RAVER1 | | | MAGOH | |
| PA2G4 | RBM14 | | | MAGOHB | |
| PAFAH1B2 | RBM15 | | | MAP2K2 | |
| PAICS | RBM28 | | | MAPRE1 | |
| PARP1 | RBM34 | | | MARS | |
| PCBP1 | RBM39 | | | MATR3 | |
| PCBP2 | RFC4 | | | MCM2 | |
| PCNA | RPA1 | | | MCM3 | |
| PDCD6IP | RPL11 | | | MCM4 | |
| PDHA1 | RPL12 | | | MCM5 | |
| PDHB | RPL13 | | | MCM7 | |
| PDS5A | RPL15 | | | MDC1 | |
| PES1 | RPL18 | | | MDH1 | |
| PFAS | RPL18A | | | ME1 | |
| PGAM1 | RPL19 | | | MMS19 | |
| PGD | RPL22L1 | | | MRPL11 | |
| PGK1 | RPL23 | | | MRPL27 | |
| PGM1 | RPL26 | | | MRPL28 | |
| PGM3 | RPL26L1 | | | MRPL41 | |
| PHB | RPL27 | | | MRPS11 | |
| PHB2 | RPL3 | | | MRPS17 | |
| PHF6 | RPL30 | | | MRPS2 | |
| PHGDH | RPL32 | | | MRPS26 | |
| PKM | RPL34 | | | MRPS33 | |
| PLS3 | RPL35 | | | MRPS35 | |
| POLD3 | RPL38 | | | MRPS9 | |
| POTEF | RPL4 | | | MSH6 | |
| PPAT | RPL5 | | | MT-CO2 | |
| PPP1CA | RPL6 | | | MTAP | |
| PPP1CB | RPL7 | | | MTHFD1 | |
| PPP1CC | RPL7A | | | MTR | |
| PPP1R7 | RPLP0 | | | MYBBP1A | |
| PPP2R1A | RPLP1 | | | MYH10 | |
| PRDX1 | RPLP2 | | | MYH9 | |
| PRDX2 | RPN2 | | | MYL12A | |
| PRDX3 | RPS10 | | | MYL12B | |
| PRDX4 | RPS11 | | | NAA15 | |
| PRDX6 | RPS13 | | | NAA50 | |
| PREP | RPS14 | | | NACA | |
| PRIM2 | RPS15A | | | NAE1 | |
| PRKACA | RPS16 | | | NAMPT | |
| PRKAG1 | RPS17 | | | NAMPTL | |
| PRMT1 | RPS17L | | | NANS | |
| PRMT5 | RPS18 | | | NAPA | |
| PRPF19 | RPS2 | | | NCAPG | |
| PRPF3 | RPS20 | | | NDUFA10 | |
| PRPS1 | RPS23 | | | NDUFA12 | |
| PRPSAP1 | RPS24 | | | NDUFAF4 | |
| PRPSAP2 | RPS25 | | | NDUFB4 | |
| PSMA2 | RPS27 | | | NDUFB7 | |
| PSMA4 | RPS27L | | | NDUFB9 | |
| PSMA8 | RPS3 | | | NDUFS1 | |
| PSMB4 | RPS3A | | | NDUFS3 | |
| PSMC4 | RPS4X | | | NDUFV1 | |
| PSMC5 | RPS5 | | | NGDN | |
| PSMC6 | RPS6 | | | NHP2 | |
| PSMD11 | RPS7 | | | NIFK | |
| PSMD12 | RPS8 | | | NIPSNAP1 | |
| PSMD13 | RPS9 | | | NME1 | |
| PSMD14 | RPSA | | | NME1-NME2 | |
| PSMD3 | RPSAP58 | | | NME2 | |
| PSMD6 | RRBP1 | | | NOC3L | |
| PSMD7 | RRP1B | | | NOC4L | |
| PSME3 | RRP7A | | | NOL7 | |
| PSMG1 | RSL1D1 | | | NONO | |
| PTBP1 | RSU1 | | | NOP2 | |
| PTGES2 | RUVBL2 | | | NOP56 | |
| PTRH2 | SARS | | | NPEPPS | |
| PUF60 | SCO1 | | | NT5DC1 | |
| PURA | SDHB | | | NTPCR | |
| PUS1 | SEC22B | | | NUDT5 | |
| PWP2 | SEC23A | | | NUMA1 | |
| PYCR1 | SEP11 | | | NUP93 | |
| PYGL | SEPT2 | | | OAT | |
| QARS | SF3B1 | | | OLA1 | |
| RAB10 | SF3B3 | | | OR5AC2 | |
| RAB5B | SFRS2 | | | OSBP | |
| RAB5C | SFXN1 | | | PA2G4 | |
| RAB8A | SLC25A4 | | | PAFAH1B2 | |
| RAC1 | SLC25A5 | | | PAICS | |
| RALB | SLC25A6 | | | PARP1 | |
| RALY | SLC3A2 | | | PCBP1 | |
| RAN | SMARCA5 | | | PCBP2 | |
| RANGAP1 | SMU1 | | | PCDHGA1 | |
| RAP1A | SND1 | | | PCDHGA10 | |
| RAP1B | SNRNP35 | | | PCDHGA11 | |
| RAVER1 | SNRPA1 | | | PCDHGA12 | |
| RBBP4 | SNRPD1 | | | PCDHGA2 | |
| RBBP7 | SNRPD3 | | | PCDHGA3 | |
| RBM14 | SNRPE | | | PCDHGA4 | |
| RBM28 | SPTAN1 | | | PCDHGA5 | |
| RBM34 | SRP72 | | | PCDHGA6 | |
| RBM39 | SRSF2 | | | PCDHGA7 | |
| RBM42 | STOML2 | | | PCDHGA8 | |
| RCC1 | STRAP | | | PCDHGA9 | |
| RCC2 | SYNCRIP | | | PCDHGB1 | |
| RFC5 | TALDO1 | | | PCDHGB2 | |
| RHOA | TARDBP | | | PCDHGB3 | |
| RNH1 | TBC1D4 | | | PCDHGB4 | |
| RPA1 | TCEB1 | | | PCDHGB5 | |
| RPA2 | TCP1 | | | PCDHGB6 | |
| RPL10 | TIMM50 | | | PCDHGB7 | |
| RPL10A | TMED10 | | | PCDHGC3 | |
| RPL11 | TMPO | | | PCDHGC4 | |
| RPL12 | TNN | | | PCDHGC5 | |
| RPL13 | TOMM22 | | | PCNA | |
| RPL13A | TOMM70A | | | PDCD10 | |
| RPL14 | TOP2A | | | PDCD6IP | |
| RPL15 | TOP2B | | | PDHA1 | |
| RPL18 | TRAP1 | | | PDHB | |
| RPL18A | TRIM28 | | | PDS5A | |
| RPL19 | TROVE2 | | | PES1 | |
| RPL21 | TSNAX | | | PFAS | |
| RPL22 | TTLL12 | | | PGAM1 | |
| RPL23 | TUBA1A | | | PGD | |
| RPL26 | TUBA1B | | | PGK1 | |
| RPL26L1 | TUBA1C | | | PGM1 | |
| RPL27 | TUBA8 | | | PGM3 | |
| RPL28 | TUBB | | | PHB | |
| RPL3 | TUBB2A | | | PHB2 | |
| RPL30 | TUBB2B | | | PHF6 | |
| RPL32 | TUBB3 | | | PHGDH | |
| RPL34 | TUBB4B | | | PKM | |
| RPL35 | TUBB6 | | | PLEKHJ1 | |
| RPL35A | TUFM | | | PLS3 | |
| RPL37A | U2AF2 | | | PM20D2 | |
| RPL38 | UBA1 | | | PNP | |
| RPL4 | UBE2D2 | | | POLD3 | |
| RPL5 | UBE2D3 | | | POLR1C | |
| RPL6 | UBE2O | | | POTEF | |
| RPL7 | UCHL5 | | | PPAT | |
| RPL7A | UFM1 | | | PPIL1 | |
| RPLP0 | UPF1 | | | PPP1CA | |
| RPLP1 | UQCR10 | | | PPP1CB | |
| RPLP2 | UQCRC1 | | | PPP1CC | |
| RPN1 | UQCRC2 | | | PPP1R7 | |
| RPS10 | USO1 | | | PPP2R1A | |
| RPS11 | VAT1 | | | PRDX1 | |
| RPS13 | VDAC2 | | | PRDX2 | |
| RPS14 | VDAC3 | | | PRDX3 | |
| RPS15A | VTA1 | | | PRDX4 | |
| RPS16 | VTN | | | PRDX6 | |
| RPS17 | WDR36 | | | PREP | |
| RPS17L | WDR5 | | | PRIM2 | |
| RPS18 | WDR61 | | | PRKACA | |
| RPS2 | WDR77 | | | PRKAG1 | |
| RPS20 | WDR82 | | | PRKDC | |
| RPS23 | XPO1 | | | PRMT1 | |
| RPS24 | XRCC6 | | | PRMT5 | |
| RPS25 | YARS | | | PRPF19 | |
| RPS26 | YWHAB | | | PRPF3 | |
| RPS27 | YWHAE | | | PRPF4B | |
| RPS27A | YWHAG | | | PRPS1 | |
| RPS27L | YWHAH | | | PRPSAP1 | |
| RPS28 | YWHAQ | | | PRPSAP2 | |
| RPS3 | YWHAZ | | | PSMA2 | |
| RPS3A | | | | PSMA4 | |
| RPS4X | | | | PSMA5 | |
| RPS5 | | | | PSMA7 | |
| RPS6 | | | | PSMA8 | |
| RPS7 | | | | PSMB4 | |
| RPS8 | | | | PSMC1 | |
| RPS9 | | | | PSMC2 | |
| RPSA | | | | PSMC4 | |
| RPSAP58 | | | | PSMC5 | |
| RSL1D1 | | | | PSMC6 | |
| RSU1 | | | | PSMD1 | |
| RTCB | | | | PSMD10 | |
| RUVBL1 | | | | PSMD11 | |
| RUVBL2 | | | | PSMD12 | |
| SAP30 | | | | PSMD13 | |
| SAR1A | | | | PSMD14 | |
| SAR1B | | | | PSMD2 | |
| SARS | | | | PSMD3 | |
| SCFD1 | | | | PSMD6 | |
| SEC22B | | | | PSMD7 | |
| SEC23A | | | | PSME3 | |
| SEPT2 | | | | PSMG1 | |
| SEPT7 | | | | PTBP1 | |
| SEPT9 | | | | PTGES2 | |
| SF3A2 | | | | PTRH2 | |
| SF3B2 | | | | PUF60 | |
| SFRS3 | | | | PURA | |
| SH3GLB1 | | | | PUS1 | |
| SHMT2 | | | | PWP2 | |
| SLC25A24 | | | | PYCR1 | |
| SLC25A5 | | | | PYCR2 | |
| SLIRP | | | | PYGL | |
| SLTM | | | | QARS | |
| SMARCA5 | | | | RAB10 | |
| SMC1A | | | | RAB11B | |
| SMC3 | | | | RAB1A | |
| SMC4 | | | | RAB1B | |
| SMU1 | | | | RAB5B | |
| SND1 | | | | RAB5C | |
| SNRNP200 | | | | RAB8A | |
| SNRPA1 | | | | RAC1 | |
| SNRPB | | | | RALB | |
| SNRPD3 | | | | RALY | |
| SNRPE | | | | RAN | |
| SNRPN | | | | RANGAP1 | |
| SNX15 | | | | RAP1A | |
| SNX4 | | | | RAP1B | |
| SPTAN1 | | | | RARS | |
| SRM | | | | RAVER1 | |
| SRP54 | | | | RBBP4 | |
| SRP68 | | | | RBBP7 | |
| SRP72 | | | | RBM14 | |
| SRSF3 | | | | RBM15 | |
| SSR4 | | | | RBM28 | |
| SSRP1 | | | | RBM34 | |
| STON2 | | | | RBM39 | |
| STRAP | | | | RBM42 | |
| STX17 | | | | RCC1 | |
| SUPT16H | | | | RCC2 | |
| TARDBP | | | | RFC4 | |
| TBL3 | | | | RFC5 | |
| TCP1 | | | | RHOA | |
| THRAP3 | | | | RNH1 | |
| TIMM44 | | | | RPA1 | |
| TK1 | | | | RPA2 | |
| TMA7 | | | | RPL10 | |
| TMED10 | | | | RPL10A | |
| TMEM33 | | | | RPL11 | |
| TMPO | | | | RPL12 | |
| TOMM22 | | | | RPL13 | |
| TOP2A | | | | RPL13A | |
| TOP2B | | | | RPL14 | |
| TOR1AIP1 | | | | RPL15 | |
| TRAP1 | | | | RPL18 | |
| TRIM28 | | | | RPL18A | |
| TRIP13 | | | | RPL19 | |
| TROVE2 | | | | RPL21 | |
| TSNAX | | | | RPL22 | |
| TSR1 | | | | RPL22L1 | |
| TTC26 | | | | RPL23 | |
| TUBA1A | | | | RPL26 | |
| TUBA1B | | | | RPL26L1 | |
| TUBA1C | | | | RPL27 | |
| TUBA3C | | | | RPL28 | |
| TUBA4A | | | | RPL3 | |
| TUBA8 | | | | RPL30 | |
| TUBB | | | | RPL32 | |
| TUBB2A | | | | RPL34 | |
| TUBB2B | | | | RPL35 | |
| TUBB3 | | | | RPL35A | |
| TUBB4A | | | | RPL37A | |
| TUBB4B | | | | RPL38 | |
| TUBB6 | | | | RPL4 | |
| TUFM | | | | RPL5 | |
| TYMS | | | | RPL6 | |
| U2AF2 | | | | RPL7 | |
| UBA1 | | | | RPL7A | |
| UBA2 | | | | RPLP0 | |
| UBE2D2 | | | | RPLP1 | |
| UBE2D3 | | | | RPLP2 | |
| UBE2G2 | | | | RPN1 | |
| UBE2O | | | | RPN2 | |
| UCHL5 | | | | RPS10 | |
| UHRF1 | | | | RPS11 | |
| UQCRC1 | | | | RPS13 | |
| UQCRC2 | | | | RPS14 | |
| USO1 | | | | RPS15A | |
| VDAC2 | | | | RPS16 | |
| VDAC3 | | | | RPS17 | |
| WARS | | | | RPS17L | |
| WDR5 | | | | RPS18 | |
| WDR61 | | | | RPS2 | |
| XPO1 | | | | RPS20 | |
| XPO5 | | | | RPS23 | |
| XRCC5 | | | | RPS24 | |
| XRCC6 | | | | RPS25 | |
| YARS | | | | RPS26 | |
| YWHAB | | | | RPS27 | |
| YWHAE | | | | RPS27A | |
| YWHAG | | | | RPS27L | |
| YWHAH | | | | RPS28 | |
| YWHAQ | | | | RPS3 | |
| YWHAZ | | | | RPS3A | |
| ZNF638 | | | | RPS4X | |
| | | | | RPS5 | |
| | | | | RPS6 | |
| | | | | RPS7 | |
| | | | | RPS8 | |
| | | | | RPS9 | |
| | | | | RPSA | |
| | | | | RPSAP58 | |
| | | | | RRBP1 | |
| | | | | RRP1B | |
| | | | | RRP7A | |
| | | | | RSL1D1 | |
| | | | | RSU1 | |
| | | | | RTCB | |
| | | | | RUVBL1 | |
| | | | | RUVBL2 | |
| | | | | SAP30 | |
| | | | | SAR1A | |
| | | | | SAR1B | |
| | | | | SARS | |
| | | | | SCFD1 | |
| | | | | SCO1 | |
| | | | | SDHB | |
| | | | | SEC22B | |
| | | | | SEC23A | |
| | | | | SEPT11 | |
| | | | | SEPT2 | |
| | | | | SEPT7 | |
| | | | | SEPT9 | |
| | | | | SF3A2 | |
| | | | | SF3B1 | |
| | | | | SF3B2 | |
| | | | | SF3B3 | |
| | | | | SFRS2 | |
| | | | | SFRS3 | |
| | | | | SFXN1 | |
| | | | | SH3GLB1 | |
| | | | | SHMT2 | |
| | | | | SLC25A24 | |
| | | | | SLC25A4 | |
| | | | | SLC25A5 | |
| | | | | SLC25A6 | |
| | | | | SLC3A2 | |
| | | | | SLIRP | |
| | | | | SLTM | |
| | | | | SMARCA5 | |
| | | | | SMC1A | |
| | | | | SMC3 | |
| | | | | SMC4 | |
| | | | | SMU1 | |
| | | | | SND1 | |
| | | | | SNRNP200 | |
| | | | | SNRNP35 | |
| | | | | SNRPA1 | |
| | | | | SNRPB | |
| | | | | SNRPD1 | |
| | | | | SNRPD3 | |
| | | | | SNRPE | |
| | | | | SNRPN | |
| | | | | SNX15 | |
| | | | | SNX4 | |
| | | | | SPTAN1 | |
| | | | | SRM | |
| | | | | SRP54 | |
| | | | | SRP68 | |
| | | | | SRP72 | |
| | | | | SRSF2 | |
| | | | | SRSF3 | |
| | | | | SSR4 | |
| | | | | SSRP1 | |
| | | | | STOML2 | |
| | | | | STON2 | |
| | | | | STRAP | |
| | | | | STX17 | |
| | | | | SUPT16H | |
| | | | | SYNCRIP | |
| | | | | TALDO1 | |
| | | | | TARDBP | |
| | | | | TBC1D4 | |
| | | | | TBL3 | |
| | | | | TCEB1 | |
| | | | | TCP1 | |
| | | | | THRAP3 | |
| | | | | TIMM44 | |
| | | | | TIMM50 | |
| | | | | TK1 | |
| | | | | TMA7 | |
| | | | | TMED10 | |
| | | | | TMEM33 | |
| | | | | TMPO | |
| | | | | TNN | |
| | | | | TOMM22 | |
| | | | | TOMM70A | |
| | | | | TOP2A | |
| | | | | TOP2B | |
| | | | | TOR1AIP1 | |
| | | | | TRAP1 | |
| | | | | TRIM28 | |
| | | | | TRIP13 | |
| | | | | TROVE2 | |
| | | | | TSNAX | |
| | | | | TSR1 | |
| | | | | TTC26 | |
| | | | | TTLL12 | |
| | | | | TUBA1A | |
| | | | | TUBA1B | |
| | | | | TUBA1C | |
| | | | | TUBA3C | |
| | | | | TUBA4A | |
| | | | | TUBA8 | |
| | | | | TUBB | |
| | | | | TUBB2A | |
| | | | | TUBB2B | |
| | | | | TUBB3 | |
| | | | | TUBB4A | |
| | | | | TUBB4B | |
| | | | | TUBB6 | |
| | | | | TUFM | |
| | | | | TYMS | |
| | | | | U2AF2 | |
| | | | | UBA1 | |
| | | | | UBA2 | |
| | | | | UBE2D2 | |
| | | | | UBE2D3 | |
| | | | | UBE2G2 | |
| | | | | UBE2O | |
| | | | | UCHL5 | |
| | | | | UFM1 | |
| | | | | UHRF1 | |
| | | | | UPF1 | |
| | | | | UQCR10 | |
| | | | | UQCRC1 | |
| | | | | UQCRC2 | |
| | | | | USO1 | |
| | | | | VAT1 | |
| | | | | VDAC2 | |
| | | | | VDAC3 | |
| | | | | VTA1 | |
| | | | | VTN | |
| | | | | WARS | |
| | | | | WDR36 | |
| | | | | WDR5 | |
| | | | | WDR61 | |
| | | | | WDR77 | |
| | | | | WDR82 | |
| | | | | XPO1 | |
| | | | | XPO5 | |
| | | | | XRCC5 | |
| | | | | XRCC6 | |
| | | | | YARS | |
| | | | | YWHAB | |
| | | | | YWHAE | |
| | | | | YWHAG | |
| | | | | YWHAH | |
| | | | | YWHAQ | |
| | | | | YWHAZ | |
| | | | | ZNF638 | |

Examples of polypeptides including a histidine phosphorylated at N3 are presented in above. One or more of these polypeptides including a histidine phosphorylated at N3 can be detected in the methods disclosed herein, such as for detecting a tumor or determining the effectiveness of a chemotherapeutic agent. In some embodiments, 1, 2, 3, 4, 5, 6, 7, 8, 9, or 10 of the listed polypeptides can be detected. In other embodiments, 5, 10, 15, 20, 25, 30, 35, 40, 45 or 50 of these polypeptides can be detected. Any combination of the polypeptides listed in Tables 1 and/or Table 2 can be used in the presently disclosed methods. In some embodiments, the methods utilize any combination of the polypeptides listed in Table 2. In some examples a combination of any of the listed “pHis3 only” phosphorylated polypeptides is utilized.

### Example 14

### Materials and Methods

Materials and Chemicals:

Reagents and their sources were as follows: FLAG-NME1/Nm23-H1 mammalian expression vector was from Addgene (Cat. 25000), GST-PGAM1 (Cat. H00005223-P01) was from Novus Biological, pGEX-6P-1 GST-fusion vector (Cat. 28-9546-48) and PreScission Protease (27-0843-01) were from GE Life Sciences, Rosetta™ 2 (DE3) competent cells (Cat. 71397), the pTyr mAb clone 4G10 (Cat. 05-321) was from EMD Millipore, 2,3-diphospho-D-glyceric acid pentasodium salt (Cat. SC-213964) and NME1 mAb (Cat. SC-136141) were from Santa Cruz, Alexa Fluor® 680 goat anti-rabbit IgG secondary antibody (Cat. A-21109), GST mAb (Cat. 13-6700), Oregon Green-Dextran®488 and LysoTracker Red DND-99 (Cat. L-7528) were from Life Technologies, goat anti-mouse IgG (H+L) secondary antibody, DyLight 800 conjugate (Cat. 35521) was from Pierce and Casein Blocking Buffer was from BioRad. Amicon Ultrafree 0.5-5K MWCO centrifugal filters (Cat. UFC500396), Immobilon-FL PVDF membranes (Cat. IPFL00010), the Mini-PROTEAN II Multiscreen Apparatus (Cat. 170-4017) and Casein blocking solution (Cat. 161-0783) was from BioRad. Glutathione resin (Cat. L00206) was from Genscript, Ampicillin, chloramphenicol, Adenosine 5′-triphosphate disodium salt (Cat. A2383), SDS, Trizma base, glycine, Isopropyl β-D-1-thiogalactopyranoside (Cat. 16758), Anti-FLAG M2 mAb (Cat. F1804), Anti-α-tubulin (Cat. T5168), Anti-γ-tubulin (T6557), Freund's Complete Adjuvant (Cat. F5881), Freund's Incomplete Adjuvant (Cat. F5506), iodoacetamide and octyl-β-D-glucopyranosideside (Cat. 08001) were from Sigma-Aldrich. Anti-Aurora A mAb was from Abcam (Ab13824). The SulfoLink Coupling Resin (Cat. 44999), the chemical crosslinkers DSS (Cat. 21555) and BS3 (Cat. 21580) and SILAC reagents (Cat. 89983 and 89990) were from Pierce/Thermo Scientific. All protein electrophoresis equipment including; Four Gel Caster (Cat. SE275), Mighty Small II Mini Deluxe Vertical Unit (Cat. SE260-10A-0.75), Mighty Small Mini Transfer Tank (Cat. TE22) were from Hoefer, RPMI 1640 and 1% Glutamax (Cat. 35050-061) were from Gibco/Life technologies (Cat. 11875-119), Rabbit Hybridoma Supplement A was from Epitomics/AbCam (Cat. EP-401), cell culture grade 55 mM 2-Mercaptoethanol was from Invitrogen (Cat. 2198-023), IS-MAB-CD Serum-free medium was from Irvine Scientific (Cat. 91104) and 1% antibiotic/antimycotic solution was from Cellgro (Cat. 30-0004-CI). Tris(2-carboxyethyl)phosphine hydrochloride (TCEP) was from Roche Applied Science. Paraformaldehyde (PFA) was from Electron Microscopy Sciences (Cat. 15710).

Immunization of Rabbits

New Zealand White rabbits (three per antigen) were immunized using a standard prime-boost regimen and pre-immune serum was collected from each rabbit prior to immunization. The pTza peptide libraries were coupled to KLH, diluted to 1 mg/ml and stored in 1.5 ml aliquots at −20° C. For the primary immunization, Freund's Complete Adjuvant (0.5 ml per rabbit) was emulsified with antigen (0.5 ml per rabbit) using 5 ml syringes. Antigens were administered via intradermal injection of 50 μl at 20 sites on the back. Every three weeks, subsequent boosts were administered intradermally in Incomplete Freund's Adjuvant. Bleeds were collected in 10 ml tubes from the central ear artery ten days after each boost. Rabbit antisera was collected after spinning down blood (2,400×g for 10 min at 4° C.) that was allowed to clot for 24-48 hr. Antisera was frozen at −20° C. for long term storage.

Synthetic Peptide Synthesis:

Sequences of synthetic peptides and pTyr phosphopeptides used in this study are as follows; Nck pY105 (CGERLpYDLNMPAYVK (SEQ ID NO; 55), Nck Y105 (CGERLYDLNMPAYVK (SEQ ID NO: 56)), Eck (EphA2) pY588 (CLKPLKTpYVD (SEQ ID NO: 57)), Eck (EphA2) Y588 (CLKPLKTYVD (SEQ ID NO: 58)) and FAK pY397 (AVSVSETDDpYAEIIDEEDTYT (SEQ ID NO: 59)). Peptides were synthesized using Fmoc solid phase synthesis.

Peptide Dot Blot Screening of Rabbit Antisera:

Peptide dot blots were used initially to screen rabbit antisera titer. The 1-pTza and 3-pTza peptide libraries, His control library and a pTyr peptide (Nck pY105) were dissolved in water at a stock concentration of 1 mg/ml. 1:5 serial dilutions (500, 100, 20, 5, 1 and 0.2 ng/ul) were prepared for each peptide and 1 ul of each dilution was spotted on nitrocellulose membrane and allowed to dry for 1-2 hr at RT. Membranes were blocked for 1 hr at RT in Casein Blocking Buffer (0.1% casein, 0.2×PBS −/−) and incubated with rabbit antisera or pre-immune serum (diluted 1:1,000 in Blocking Buffer with 0.1% Tween-20) for 1 hr at RT or overnight at 4° C. All subsequent steps were as described for “immunoblotting with anti-pHis antibodies”.

Protein Expression and Purification:

NME1, NME2 and PGAM were subcloned into the pGEX-6P-1 GST-fusion vector. The following primers were used for PCR amplification and insertion of BamHI and EcoRI restriction sites;

NME1-Fw,
(SEQ ID NO: 60)
5′-GATCGGATCCATGGCCAACTGTGAGCGTAC-3′,
NME1-Rev,
(SEQ ID NO: 61)
5′-GATCGAATTCTCATTCATAGATCCAGTTCTC-3′,,
NME2-Fw,
(SEQ ID NO: 62)
5′-GATCGGATC-CATGGCCAACCTGGAGCGCAC-3′,
NME2-Rev,
(SEQ ID NO: 63)
5′-GATCGAATTCTTATTCATAGAC-CCAGTCATG-3′,
and
PGAM-Fw,
(SEQ ID NO: 64)
5′-GATCGGATCCATGGCCGCCTACAAACTGGTG-3′,
PGAM-Rev-
(SEQ ID NO: 65)
5′-GATCGAATTCTCACTTCTTGGCCTTGCCCTG-3′.

ROSETTA™ 2 (DE3) competent cells were transformed with pGEX-NME1, pGEX-NME2 or pGEX-PGAM and starter cultures from single colonies were grown at 37° C. for 16 hr in LB broth supplemented with 100 ug/ml ampicillin and 34 ug/ml chloramphenicol with shaking at 225 RPM. Expression cultures were diluted from starter cultures with the same medium to an A600 of 0.2. Protein expression was induced with 1 mM IPTG at an A600 of 0.6 for 3 hr at 30° C. Bacteria were pelleted (5,000×g for 10 min at 4° C.) and resuspended in 1 ml GST Lysis/Wash Buffer (PBS, pH 8.0, 1% Triton X-100, 5% glycerol, 1 mM DTT)/50 ml culture. Lysates were sonicated on ice and clarified by centrifugation (14,000×g for 30 min at 4° C.). Glutathione resin was equilibrated with GST lysis/wash buffer and 1 ml washed resin/200 ml culture was incubated with clarified bacterial lysates for 2 hr at 4° C. Resin was then pelleted and the supernatant was removed before washing at least 3 times with 10 ml wash buffer. Washed resin was resuspended in 2 ml PreScission Protease Buffer (20 mM Tris pH=7.0, 150 mM NaCl, 1 mM DTT, 0.5 mM EDTA) and cleavage of GST-tag was performed overnight at 4° C. using 2 ul PreScission Protease (5U/200 ml culture). Cleaved resin was pelleted (1000×g for 5 min at 4° C.) and supernatants were transferred to fresh tubes. Buffer exchange into Storage Buffer B (50 mM Tris-HCl, pH 8.0, 100 mM NaCl, 1 mM DTT) was performed using centrifugal filters and proteins were concentrated to ˜1 ug/ul. Purified proteins were supplemented with 10% glycerol and stored at −80° C. Quantification of purified proteins was performed by densitometry of Coomassie stained gels using a BSA standard curve.

NME and PGAM In Vitro Phosphorylation Assays:

In vitro autophosphorylation of purified NME1 and NME2 (10-30 ng/ul) was performed in TMD buffer (20 mM Tris-HCl, pH 8.0, 5 mM MgCl₂, 1 mM DTT) at RT. Fresh ATP was added to initiate reactions which were allowed to proceed at RT for 10 min. Reactions were stopped by addition of 5×pH 8.8 sample buffer and analyzed immediately by SDS-PAGE (see immunoblotting with anti-pHis antibodies). Reactions lacking ATP or treated briefly with heat or acid served as negative controls. In vitro autophosphorylation of PGAM was performed as described for NME1 except 2,3-diphosphoglycerate (2,3-DPG) was used as the phosphate donor instead of ATP and incubations were carried out at 30° C. Reactions lacking 2,3-DPG or treated briefly with heat served as negative controls. Heat treatment was performed after addition of 5×pH 8.8 sample buffer for 10-15 min at 95° C. Acid treatment was performed by adding 25 ul 1N HCl to a 100 ul reaction and incubating at 37° C. for 15 min. Reactions were neutralized with 25 ul 1 N NaOH before addition of 5×pH 8.8 sample buffer.

Immunoblotting with Anti-pHis Antibodies:

In general, immunoblotting with anti-pHis antibodies was performed with modifications to standard procedures to help preserve pHis for detection. Buffers were adjusted to pH 8-9 to stabilize pHis and methods were modified to avoid heating samples. Protein samples were prepared in pH 8.8 sample buffer (5×=10% SDS, 250 mM Tris-HCl, pH 8.8, 0.02% Bromophenol blue, 50% glycerol, 50 mM EDTA, 500 mM DTT) for electrophoresis. Mammalian whole cell lysates were prepared by rinsing 70-100% confluent 10 cm² dishes twice with 5 ml cold TD buffer (TBS −/−, pH 8). Cells were scraped directly into 2×pH 8.8 sample buffer, incubated on ice and a cup horn sonicator was used (3-5×5 sec bursts) to disrupt cells and shear DNA. Lysates were clarified by centrifugation (14,000×g for 5-15 min at 4° C.) and analyzed immediately using freshly prepared Bis-Tris polyacrylamide minigels with a modified, pH 8.8 stacking gel and either 10% or 12.5% resolving gels. Electrophoresis buffer recipes were as follows: Running Buffer: (1×20 L, pH 8.5) 20 g SDS, 60 g Trizma Base, 288 g glycine, dH₂0 to 20 L, Transfer Buffer: (1×4 L, pH 8.5) 56.7 g glycine, 4 g SDS, 12 g Trizma Base, 800 ml MeOH, dH₂0 to 4 L. All electrophoresis steps were performed at 4° C. and samples were resolved at 90-100V for 2-3 hr. Proteins were transferred to Immunoblon-FL PVDF membranes at 30V for 12-18 hr at 4° C. and immediately incubated for 45-60 min at RT or >2 hr at 4° C. in Casein Blocking Buffer (0.1% casein, 0.2×PBS −/−). Primary antibodies were diluted in blocking buffer with 0.1% Tween-20, incubated with membranes for 1 hr at RT, or 3-18 hr at 4° C. Membranes were washed at least three times for 10 min each with 0.1% TBST before incubation with secondary antibodies for 45-50 min at room temperature. Rabbit anti-pHis antisera was stored at −20° C. was used at 1:1,000 for dot blots and western blots. Affinity purified, polyclonal anti-pHis antibodies were stored at 4° C. and used at 1:200. After incubation with secondary antibodies, membranes were washed least four times for 10 min each with 0.1% TBST. Immunoblots and Coomassie stained gels were imaged on a LI-COR Odyssey Infrared Imaging System. Duplexing of primary antibodies was performed using both channels of the Odyssey by co-incubating membranes with rabbit primary antibodies with mouse; anti-FLAG-M2, anti-GST or anti-NME1. For rabbit antibodies, Alexa Fluor® 680 Goat Anti-Rabbit IgG secondary antibodies were diluted 1:20,000 in blocking buffer supplemented with 0.1% tween-20 and 0.01% SDS. For mouse primary antibodies, Goat Anti-Mouse IgG secondary antibody (DyLight 800 conjugate) were diluted 1:20,000 in blocking buffer supplemented with 0.1% tween-20 and 0.01% SDS and incubated alone or co-incubated with Alexa Fluor® 680 Goat Anti-Rabbit secondary antibodies for duplexed primary antibodies.

Slot Blot Screening of Hybridoma Cell Supernatants:

A slot blotting apparatus was used to screen up to 40 anti-pHis hybridoma cell supernatants simultaneously. Preparative slab gels were cast using custom Teflon combs to create stacking gels that contained one large sample well and a single lane for loading protein molecular weight standards. SDS-PAGE was performed as described above for immunoblotting with anti-pHis antibodies. Briefly, PVDF membranes were clamped into the BioRad Miniprotean II Multiscreen Apparatus and blotting was performed as instructed by the manufacturer. IgG concentrations of hybridoma cell-supernatants (obtained from IgG ELISA assays performed by Epitomics) were normalized to 0.5 ug/ml for screening of anti-pHis mAbs (dilution factors ranged from 1:5 to 1:500) by dilution with casein blocking buffer supplemented with 0.1% Tween-20. 600 ul of each diluted mAb was pipetted into each chamber and incubated on top of the membrane for at 4° C. for 3 hr. After three 10 min washes of the membranes in the apparatus with 0.1% TBST, the membranes were transferred to blotting containers and washed again using larger volumes of 0.1% TBST. Incubation with secondary antibodies imaging was performed as described above.

Affinity Purification of Polyclonal pHis Antibodies:

Affinity columns for purification of polyclonal antibodies from rabbit antisera were prepared by covalently coupling 2 mg of either PEG-1-pTza or PEG-3-pTza peptide libraries to 2 ml SulfoLink agarose resin according to manufacturer's instructions. 5 ml of the corresponding anti-1-pTza or anti-3-pTza antisera was thawed on ice and diluted 1:2 with PBS (pH 7.4). Diluted antiserum was clarified by centrifugation (8,000×g for 20 min at 4° C.) and a sample was taken for analysis (“Input”). The columns were equilibrated with 15 ml PBS and the clarified antiserum was passed over columns three times. The flow through (FT) was collected and the column was then washed twice with PBS (15 ml=“Wash 1”, 1 ml=“Wash 2”). The antibodies were eluted by addition of 0.1 M glycine (pH 2.5) buffer and 15×1 ml and 4×2 ml elution fractions were collected and immediately neutralized to pH 7 with sodium phosphate. A final wash step was performed with PBS (15 ml=“Wash 3”, 1 ml=“Wash 4”). Samples from each elution and wash fraction and a 10 ul sample of column material (“col”) were saved for SDS-PAGE analysis followed by Coomassie staining (FIG. 3D) to monitor binding and elution of IgG. Elution fractions were also tested for anti-3-pHis antibodies by immunoblotting in vitro phosphorylated PGAM (FIG. 3E).

pTza Peptide Dot Blot Arrays:

1-pTza, 3-pTza or His was incorporated into synthetic peptides of defined sequences from mammalian proteins with mapped pHis sites. The peptides used were as follows: ACLY-like H760 (AGAG-X-AGAG, SEQ ID NO: 89), PGAM H11 (VLIR-X-GESA, SEQ ID NO: 90), NME1 H118 (RNII-X-GSDS, SEQ ID NO: 91), Histone H4 H18 (GAKR-X-RKVL, SEQ ID NO: 92), KCa3.1 H358 (VRLK-X-RKLR, SEQ ID NO: 93) and GNB1 H266 (MTYS-X-DNII, SEQ ID NO: 94) where X=His, 1-pTza or 3-pTza. Peptides were dissolved in water at a stock concentration of 1 mg/ml. 1:5 serial dilutions (500, 100, 20, 5, 1 and 0.2 ng/ul) were prepared for each peptide and 1 ul of each dilution was spotted on nitrocellulose membrane and allowed to dry for 1-2 hr at RT. The pTza peptide stock solutions had a pH of 4-5 so these were neutralized by addition of 25 ul of 1M Tris buffer pH 8.0. Immunoblotting was performed as described above.

Cell Culture and Stable Cell Line Generation:

Human embryonic kidney cells (HEK 293), HeLa, Psrc11 and pancreatic stellate cells (PaSCs) were cultured in a 37° C., 5.0% CO₂ incubator. HEK 293 and HeLa cells were grown in DMEM (4.5 g/liter glucose, L-glutamine, and sodium pyruvate) supplemented with 10% FBS without antibiotics. Prsc11 were grown in DMEM supplemented with 4% FCS and Pen/Strep.

For generation of stably transfected FLAG-NME1 cell lines, HEK 293 cells were transiently transfected with 15 ug FLAG-NME1 mammalian expression vector in a 10 cm² dish using the calcium phosphate method. 48 hr post-transfection cells were split and plated in 96-well plates and stable transfectants were selected with G418. After 10 days in selection medium, surviving clones were trypsinized and expended in 6-well plates. Single colonies were selected, expanded and cell lysates were analyzed by immunoblotting with anti-NME1 and anti-FLAG antibodies to confirm stable integration.

Rabbit Hybridoma Cell Culture:

pHis hybridoma cell lines were maintained with Growth Medium (1×HAT 240E medium; 500 ml RPMI 1640, 40 ml Rabbit Hybridoma Supplement A (Epitomics), 55 μM 2-Mercaptoethanol and 10% FBS) in a 37° C., 5% CO₂ incubator. Briefly, cultures were seeded at 1×10⁵ cells/ml and split at 70-80% confluency by aspirating media and replacing with fresh medium. Cell lines were stored in liquid N₂ in freezing media (90% FBS, 10% DMSO).

Sequencing pHis Antibody IgG V_{H} and V_{L} Regions:

Anti-1-pHis and Anti-3-pHis hybridomas were cultured as described above and ˜750,000 cells were collected by centrifugation at 1,100 RPM for 5 min. 20-30 ug RNA was isolated from each hybridoma using the Qiagen RNA Easy Mini Kit according to the manufacturer's instructions. RT-PCR was performed to using the Superscript III First-Strand Synthesis System (Life Technologies Cat. 18080-051) to synthesize cDNA from RNA primed with oligo(dT) primers. PCR primers used to amplify and sequence IgG V_{H} and V_{L} regions are listed. (SEQ ID NOs. 66-88)

TABLE 3
Rabbit V_{H} V_{L} primers
Light Chain

| | |
|---|---|
| Vk1_A | 5′ GTGATGACCCAGACTCCA 3′ |
| Vk1_C | 5′ GTGCTGACCCAGACTCCA 3′ |
| Vk2_A | 5′ GATATGACCCAGACTCCA 3′ |
| Vk2_C | 5′ GATCTGACCCAGACTCCA 3′ |
| vk3 | 5′ TTTGATTTCCACATTGGTGCC 3′ |
| vk4 | 5′ TAGGATCTCCAGCTCGGTCCC 3′ |
| vk5_C | 5′ TTTGACCACCACCTCGGTCCC 3′ |
| vk5_G | 5′ TTTGACGACCACCTCGGTCCC 3′ |
| Vλ1 | 5′ GTGCTGACTCAGTCGCCCTC 3′ |
| vλ2 | 5′ GCCTGTGACGGTCAGCTGGGTCCC 3′ |

Heavy Chain

| | |
|---|---|
| VH1_A | 5′ AGTCGGTGGAGGAGTCCAGG 3′ |
| VH1_G | 5′ AGTCGGTGGAGGAGTCCGGG 3′ |
| VH2 | 5′ AGTCGGTGAAGGAGTCCGAG 3′ |
| VH3_C | 5′ AGTCGCTGGAGGAGTCCGGG 3′ |
| VH3_T | 5′ AGTCGTTGGAGGAGTCCGGG 3′ |
| VH4_CA | 5′ AGCAGCAGCTGATGGAGTCCGG 3′ |
| VH4_GA | 5′ AGGAGCAGCTGATGGAGTCCGG 3′ |
| VH4_CG | 5′ AGCAGCAGCTGGTGGAGTCCGG 3′ |
| VH4_GG | 5′ AGGAGCAGCTGGTGGAGTCCGG 3′ |
| vh5_AC | 5′ AGAGACGGTGACCAGGGTGCC 3′ |
| vh5_GC | 5′ GGAGACGGTGACCAGGGTGCC 3′ |
| vh5_AT | 5′ AGAGATGGTGACCAGGGTGCC 3′ |
| vh5_GT | 5′ GGAGATGGTGACCAGGGTGCC 3′ |

cDNA from RT-PCR reactions was analyzed by gel electrophoresis and reactions yielding products of the correct size (300-350 bp) were sequenced with both forward and reverse primers.

pHis mAb Production and Purification:

pHis hybridomas were expanded from 10 cm2 dishes to T175 flasks in 60 ml Growth Medium. Once confluent, cells were collected by centrifugation at 1,100 RPM for 5 min in 2×50 mL tubes. 22.5 ml supernatant was removed from each tube and cells were resuspended in the remaining 2×7.5 ml medium. Cells were transferred back into the same T175 flask and 45 ml fresh Serum-Free Medium (SFM; IS-MAB CD chemically defined medium (Irvine Scientific), 1% antibiotic/antimycotic supplement and 1% Glutamax) was added. Cells were acclimated to this low-serum (2.5%) condition for 3 days. Cells were spun, as before, into 2×50 ml tubes and all media was aspirated from pellets. Cells were resuspended in 2×7.5 ml SFM and transferred back into their respective T175 flasks with 45 ml (60 ml total) SFM. Cells were grown in SFM until cell viability was approximately 50% (˜7-10 days). To harvest antibodies, cells were collected by centrifugation. Cell supernatants were spun again in fresh tubes at 3,000 RPM for an additional 15 min. For antibody purification, 1 ml Protein-A-agarose beads were incubated overnight at 4° C. with 50 ml SFM hybridoma cell supernatant. The Protein-A-agarose beads were pelleted at 4,000×g for 5 min at 4° C. and washed with 3× with 10 ml PBS (pH 7.4). Anti-pHis IgG was eluted with two sequential additions of 1 ml Elution Buffer (200 mM Glycine, pH 2.8), which were immediately neutralized with 1.0 M Tris-HCl (pH 8.3). Anti-pHis mAb concentrations were measured by IgG A280 and stored at 4° C. Purified mAbs were used at a concentration of 0.5 ug/ml (1:2000) and validated by immunoblotting cell lysates and dot blotting in vitro phosphorylated NME1 (1-pHis) or PGAM (3-pHis).

Mass Spectrometry-Detection of pHis Sites on NME1 and PGAM:

In vitro phosphorylated NME1 and PGAM samples were first denatured in 8 M urea and then reduced and alkylated with 10 mM TCEP and 55 mM iodoacetamide respectively. The samples were diluted to 2 M urea with 100 mM Tris pH 8.5 and then digested with trypsin [Promega] at room temperature for 4 hours.

Each protein digest was pressure-loaded onto 250 micron i.d. fused silica capillary [Polymicro Technologies] columns with a Kasil frit packed with 3 cm of 5 micron C18 resin [Phenomenex]. After desalting, each column was connected to a 100 micron i.d. fused silica capillary [Polymicro Technologies] analytical column with a 5 micron pulled-tip, packed with 10 cm of 5 micron C18 resin [Phenomenex].

Each column was placed inline with an Easy NanoLC II pump [Thermo Scientific] and the eluted peptides were electrosprayed directly into a Q Exactive mass spectrometer [Thermo Scientific]. The buffer solutions used were 10 mM ammonium bicarbonate pH 5 (buffer A) and 100% methanol (buffer B). The 90 minute elution gradient had the following profile: 10% buffer B at 5 minutes, to 55% buffer B at 50 minutes, to 99% buffer B at 65 minutes and continuing to 75 minutes. A cycle consisted of one full scan mass spectrum (400-1600 m/z) at 70 K resolution followed by up to 10 data-dependent MS/MS (fixed first mass, 100 m/z) at 17.5 K resolution using a normalized collision energy (NCE) of 25 with 20% stepped NCE. Charge state exclusion was selected such that only +2 and +3 ions were selected for fragmentation. Dynamic exclusion was set at 10 seconds. Application of mass spectrometer scan functions and HPLC solvent gradients were controlled by the Xcalibur data system [Thermo Scientific].

MS/MS spectra were extracted using RawXtract (version 1.9.9) (McDonald et al., Rapid Commun Mass Spectrom. 18:2162-21682004 (2004)). MS/MS spectra were searched with the ProLuCID algorithm (Eng et al., J Am Soc Mass Spectrom. 5: 976-989, (1994)) against a Saccharomyces cerevisiae and Escherichia coli database concatenated to a decoy database in which the sequence for each entry in the original database was reversed (Peng et al., J Proteome Res. 2: 43-50 (2003)) supplemented with UniProt sequences for either human NM23 or human PGAM1. The ProLuCID search was performed using full enzyme specificity, static modification of cysteine due to carboxyamidomethylation (57.02146) and differential modification of histidine, serine, threonine and tyrosine due to phosphorylation (79.9663). The data was searched using a precursor mass tolerance of 50 ppm and a fragment ion mass tolerance of 10 ppm. ProLuCID search results were assembled and filtered using the DTASelect (version 2.0) algorithm (Tabb et al., J Proteomics Res. 1:21-26, (2002)). All peptide-spectra matches had less than 10 ppm mass error. Phosphorylation site assignment was confirmed by manual annotation of spectra.

Stable Isotope Labeling (SILAC) and pHis mAb Immunoaffinity Purification of Proteins for LC-MS/MS:

1-pHis mAb SC1-1 and 3-pHis mAb SC39-4 affinity resins were generated by crosslinking purified mAbs to protein-A agarose beads using DSS or BS3. mAbs were coupled to beads at 1 mg IgG/ml of protein-A beads. After crosslinking, pHis mAb resins (˜750 μl each) were packed in 10 ml chromatography columns (BioRad) and stored at 4° C. in or equilibrated with Wash/Binding Buffer (50 mM Tris, 30 mM sodium carbonate pH 8 (prepared by dilution of 100 mM sodium carbonate buffer pH 10 with H₂O and 1 M Tris pH 7)). Stable isotope labeling by amino acids in cell culture (SILAC) was performed on FLAG-NME1 293 cells which were metabolically labeled with Arg (¹³C₆/¹⁵N₄) and Lys (¹³C₆). These “heavy” labeled cells were used as an internal negative control for pHis mAb binding. Non-labeled, “light” FLAG-NME1 293 cells were cultured in parallel. For cell lysis, precautions were taken to preserve pHis and avoid salts and detergents that can interfere with downstream MS analysis. Both “heavy” and “light” cells were lysed under identical denaturing conditions to inhibit phosphatase activity and alkaline pH was used to stabilize pHis. All cell lysates (10×10 cm² plates) were prepared by rinsing cells (80-90% confluent) with cold TD buffer and scraping cells into 500 ul cold Denaturing Lysis Buffer (100 mM sodium carbonate pH 10 [60% Na₂CO₃/40% NaHCO₃], 6 M urea, 30 mM octyl-β-D-glucopyranoside supplemented with protease inhibitors (PMSF, pepstatin, leupeptin and aprotinin)). All “light” lysates were pooled together and all “heavy” lysates were pooled prior to sonication and clarification (10 min @ 15,000×g, 4° C.). Light lysates were set aside on ice and kept at pH 10 to preserve pHis while the heavy lysates were treated to reduce or abolish pHis by acidification (pH 6) and moderate heating (65° C. for 30 min). The combination of decreased pH and moderate heat treatment was found to be important for significant reduction of pHis, since neither treatment alone was sufficient. Extreme heating (e.g. 95-100° C.) was avoided to decrease carbamylation of proteins in the presence of urea. Both “heavy” and “light” lysates were then diluted 1:5 with Wash/Binding Buffer to decrease urea concentration to 1 M and neutralized pH to 8. The “heavy” and “light” lysates were pooled and passed over 1-pHis and 3-pHis mAb columns 2 times. The column was washed four times with 10 ml Wash/Binding Buffer. pHis proteins were eluted in three fractions (E1 to E3) with 6×600 ul 100 mM triethylamine (TEA), pH 11. Samples were saved from each fraction for analysis by SDS-PAGE and immunoblotting (FIG. 11). Elution fractions were frozen on dry ice and lyophilized overnight to remove volatile buffer components. Elution fractions were stored at −80° C. until LC-MS/MS was performed. A full list of proteins identified and the SILAC ratio for each peptide quantified using this method is shown in Example 13 and peptides from known pHis proteins quantified using this method are listed in Table 1.

The 1-pHis and 3-pHis mAb columns were tested for isomer selectivity by mixing pNME1 (1-pHis positive control) and pPGAM (3-pHis positive control) together. In vitro phosphorylation reactions using 80 μg recombinant NME1 or PGAM were performed separately in 800 μl TMD buffer with 1 mM ATP at RT or 2,3-DPG at 30° C. respectively for 10 min. 2 ml Sodium carbonate buffer pH 10 with 10 M urea (6 M final concentration) was then added to each reaction to denature proteins, stabilize pHis and replicate the alkaline and denaturing lysis conditions used to prepare cell lysates for the pHis mAb immunoaffinity purification described above. The two reactions were then pooled together (2.8 ml pNME1+2.8 ml pPGAM) and diluted 1:5 with 28 ml Wash/Binding Buffer to reduce urea to 1 M and pH to 8. 1-pHis and 3-pHis mAb columns were equilibrated with Wash/Binding Buffer and the half of the diluted pNME1 and pPGAM reaction mixtures were passed over each pHis mAb column two times. The columns were washed with 4×10 ml Wash/Binding Buffer and 3×600 μl elution fractions were collected using 100 mM TEA pH 11. Samples of each elution were mixed with 5×pH 8.8 sample buffer and stored at −80° C. for analysis by SDS-PAGE and immunoblotting with pHis, NME1 and PGAM antibodies.

Mass Spectrometry:

Lyophilized elution samples were first denatured in 8 M urea and then reduced and alkylated with 10 mM TCEP and 55 mM iodoacetamide respectively. The samples were diluted to 2 M urea with 100 mM Tris pH 8.5 and then digested with trypsin [Promega] overnight at 37° C. Each protein digest was pressure-loaded onto 250 micron i.d. fused silica capillary [Polymicro Technologies] columns with a Kasil frit packed with 3 cm of 5 micron C18 resin [Phenomenex]. After desalting, each column was connected to a 100 micron i.d. fused silica capillary [Polymicro Technologies] analytical column with a 5 micron pulled-tip, packed with 10 cm of 5 micron C18 resin [Phenomenex]. Each column was placed inline with a 1200 quaternary HPLC pump [Agilent] and the eluted peptides were electrosprayed directly into a LTQ Orbitrap Velos mass spectrometer [Thermo Scientific]. The buffer solutions used were 5% acetonitrile/0.1% formic acid (buffer A) and 80% acetonitrile/0.1% formic acid (buffer B). The 120 minute elution gradient had the following profile: 10% buffer B at 10 minutes, to 45% buffer B at 90 minutes, to 100% buffer B at 100 minutes and continuing to 110 minutes. A cycle consisted of one full scan mass spectrum (300-1600 m/z) at 60 K resolution followed by up to 20 data-dependent collision induced dissociation (CID) MS/MS spectra. Charge state exclusion was selected such that only +2 and +3 ions were selected for fragmentation. Dynamic exclusion was set at 120 seconds. Application of mass spectrometer scan functions and HPLC solvent gradients were controlled by the Xcalibur data system [Thermo Scientific].

MS/MS spectra were extracted using RawXtract (version 1.9.9) (McDonald et al., 2004). MS/MS spectra were searched with the ProLuCID algorithm (Eng et al., 1994) against a human Uniprot database concatenated to a decoy database in which the sequence for each entry in the original database was reversed (Peng et al., 2003). For protein identifications, the “light” ProLuCID search was performed using no enzyme specificity and static modification of cysteine due to carboxyamidomethylation (57.02146). A second “heavy” ProLuCID search was performed and additionally considered static modification of arginine (10.008269) and lysine (6.020129). The data was searched using a precursor mass tolerance of 50 ppm and a fragment ion mass tolerance of 600 ppm. Both “light” and “heavy” ProLuCID search results were assembled and filtered using the DTASelect (version 2.0) algorithm (Tabb et al., 2002). A minimum of one peptide was required for each protein identification and peptides were required to be fully tryptic. All peptide-spectra matches had less than 10 ppm mass error. The protein false positive rate was below one percent for all experiments. Quantification was performed using the Census algorithm (Park et al., 2008). The proline conversion correction option was selected. Peptide SILAC ratios were normalized based on the analysis of pre-column mixed lysates (data not shown). A determinant factor of 0.8 and a singleton profile score of 0.9 were used to filter quantified peptides. The composite score was selected for reporting SILAC ratios. For each quantified protein, three pieces of information are reported: the number of quantified peptides, the median peptide SILAC ratio and all peptide SILAC ratios. SILAC ratios reported are the untreated (“light”) sample divided by the acid/heat-treated (“heavy”) sample. A SILAC ratio greater than two was considered to be indicative of specific binding to the pHis columns.

Immunofluorescence:

Primary murine macrophages were differentiated from bone marrow progenitors (Zhang et al., 2008) plated on cover slips and incubated 0/N in fresh medium. Cells were incubated with 10 μg/ml Oregon Green-Dextran®488 and/or LysoTracker (50 nM) for 1-2 hr prior to fixation with 4% PFA for 10 min. Negative controls were performed by boiling slides for 5-10 min in 0.01 M citrate buffer or by pre-incubation of pHis mAbs with pTza blocking peptides [5 g/ml]. Cells were permeabilized in blocking buffer (PBS, 5% serum (2^{nd} Ab species), 2% BSA, 0.1% Tween) with 0.1% Triton-X100 for 1 hr at 4° C. Primary antibodies were diluted to 1 μg/ml in blocking buffer and incubated with slides for 2 hr at 4° C. Slides were washed 5× with cold PBS+0.1% Tween and incubated with 2^{nd} Ab diluted 1:400 in blocking buffer for 1 hr at 4° C. Slides were mounted on cover slips after washing 5× with cold PBS+0.1% Tween. See also Extended Experimental Procedures for immunostaining of HeLa cells.

1-pHis and 3-pHis Immunofluorescence Staining of HeLa Cells:

HeLa cells were plated on cover slips in 6-well plates and grown until 30-50% confluent. Cells were washed with sterile filtered PBS (pH 7.4) and fixed for 20 min at RT in 4% PFA (16% PFA diluted 1:4 in PBS). After fixation, cells were washed 2× with PBS and then permeabilized with PBS (pH 9.0)+0.1% Triton X-100 at RT for 15 min. Cells were then washed 3× with PBS (pH 9.0) before blocking in sterile filtered, 0.1% TBST with 4% BSA at RT for 30 min. Cover slips were transferred to parafilm, incubated with primary antibodies (1-pHis mAb SC1-1, diluted 1:100 [FIG. 10A]) at RT for 90 min and washed 3× with 0.1% TBST for 5 min. Secondary antibodies (anti-rabbit 488 nm and anti-mouse 568 nm) were diluted 1:2,000 in TBST plus 1% BSA and incubated with coverslips at RT for 60 min in the dark. Cells were then washed 3 times with TBST at RT for 5 min. To stain nuclei, coverslips were incubated with PBS plus DAPI at 1:4,000 for 2 min and washed 3 times with PBS. Alternative methods for fixation were used for co-staining experiments. Methanol fixation (Aurora A and α-tubulin) was performed by incubating cells at −20° C. for 15 min in methanol. Pre-permeabilization (γ-tubulin and α-tubulin) was performed by incubation of cells 45 sec in 0.5% Triton X-100 followed by 4% PFA for 15 min at pH 9.0. Coverslips were mounted and stored in the dark prior to scanning on a confocal microscope under 60× magnification.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. A method for detecting the presence of a tumor in a subject, comprising:
contacting a sample comprising polypeptides from the subject with a monoclonal antibody or antigen binding fragment thereof under conditions sufficient to form an immune complex with a polypeptide comprising a histidine phosphorylated at N3 (3-pHis) if present in the sample, wherein the monoclonal antibody comprises a heavy chain variable region and a light chain variable region, wherein the heavy chain variable region comprises a H-CDR1, a H-CDR2, and a H-CDR3, wherein the antibody or antigen binding fragment comprises:
a) the H-CDR1, the H-CDR2, and the H-CDR3 of the heavy chain variable region of the amino acid sequence set forth as SEQ ID NO: 1;
b) the H-CDR1, the H-CDR2, and the H-CDR3 of the heavy chain variable region of the amino acid sequence set forth as SEQ ID NO: 2;
c) the H-CDR1, the H-CDR2, and the H-CDR3 of the heavy chain variable region of the amino acid sequence set forth as SEQ ID NO: 3; or
d) the H-CDR1, the H-CDR2, and the H-CDR3 of the heavy chain variable region of the amino acid sequence set forth as SEQ ID NO: 4,
and wherein the monoclonal antibody specifically binds a polypeptide comprising a histidine phosphorylated at N3 (3-pHis);
detecting the presence of one or more polypeptides that are phosphorylated at N3; and
comparing the amount of the one or more polypeptides that are phosphorylated at N3 to a control,
wherein a change in the amount of the one or more polypeptides that are phosphorylated at N3 as compared to the control indicates the presence of the tumor in the subject.

2. The method of claim 1, wherein the light chain variable region comprises a L-CDR1, a L-CDR2, and a L-CDR3, wherein the antibody or antigen binding fragment comprises:
a) the L-CDR1, the L-CDR2, and the L-CDR3 of the light chain variable region of the amino acid sequence set forth as SEQ ID NO: 5;
b) the L-CDR1, the L-CDR2, and the L-CDR3 of the light chain variable region of the amino acid sequence set forth as SEQ ID NO: 6;
c) the L-CDR1, the L-CDR2, and the L-CDR3 of the light chain variable region of the amino acid sequence set forth as SEQ ID NO: 7; or
d) the L-CDR1, the L-CDR2, and the L-CDR3 of the light chain variable region of the amino acid sequence set forth as SEQ ID NO: 8.

3. The method of claim 1, wherein
a) the HCDR1, HCDR2, and HCDR3 of the monoclonal antibody comprise amino acids 28, 45-52, and 88-97 of SEQ ID NO: 1, respectively, and the LCDR1, LCDR2, and LCDR3 of the monoclonal antibody comprise amino acids 28-22, 51-53, and 90-102 of SEQ ID NO: 5 respectively;
b) the HCDR1, HCDR2, and HCDR3 of the monoclonal antibody comprise amino acids 21-28, 46-52, and 91-101 of SEQ ID NO: 2, respectively, and the LCDR1, LCDR2, and LCDR3 of the monoclonal antibody comprise amino acids 27-34, 52-54, 91-103 of SEQ ID NO: 6, respectively;
c) the HCDR1, HCDR2, and HCDR3 of the monoclonal antibody comprise amino acids 24-31, 49-55, 94-104 of SEQ ID NO: 3, respectively, and the LCDR1, LCDR2, and LCDR3 of the monoclonal antibody comprise amino acids 27-34, 52-54, and 91-109 of SEQ ID NO: 7, respectively; or
d) the HCDR1, HCDR2, and HCDR3 of the monoclonal antibody comprise amino acids 24-31, 49-55, 94-104 of SEQ ID NO: 4, respectively, and the LCDR1, LCDR2, and LCDR3 of the monoclonal antibody comprise amino acids amino acids 27-33, 51-53 and 90-102 of SEQ ID NO: 8, respectively.

4. The method of claim 3, wherein
a) the heavy chain variable region of the monoclonal antibody comprises amino acids 1-108 of SEQ ID NO: 1 and the light chain variable region of the monoclonal antibody comprises amino acids 1-113 of SEQ ID NO: 5;
b) the heavy chain variable region of the monoclonal antibody comprises amino acids 1-112 of SEQ ID NO: 2 and the light chain variable region of the monoclonal antibody comprises amino acids 1-114 of SEQ ID NO: 6;
c) the heavy chain variable region of the monoclonal antibody comprises amino acid 1-115 of SEQ ID NO: 3 and the light chain variable region of the monoclonal antibody comprises amino acids 1-120 of SEQ ID NO: 7, or
d) the heavy chain variable region of the monoclonal antibody comprises amino acids 1-115 of SEQ ID NO: 4 and the light chain variable region of the monoclonal antibody comprises amino acids 1-113 of SEQ ID NO: 8.

5. The method of claim 1, wherein the control represents an amount of the one or more polypeptides that are phosphorylated at N3 in a sample from a healthy subject or a standard value.

6. A method of determining if a subject with a tumor is responsive to a chemotherapeutic agent, comprising
contacting a tumor sample comprising polypeptides from the subject with a monoclonal antibody or antigen binding fragment thereof under conditions sufficient to form an immune complex with a polypeptide comprising a histidine phosphorylated at N3 (3-pHis) if present in the sample, wherein the monoclonal antibody comprises a heavy chain variable region and a light chain variable region, wherein the heavy chain variable region comprises a H-CDR1, a H-CDR2, and a H-CDR3, wherein the antibody or antigen binding fragment comprises:
a) the H-CDR1, the H-CDR2, and the H-CDR3 of the heavy chain variable region of the amino acid sequence set forth as SEQ ID NO: 1;
b) the H-CDR1, the H-CDR2, and the H-CDR3 of the heavy chain variable region of the amino acid sequence set forth as SEQ ID NO: 2;
c) the H-CDR1, the H-CDR2, and the H-CDR3 of the heavy chain variable region of the amino acid sequence set forth as SEQ ID NO: 3; or
d) the H-CDR1, the H-CDR2, and the H-CDR3 of the heavy chain variable region of the amino acid sequence set forth as SEQ ID NO: 4,
and wherein the monoclonal antibody specifically binds a polypeptide comprising a histidine phosphorylated at N3 (3-pHis);
detecting the presence of one or more polypeptides phosphorylated at N3; and
comparing the amount of the one or more polypeptides phosphorylated at N3 to a control,
wherein a change in the amount of the one or more polypeptides phosphorylated at N3 as compared to a control indicates that the chemotherapeutic agent is of use for treating the subject.

7. The method of claim 6, wherein the light chain variable region comprises a L-CDR1, a L-CDR2, and a L-CDR3, wherein the antibody or antigen binding fragment comprises:
a) the L-CDR1, the L-CDR2, and the L-CDR3 of the light chain variable region of the amino acid sequence set forth as SEQ ID NO: 5;
b) the L-CDR1, the L-CDR2, and the L-CDR3 of the light chain variable region of the amino acid sequence set forth as SEQ ID NO: 6;
c) the L-CDR1, the L-CDR2, and the L-CDR3 of the light chain variable region of the amino acid sequence set forth as SEQ ID NO: 7; or
d) the L-CDR1, the L-CDR2, and the L-CDR3 of the light chain variable region of the amino acid sequence set forth as SEQ ID NO: 8.

8. The method of claim 6, wherein
a) the HCDR1, HCDR2, and HCDR3 of the monoclonal antibody comprise amino acids 28, 45-52, and 88-97 of SEQ ID NO: 1, respectively, and the LCDR1, LCDR2, and LCDR3 of the monoclonal antibody comprise amino acids 28-22, 51-53, and 90-102 of SEQ ID NO: 5 respectively;
b) the HCDR1, HCDR2, and HCDR3 of the monoclonal antibody comprise amino acids 21-28, 46-52, and 91-101 of SEQ ID NO: 2, respectively, and the LCDR1, LCDR2, and LCDR3 of the monoclonal antibody comprise amino acids 27-34, 52-54, 91-103 of SEQ ID NO: 6, respectively;
c) the HCDR1, HCDR2, and HCDR3 of the monoclonal antibody comprise amino acids 24-31, 49-55, 94-104 of SEQ ID NO: 3, respectively, and the LCDR1, LCDR2, and LCDR3 of the monoclonal antibody comprise amino acids 27-34, 52-54, and 91-109 of SEQ ID NO: 7, respectively; or
d) the HCDR1, HCDR2, and HCDR3 of the monoclonal antibody comprise amino acids 24-31, 49-55, 94-104 of SEQ ID NO: 4, respectively, and the LCDR1, LCDR2, and LCDR3 of the monoclonal antibody comprise amino acids amino acids 27-33, 51-53 and 90-102 of SEQ ID NO: 8, respectively.

9. The method of claim 6, wherein
a) the heavy chain variable region of the monoclonal antibody comprises amino acids 1-108 of SEQ ID NO: 1 and the light chain variable region of the monoclonal antibody comprises amino acids 1-113 of SEQ ID NO: 5;
b) the heavy chain variable region of the monoclonal antibody comprises amino acids 1-112 of SEQ ID NO: 2 and the light chain variable region of the monoclonal antibody comprises amino acids 1-114 of SEQ ID NO: 6;
c) the heavy chain variable region of the monoclonal antibody comprises amino acid 1-115 of SEQ ID NO: 3 and the light chain variable region of the monoclonal antibody comprises amino acids 1-120 of SEQ ID NO: 7, or
d) the heavy chain variable region of the monoclonal antibody comprises amino acids 1-115 of SEQ ID NO: 4 and the light chain variable region of the monoclonal antibody comprises amino acids 1-113 of SEQ ID NO: 8.

10. The method of claim 6, wherein the control is an amount of the one or more polypeptides that are phosphorylated at N3 in a sample from the subject prior to treatment with the chemotherapeutic agent or a standard value.

11. A method of identifying an antibiotic, comprising
contacting a bacterial cell expressing a histidine kinase and a cognate response regulator with an agent of interest,
measuring an amount of phosphorylated histidine kinase and/or cognate response regulator bound by a monoclonal antibody or antigen binding fragment thereof under conditions sufficient to form an immune complex with a polypeptide comprising a histidine phosphorylated at N3 (3-pHis), wherein the monoclonal antibody comprises a heavy chain variable region and a light chain variable region, wherein the heavy chain variable region comprises a H-CDR1, a H-CDR2, and a H-CDR3, wherein the antibody or antigen binding fragment comprises:
a) the H-CDR1, the H-CDR2, and the H-CDR3 of the heavy chain variable region of the amino acid sequence set forth as SEQ ID NO: 1;
b) the H-CDR1, the H-CDR2, and the H-CDR3 of the heavy chain variable region of the amino acid sequence set forth as SEQ ID NO: 2;
c) the H-CDR1, the H-CDR2, and the H-CDR3 of the heavy chain variable region of the amino acid sequence set forth as SEQ ID NO: 3; or
d) the H-CDR1, the H-CDR2, and the H-CDR3 of the heavy chain variable region of the amino acid sequence set forth as SEQ ID NO: 4, and wherein the monoclonal antibody specifically binds a polypeptide comprising a histidine phosphorylated at N3 (3-pHis); and
detecting the presence of histidine kinase and/or cognate response regulator phosphorylated at N3;
wherein a decrease in the amount of histidine kinase and a cognate response regulator phosphorylated at N3 as compared to a control indicates the agent of interest is an antibiotic.

12. The method of claim 11, wherein the light chain variable region comprises a L-CDR1, a L-CDR2, and a L-CDR3, wherein the antibody or antigen binding fragment comprises:
a) the L-CDR1, the L-CDR2, and the L-CDR3 of the light chain variable region of the amino acid sequence set forth as SEQ ID NO: 5;
b) the L-CDR1, the L-CDR2, and the L-CDR3 of the light chain variable region of the amino acid sequence set forth as SEQ ID NO: 6;
c) the L-CDR1, the L-CDR2, and the L-CDR3 of the light chain variable region of the amino acid sequence set forth as SEQ ID NO: 7; or
d) the L-CDR1, the L-CDR2, and the L-CDR3 of the light chain variable region of the amino acid sequence set forth as SEQ ID NO: 8.

13. The method of claim 11, wherein
a) the HCDR1, HCDR2, and HCDR3 of the monoclonal antibody comprise amino acids 28, 45-52, and 88-97 of SEQ ID NO: 1, respectively, and the LCDR1, LCDR2, and LCDR3 of the monoclonal antibody comprise amino acids 28-22, 51-53, and 90-102 of SEQ ID NO: 5 respectively;
b) the HCDR1, HCDR2, and HCDR3 of the monoclonal antibody comprise amino acids 21-28, 46-52, and 91-101 of SEQ ID NO: 2, respectively, and the LCDR1, LCDR2, and LCDR3 of the monoclonal antibody comprise amino acids 27-34, 52-54, 91-103 of SEQ ID NO: 6, respectively;
c) the HCDR1, HCDR2, and HCDR3 of the monoclonal antibody comprise amino acids 24-31, 49-55, 94-104 of SEQ ID NO: 3, respectively, and the LCDR1, LCDR2, and LCDR3 of the monoclonal antibody comprise amino acids 27-34, 52-54, and 91-109 of SEQ ID NO: 7, respectively; or
d) the HCDR1, HCDR2, and HCDR3 of the monoclonal antibody comprise amino acids 24-31, 49-55, 94-104 of SEQ ID NO: 4, respectively, and the LCDR1, LCDR2, and LCDR3 of the monoclonal antibody comprise amino acids amino acids 27-33, 51-53 and 90-102 of SEQ ID NO: 8, respectively.

14. The method of claim 11, wherein
a) the heavy chain variable region of the monoclonal antibody comprises amino acids 1-108 of SEQ ID NO: 1 and the light chain variable region of the monoclonal antibody comprises amino acids 1-113 of SEQ ID NO: 5;
b) the heavy chain variable region of the monoclonal antibody comprises amino acids 1-112 of SEQ ID NO: 2 and the light chain variable region of the monoclonal antibody comprises amino acids 1-114 of SEQ ID NO: 6;
c) the heavy chain variable region of the monoclonal antibody comprises amino acid 1-115 of SEQ ID NO: 3 and the light chain variable region of the monoclonal antibody comprises amino acids 1-120 of SEQ ID NO: 7, or
d) the heavy chain variable region of the monoclonal antibody comprises amino acids 1-115 of SEQ ID NO: 4 and the light chain variable region of the monoclonal antibody comprises amino acids 1-113 of SEQ ID NO: 8.

15. The method of claim 11, wherein the control is an amount of histidine kinase and a cognate response regulator in a cell not contacted with the agent of interest or a standard value.

16. The method of claim 11, wherein the bacterial cell is a gram negative or gram positive bacterial cell.

17. A method of identifying specific polypeptides that comprise a histidine phosphorylated at N3 in a sample from a subject, comprising
contacting a sample comprising polypeptides from the subject with a solid substrate comprising a monoclonal antibody or antigen binding fragment thereof under conditions sufficient to form an immune complex with a polypeptide comprising a histidine phosphorylated at N3 (3-pHis), wherein the monoclonal antibody comprises a heavy chain variable region and a light chain variable region, wherein the heavy chain variable region comprises a H-CDR1, a H-CDR2, and a H-CDR3, wherein the antibody or antigen binding fragment comprises:
a) the H-CDR1, the H-CDR2, and the H-CDR3 of the heavy chain variable region of the amino acid sequence set forth as SEQ ID NO: 1;
b) the H-CDR1, the H-CDR2, and the H-CDR3 of the heavy chain variable region of the amino acid sequence set forth as SEQ ID NO: 2;
c) the H-CDR1, the H-CDR2, and the H-CDR3 of the heavy chain variable region of the amino acid sequence set forth as SEQ ID NO: 3; or
d) the H-CDR1, the H-CDR2, and the H-CDR3 of the heavy chain variable region of the amino acid sequence set forth as SEQ ID NO: 4,
and wherein the monoclonal antibody specifically binds a polypeptide comprising a histidine phosphorylated at N3 (3-pHis);
eluting the polypeptides from the solid substrate; and
performing mass spectrometry or an immunoassay to detect the presence of one or more specific proteins;
thereby identifying specific polypeptides including a histidine phosphorylated at N3 in the sample.

18. The method of claim 17, wherein the light chain variable region comprises a L-CDR1, a L-CDR2, and a L-CDR3, wherein the antibody or antigen binding fragment comprises:
a) the L-CDR1, the L-CDR2, and the L-CDR3 of the light chain variable region of the amino acid sequence set forth as SEQ ID NO: 5;
b) the L-CDR1, the L-CDR2, and the L-CDR3 of the light chain variable region of the amino acid sequence set forth as SEQ ID NO: 6;
c) the L-CDR1, the L-CDR2, and the L-CDR3 of the light chain variable region of the amino acid sequence set forth as SEQ ID NO: 7; or
d) the L-CDR1, the L-CDR2, and the L-CDR3 of the light chain variable region of the amino acid sequence set forth as SEQ ID NO: 8.

19. The method of claim 17, wherein
a) the HCDR1, HCDR2, and HCDR3 of the monoclonal antibody comprise amino acids 28, 45-52, and 88-97 of SEQ ID NO: 1, respectively, and the LCDR1, LCDR2, and LCDR3 of the monoclonal antibody comprise amino acids 28-22, 51-53, and 90-102 of SEQ ID NO: 5 respectively;
b) the HCDR1, HCDR2, and HCDR3 of the monoclonal antibody comprise amino acids 21-28, 46-52, and 91-101 of SEQ ID NO: 2, respectively, and the LCDR1, LCDR2, and LCDR3 of the monoclonal antibody comprise amino acids 27-34, 52-54, 91-103 of SEQ ID NO: 6, respectively;
c) the HCDR1, HCDR2, and HCDR3 of the monoclonal antibody comprise amino acids 24-31, 49-55, 94-104 of SEQ ID NO: 3, respectively, and the LCDR1, LCDR2, and LCDR3 of the monoclonal antibody comprise amino acids 27-34, 52-54, and 91-109 of SEQ ID NO: 7, respectively; or
d) the HCDR1, HCDR2, and HCDR3 of the monoclonal antibody comprise amino acids 24-31, 49-55, 94-104 of SEQ ID NO: 4, respectively, and the LCDR1, LCDR2, and LCDR3 of the monoclonal antibody comprise amino acids amino acids 27-33, 51-53 and 90-102 of SEQ ID NO: 8, respectively.

20. The method of claim 17, wherein
a) the heavy chain variable region of the monoclonal antibody comprises amino acids 1-108 of SEQ ID NO: 1 and the light chain variable region of the monoclonal antibody comprises amino acids 1-113 of SEQ ID NO: 5;
b) the heavy chain variable region of the monoclonal antibody comprises amino acids 1-112 of SEQ ID NO: 2 and the light chain variable region of the monoclonal antibody comprises amino acids 1-114 of SEQ ID NO: 6;
c) the heavy chain variable region of the monoclonal antibody comprises amino acid 1-115 of SEQ ID NO: 3 and the light chain variable region of the monoclonal antibody comprises amino acids 1-120 of SEQ ID NO: 7, or
d) the heavy chain variable region of the monoclonal antibody comprises amino acids 1-115 of SEQ ID NO: 4 and the light chain variable region of the monoclonal antibody comprises amino acids 1-113 of SEQ ID NO: 8.

21. The method of claim 17, wherein the sample is from a subject with a tumor.

22. The method of claim 21, wherein the antigen binding fragment is a Fv, Fab, F(ab′)₂, scFV or a scFV₂ fragment.

23. The method of claim 21, wherein the monoclonal antibody or antigen binding fragment is conjugated to a detectable label.

24. The method of claim 23, wherein the detectable marker is a fluorescent, enzymatic, heavy metal or radioactive marker.
